(19) United States
(12) Patent Application Publication
(10) Pub. No.: US 2024/0043491 A1
(43) Pub. Date: Feb. 8, 2024

(54) IL-2 MUTEINS FOR TREATING AUTOIMMUNE AND INFLAMMATORY DISEASES

(71) Applicant: MERCK SHARP & DOHME LLC, Rahway, NJ (US)

(72) Inventors: Stephen E. ALVES, Cambridge, MA (US); Glareh AZADI, South San Francisco, CA (US); Maribel BEAUMONT, San Francisco, CA (US); Lia BENSO, Cambridge, MA (US); Alan C. CHENG, San Francisco, CA (US); Peter GEORGIEV, Chestnut Hill, MA (US); Brian E. HALL, Wellesley Hill, MA (US); Veronica M. JUAN, San Francisco, CA (US); Renee MOORE, Boston, MA (US); Venkataraman SRIRAMAN, Boston, MA (US); Jie ZHANG-HOOVER, Boston, MA (US)

(73) Assignee: MERCK SHARP & DOHME LLC, Rahway, NJ (US)

(21) Appl. No.: 18/258,397

(22) PCT Filed: Dec. 21, 2021
(86) PCT No.: PCT/US21/64476

§ 371 Date: Jun. 20, 2023

Related U.S. Application Data
(60) Provisional application No. 63/129,712, filed on Dec. 23, 2020.

(51) Int. Cl. C07K 14/55

(52) U.S. Cl. CPC C07K 14/55; A61K 38/00

(57) ABSTRACT

Provided herein are IL-2 muteins that bind to IL-2 receptor subunit but do not have measurable binding to IL-2 receptor subunit. Also provided are compositions, kits, methods, and uses involving such IL-2 muteins.

### CROSS-REFERENCE TO RELATED APPLICATIONS

This application claims the benefit of U.S. Provisional Application No. 63/129,712, filed Dec. 23, 2020, the content of which is hereby incorporated by reference in its entirety.

### FIELD

The present invention relates to IL-2 muteins that bind to the IL-2 receptor a subunit but do not have measurable binding to the IL-2 receptor β subunit. The invention also relates to compositions, kits, methods, and uses involving such IL-2 muteins.

### REFERENCE TO SEQUENCE LISTING SUBMITTED ELECTRONICALLY

The sequence listing of the present application is submitted electronically via EFS-Web as an ASCII formatted sequence listing with a file name “25184WOPCT-SEQLIST-21NOV.2021.TXT”, creation date of Nov. 21, 2021, and a size of 132 KB. This sequence listing submitted via EFS-Web is part of the specification and is herein incorporated by reference in its entirety.

### BACKGROUND

The biological activity of interleukin-2 (IL-2) is mediated through a multi-subunit IL-2 receptor (IL-2R) complex comprising two or three polypeptide subunits: CD25 (IL-2R α subunit) enhances affinity of the IL-2R complex to IL-2, while CD122 (IL-2R β subunit) and CD132 (IL-2R γ subunit) are required for signal transduction. The dimeric IL-2R β/γ complex and trimeric IL-2R α/β/γ complex are differentially expressed by various immune cell subtypes. For example, the high affinity trimeric IL-2R α/β/γ complex is constitutively expressed at high levels by regulatory T (Treg) cells and transiently expressed at lower levels by CD4+T effector cells, whereas the moderate affinity dimeric IL-2R β/γ complex is expressed primarily on CD8+ T cells and natural killer cells (Stauber et al., (2006) PNAS USA 103(8):2788-93; Malek and Castro, (2010) Immunity 33(2):153-165; Ross and Cantrell, (2018) Annu Rev Immunol 36:418-433). Thus, IL-2 muteins with a bias to bind to the dimeric or trimeric IL-2R complex can activate different cell types and mediate either immune activation or suppression, respectively. There remains unmet need to identify innovative IL-2 muteins that can more selectively activate IL-2R signals in distinct cell types to treat immune-mediated diseases, including biologically optimized IL-2 muteins selectively binding to the IL-2R α subunit for the treatment of autoimmune and inflammatory diseases.

### SUMMARY OF THE INVENTION

The present disclosure provides IL-2 muteins that bind to IL-2 receptor a subunit but do not have measurable binding to IL-2 receptor β subunit. Also provided herein are methods or uses involving such IL-2 muteins, compositions or kits comprising such IL-2 muteins, isolated nucleic acids and vectors comprising polynucleotide sequences encoding such IL-2 muteins, cells (e.g., host cells) comprising such isolated nucleic acids or vectors, and methods of producing such IL-2 muteins.

In one aspect, provided herein is an IL-2 mutein that binds to IL-2 receptor a subunit but does not bind directly to IL-2 receptor β subunit.

In certain embodiments, provided is an IL-2 mutein that comprises a first polypeptide comprising the amino acid sequence as set forth in SEQ ID NO:1 or 2 in which the D at position 19 of SEQ ID NO:1 or 2 is substituted with an N, and the P at position 33 of SEQ ID NO:1 or 2 is substituted with an R, wherein the polypeptide optionally comprises one or more additional amino acid substitutions relative to SEQ ID NO:1 or 2. In one embodiment, the IL-2 mutein comprises a first polypeptide comprising an amino acid sequence as set forth in SEQ ID NO:1 in which the D at position 19 of SEQ ID NO:1 is substituted with an N, and the P at position 33 of SEQ ID NO:1 is substituted with an R, wherein the polypeptide optionally comprises one or more additional amino acid substitutions relative to SEQ ID NO:1. In another embodiment, the IL-2 mutein comprises a first polypeptide comprising an amino acid sequence as set forth in SEQ ID NO:2 in which the D at position 19 of SEQ ID NO:2 is substituted with an N, and the P at position 33 of SEQ ID NO:2 is substituted with an R, wherein the polypeptide optionally comprises one or more additional amino acid substitutions relative to SEQ ID NO:2.

In specific embodiments of the IL-2 muteins described above, the one or more additional amino acid substitutions relative to SEQ ID NO:1 or SEQ ID NO:2 are selected from the group consisting of: an E to S substitution at position 67, a V to A substitution at position 68, an N to R substitution at position 70, and a Q to P substitution at position 73 of SEQ ID NO:1 or 2.

In some embodiments, provided is an IL-2 mutein that comprises a first polypeptide comprising the amino acid sequence as set forth in SEQ ID NO:1 or 2 in which the D at position 19 of SEQ ID NO:1 or 2 is substituted with an N, and the E at position 67 of SEQ ID NO:1 or 2 is substituted with an S, wherein the polypeptide optionally comprises one or more additional amino acid substitutions relative to SEQ ID NO:1 or 2. In one embodiment, the IL-2 mutein comprises a first polypeptide comprising an amino acid sequence as set forth in SEQ ID NO:1 in which the D at position 19 of SEQ ID NO:1 is substituted with an N and the E at position 67 of SEQ ID NO:1 is substituted with an S, wherein the polypeptide optionally comprises one or more additional amino acid substitutions relative to SEQ ID NO:1. In another embodiment, the IL-2 mutein comprises a first polypeptide comprising an amino acid sequence as set forth in SEQ ID NO:2 in which the D at position 19 of SEQ ID NO:2 is substituted with an N and the E at position 67 of SEQ ID NO:2 is substituted with an S, wherein the polypeptide optionally comprises one or more additional amino acid substitutions relative to SEQ ID NO:2.

In other embodiments, provided is an IL-2 mutein that comprises a first polypeptide comprising an amino acid sequence as set forth in SEQ ID NO:1 or 2 in which the D at position 19 of SEQ ID NO:1 or 2 is substituted with an N, the P at position 33 of SEQ ID NO:1 or 2 is substituted with an R, and the E at position 67 of SEQ ID NO:1 or 2 is substituted with an S, wherein the polypeptide optionally comprises one or more additional amino acid substitutions relative to SEQ ID NO:1 or 2. In one embodiment, the IL-2 mutein comprises a first polypeptide comprising an amino acid sequence as set forth in SEQ ID NO:1 in which the D at position 19 of SEQ ID NO:1 is substituted with an N, the P at position 33 of SEQ ID NO:1 is substituted with an R, and the E at position 67 of SEQ ID NO:1 is substituted with an S, wherein the polypeptide optionally comprises one or more additional amino acid substitutions relative to SEQ ID NO:1. 1. In another embodiment, the IL-2 mutein comprises a first polypeptide comprising an amino acid sequence as set forth in SEQ ID NO:2 in which the D at position 19 of SEQ ID NO:2 is substituted with an N, the P at position 33 of SEQ ID NO:2 is substituted with an R, and the E at position 67 of SEQ ID NO:2 is substituted with an S, wherein the polypeptide optionally comprises one or more additional amino acid substitutions relative to SEQ ID NO:2.

In yet other embodiments of the IL-2 muteins, the first polypeptide further comprises a V to A substitution at position 68, an N to R substitution at position 70, or a Q to P substitution at position 73 of SEQ ID NO:1 or 2.

In still other embodiments of the IL-2 muteins, the first polypeptide further comprises any two of the three following substitutions: a V to A substitution at position 68, an N to R substitution at position 70, or a Q to P substitution at position 73 of SEQ ID NO:1 or 2.

In yet still other embodiments of the IL-2 muteins, the first polypeptide further comprises a V to A substitution at position 68, an N to R substitution at position 70, and a Q to P substitution at position 73 of SEQ ID NO:1 or 2.

In certain embodiments, the IL-2 mutein comprises a first polypeptide comprising an amino acid sequence as set forth in SEQ ID NO:3, 4, 5, 6, 7, 8, 9, 10, 11, or 12. In one embodiment, the IL-2 mutein comprises a first polypeptide comprising the amino acid sequence as set forth in SEQ ID NO:3. In another embodiment, the IL-2 mutein comprises a first polypeptide comprising the amino acid sequence as set forth in SEQ ID NO:4. In yet another embodiment, the IL-2 mutein comprises a first polypeptide comprising the amino acid sequence as set forth in SEQ ID NO:5. In still another embodiment, the IL-2 mutein comprises a first polypeptide comprising the amino acid sequence as set forth in SEQ ID NO:6. In yet still another embodiment, the IL-2 mutein comprises a first polypeptide comprising the amino acid sequence as set forth in SEQ ID NO:7. In one embodiment, the IL-2 mutein comprises a first polypeptide comprising the amino acid sequence as set forth in SEQ ID NO:8. In another embodiment, the IL-2 mutein comprises a first polypeptide comprising the amino acid sequence as set forth in SEQ ID NO:9. In yet another embodiment, the IL-2 mutein comprises a first polypeptide comprising the amino acid sequence as set forth in SEQ ID NO:10. In still another embodiment, the IL-2 mutein comprises a first polypeptide comprising the amino acid sequence as set forth in SEQ ID NO:11. In yet still another embodiment, the IL-2 mutein comprises a first polypeptide comprising the amino acid sequence as set forth in SEQ ID NO: 12.

In some embodiments of the IL-2 muteins, the first polypeptide further comprises an amino acid sequence as set forth in SEQ ID NO:13, 14, or 15. In one embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO: 13. In another embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO: 14. In yet another embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO:15.

In other embodiments of the IL-2 muteins, the first polypeptide further comprises a linker as set forth in SEQ ID NO: 16. In one embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO: 13 and a linker as set forth in SEQ ID NO: 16. In another embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO: 14 and a linker as set forth in SEQ ID NO:16. In yet another embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO:15 and a linker as set forth in SEQ ID NO:16.

In various embodiments, the IL-2 mutein further comprises a second polypeptide. In some embodiments the first and second polypeptide are the same. In other embodiments, the first and second polypeptide are not the same.

In one embodiment, the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO: 14 and a second polypeptide comprising the amino acid sequence as set forth in SEQ ID NO: 17.

In one embodiment, the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO: 14 and a second polypeptide comprising the amino acid sequence as set forth in SEQ ID NO:55.

In another embodiment, the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO: 15 and a second polypeptide comprising the amino acid sequence as set forth in SEQ ID NO: 18.

In some embodiments, the IL-2 mutein comprises a first polypeptide and a second polypeptide, wherein the first polypeptide comprises an amino acid sequence as set forth in SEQ ID NO:19, 20, 21, 22, 23, 24, 25, 26, 27, or 28; and the second polypeptide comprises the amino acid sequence as set forth in SEQ ID NO:17. In one embodiment, the first polypeptide comprises the amino acid sequence as set forth in SEQ ID NO:19; and the second polypeptide comprises the amino acid sequence as set forth in SEQ ID NO: 17. In another embodiment, the first polypeptide comprises the amino acid sequence as set forth in SEQ ID NO:20; and the second polypeptide comprises the amino acid sequence as set forth in SEQ ID NO:17. In yet another embodiment, the first polypeptide comprises the amino acid sequence as set forth in SEQ ID NO:21; and the second polypeptide comprises the amino acid sequence as set forth in SEQ ID NO:17. In still another embodiment, the first polypeptide comprises the amino acid sequence as set forth in SEQ ID NO:22; and the second polypeptide comprises the amino acid sequence as set forth in SEQ ID NO:17. In yet still another embodiment, the first polypeptide comprises the amino acid sequence as set forth in SEQ ID NO:23; and the second polypeptide comprises the amino acid sequence as set forth in SEQ ID NO:17. In one embodiment, the first polypeptide comprises the amino acid sequence as set forth in SEQ ID NO:24; and the second polypeptide comprises the amino acid sequence as set forth in SEQ ID NO:17. In another embodiment, the first polypeptide comprises the amino acid sequence as set forth in SEQ ID NO:25; and the second polypeptide comprises the amino acid sequence as set forth in SEQ ID NO:17. In yet another embodiment, the first polypeptide comprises the amino acid sequence as set forth in SEQ ID NO:26; and the second polypeptide comprises an amino acid sequence as set forth in SEQ ID NO:17. In still another embodiment, the first polypeptide comprises the amino acid sequence as set forth in SEQ ID NO:27; and the second polypeptide comprises the amino acid sequence as set forth in SEQ ID NO:17. In yet still another embodiment, the first polypeptide comprises an amino acid sequence as set forth in SEQ ID NO:28; and the second polypeptide comprises an amino acid sequence as set forth in SEQ ID NO: 17.

In some embodiments, the IL-2 mutein comprises a first polypeptide and a second polypeptide, wherein the first polypeptide comprises an amino acid sequence as set forth in SEQ ID NO: 19, 20, 21, 22, 23, 24, 25, 26, 27, or 28; and the second polypeptide comprises the amino acid sequence as set forth in SEQ ID NO:55. In one embodiment, the first polypeptide comprises the amino acid sequence as set forth in SEQ ID NO:19; and the second polypeptide comprises the amino acid sequence as set forth in SEQ ID NO:55. In another embodiment, the first polypeptide comprises the amino acid sequence as set forth in SEQ ID NO:20; and the second polypeptide comprises the amino acid sequence as set forth in SEQ ID NO:55. In yet another embodiment, the first polypeptide comprises the amino acid sequence as set forth in SEQ ID NO:21; and the second polypeptide comprises the amino acid sequence as set forth in SEQ ID NO:55. In still another embodiment, the first polypeptide comprises the amino acid sequence as set forth in SEQ ID NO:22; and the second polypeptide comprises the amino acid sequence as set forth in SEQ ID NO:55. In yet still another embodiment, the first polypeptide comprises the amino acid sequence as set forth in SEQ ID NO:23; and the second polypeptide comprises the amino acid sequence as set forth in SEQ ID NO:55. In one embodiment, the first polypeptide comprises the amino acid sequence as set forth in SEQ ID NO:24; and the second polypeptide comprises the amino acid sequence as set forth in SEQ ID NO:55. In another embodiment, the first polypeptide comprises the amino acid sequence as set forth in SEQ ID NO:25; and the second polypeptide comprises the amino acid sequence as set forth in SEQ ID NO:55. In yet another embodiment, the first polypeptide comprises the amino acid sequence as set forth in SEQ ID NO:26; and the second polypeptide comprises an amino acid sequence as set forth in SEQ ID NO:55. In still another embodiment, the first polypeptide comprises the amino acid sequence as set forth in SEQ ID NO:27; and the second polypeptide comprises the amino acid sequence as set forth in SEQ ID NO:55. In yet still another embodiment, the first polypeptide comprises an amino acid sequence as set forth in SEQ ID NO:28; and the second polypeptide comprises an amino acid sequence as set forth in SEQ ID NO:55.

In certain embodiments, the IL-2 mutein comprises a first polypeptide and a second polypeptide, wherein the first polypeptide comprises an amino acid sequence as set forth in SEQ ID NO:29, 30, 31, 32, 33, 34, 35, 36, 37, or 38; and the second polypeptide comprises the amino acid sequence as set forth in SEQ ID NO:18. In one embodiment, the first polypeptide comprises the amino acid sequence as set forth in SEQ ID NO:29; and the second polypeptide comprises the amino acid sequence as set forth in SEQ ID NO:18. In another embodiment, the first polypeptide comprises the amino acid sequence as set forth in SEQ ID NO:30; and the second polypeptide comprises the amino acid sequence as set forth in SEQ ID NO:18. In yet another embodiment, the first polypeptide comprises the amino acid sequence as set forth in SEQ ID NO:31; and the second polypeptide comprises the amino acid sequence as set forth in SEQ ID NO: 18. In still another embodiment, the first polypeptide comprises an amino acid sequence as set forth in SEQ ID NO:32; and the second polypeptide comprises the amino acid sequence as set forth in SEQ ID NO:18. In yet still another embodiment, the first polypeptide comprises the amino acid sequence as set forth in SEQ ID NO:33; and the second polypeptide comprises the amino acid sequence as set forth in SEQ ID NO: 18. In one embodiment, the first polypeptide comprises the amino acid sequence as set forth in SEQ ID NO:34; and the second polypeptide comprises the amino acid sequence as set forth in SEQ ID NO:18. In another embodiment, the first polypeptide comprises the amino acid sequence as set forth in SEQ ID NO:35; and the second polypeptide comprises an amino acid sequence as set forth in SEQ ID NO: 18. In yet another embodiment, the first polypeptide comprises the amino acid sequence as set forth in SEQ ID NO:36; and the second polypeptide comprises the amino acid sequence as set forth in SEQ ID NO:18. In still another embodiment, the first polypeptide comprises the amino acid sequence as set forth in SEQ ID NO:37; and the second polypeptide comprises the amino acid sequence as set forth in SEQ ID NO:18. In yet still another embodiment, the first polypeptide comprises the amino acid sequence as set forth in SEQ ID NO:38; and the second polypeptide comprises the amino acid sequence as set forth in SEQ ID NO:18.

In certain embodiments, the IL-2 mutein comprises a first polypeptide and a second polypeptide, wherein the first and second polypeptides comprise an amino acid sequence that is the same. In certain embodiments, the amino acid sequence of the first polypeptide and the second polypeptide each comprises an amino acid sequence as set forth in SEQ ID NO: 45, 46, 47, 48, 49, 50, 51, 52, 53, or 54. In one embodiment, each of the first and second polypeptides comprises the amino acid sequence as set forth in SEQ ID NO: 45. In one embodiment, each of the first and second polypeptides comprises the amino acid sequence as set forth in SEQ ID NO: 46. In one embodiment, each of the first and second polypeptides comprises the amino acid sequence as set forth in SEQ ID NO: 47. In one embodiment, each of the first and second polypeptides comprises the amino acid sequence as set forth in SEQ ID NO: 48. In one embodiment, each of the first and second polypeptides comprises the amino acid sequence as set forth in SEQ ID NO: 49. In one embodiment, each of the first and second polypeptides comprises the amino acid sequence as set forth in SEQ ID NO: 50. In one embodiment, each of the first and second polypeptides comprises the amino acid sequence as set forth in SEQ ID NO: 51. In one embodiment, each of the first and second polypeptides comprises the amino acid sequence as set forth in SEQ ID NO: 52. In one embodiment, each of the first and second polypeptides comprises the amino acid sequence as set forth in SEQ ID NO: 53. In one embodiment, each of the first and second polypeptide comprises the amino acid sequence as set forth in SEQ ID NO: 54.

In another aspect, provided is a pharmaceutical composition comprising any one of the various IL-2 muteins described herein and a pharmaceutically acceptable carrier.

In yet another aspect, provided is a method of treating an IL-2-mediated disease in a subject, comprising administering to the subject a therapeutically effective amount of any one of the various IL-2 muteins described herein or any one of the various pharmaceutical compositions described herein.

In certain embodiments, the IL-2-mediated disease is an immune disease. In other embodiments, the IL-2-mediated disease is an autoimmune disease.

In some embodiments, the immune disease is rheumatoid arthritis, Crohn's disease, psoriasis, psoriatic arthritis, multiple sclerosis, systemic lupus erythematosus (SLE), cutaneous lupus erythematosus (CLE), lupus nephritis, ankylosing spondylitis, type I diabetes, Sjogren's syndrome, ulcerative colitis, neuromyelitis optica, celiac disease, scleroderma, temporal arteritis, atopic dermatitis, alopecia areata, graft versus host disease (GVHD), autoimmune hepatitis, primary sclerosing cholangitis, or inflammatory myopathy. In one embodiment, the immune disease is rheumatoid arthritis. In another embodiment, the immune disease is Crohn's disease. In yet another embodiment, the immune disease is psoriasis. In still another embodiment, the immune disease is psoriatic arthritis. In yet still another embodiment, the immune disease is multiple sclerosis. In one embodiment, the immune disease is SLE. In another embodiment, the immune disease is CLE. In yet another embodiment, the immune disease is lupus nephritis. In still another embodiment, the immune disease is ankylosing spondylitis. In yet still another embodiment, the immune disease is type I diabetes. In one embodiment, the immune disease is Sjogren's syndrome. In another embodiment, the immune disease is ulcerative colitis. In yet another embodiment, the immune disease is neuromyelitis optica. In still another embodiment, the immune disease is celiac disease. In yet still another embodiment, the immune disease is scleroderma. In one embodiment, the immune disease is temporal arteritis. In another embodiment, the immune disease is atopic dermatitis. In yet another embodiment, the immune disease is alopecia areata. In still another embodiment, the immune disease is GVHD. In yet still another embodiment, the immune disease is autoimmune hepatitis. In one embodiment, the immune disease is primary sclerosing cholangitis. In another embodiment, the immune disease is inflammatory myopathy.

In still another aspect, provided is a method of selectively activating T regulatory cells without activating CD8+ T cells in a subject, comprising administering to the subject in need thereof a therapeutically effective amount of any one of the various IL-2 muteins described herein or any one of the various pharmaceutical composition described herein.

In yet still another aspect, provided is a method of selectively activating cells that express IL-2 receptor β subunit but not activating cells that express IL-2 receptor α subunit in a subject, comprising administering to the subject in need thereof a therapeutically effective amount of any one of the various IL-2 muteins described herein or any one of the various pharmaceutical composition described herein.

In one aspect, provided is an isolated nucleic acid comprising a sequence of nucleotides that encodes any one of the various polypeptides of the various IL-2 muteins described herein. In one embodiment, the isolated nucleic acid encodes any one of the various first polypeptides of the various IL-2 muteins described herein. In another embodiment, the isolated nucleic acid encodes any one of the various second polypeptides of the various IL-2 muteins described herein. In yet another embodiment, the isolated nucleic acid encodes any one of the various first polypeptides and the various second polypeptides of the various IL-2 muteins described herein.

In another aspect, provided is an expression vector comprising any one of the various isolated nucleic acids described herein.

In yet another aspect, provided is a host cell comprising any one of the various isolated nucleic acids described herein or any one of the various expression vectors described herein.

In still another aspect, provided is a method of producing any one of the various IL-2 muteins described herein. In one embodiment, the method comprises culturing any one of the various host cells described herein under conditions wherein the IL-2 mutein is expressed. In another embodiment, the method comprises expressing any one of the various expression vectors described herein under conditions wherein the IL-2 mutein is expressed. In yet another embodiment, the method comprises expressing any one of the various isolated nucleic acids described herein under conditions wherein the IL-2 mutein is expressed.

In yet still another aspect, provided is use of any one of the various IL-2 muteins described herein or any one of the various pharmaceutical compositions described herein to treat an IL-2-mediated disease in a subject.

In one aspect, provided is use of any one of the various IL-2 muteins described herein or any one of the various pharmaceutical compositions described herein for the treatment of an IL-2-mediated disease in a subject.

In another aspect, provided is use of any one of the various IL-2 muteins described herein or any one of the various pharmaceutical compositions described herein for the preparation of a medicament to treat an IL-2-mediated disease in a subject.

In some embodiments of the various uses described herein, the IL-2-mediated disease is an immune disease. In other embodiments of the various uses described herein, the IL-2-mediated disease is an autoimmune disease.

In certain embodiments of the various uses described herein, the immune disease is rheumatoid arthritis, Crohn's disease, psoriasis, psoriatic arthritis, multiple sclerosis, systemic lupus erythematosus (SLE), cutaneous lupus erythematosus (CLE), lupus nephritis, ankylosing spondylitis, type I diabetes, Sjogren's syndrome, ulcerative colitis, neuromyelitis optica, celiac disease, scleroderma, temporal arteritis, atopic dermatitis, alopecia areata, graft versus host disease (GVHD), autoimmune hepatitis, primary sclerosing cholangitis, or inflammatory myopathy. In one embodiment, the immune disease is rheumatoid arthritis. In another embodiment, the immune disease is Crohn's disease. In yet another embodiment, the immune disease is psoriasis. In still another embodiment, the immune disease is psoriatic arthritis. In yet still another embodiment, the immune disease is multiple sclerosis. In one embodiment, the immune disease is SLE. In another embodiment, the immune disease is CLE. In yet another embodiment, the immune disease is lupus nephritis. In still another embodiment, the immune disease is ankylosing spondylitis. In yet still another embodiment, the immune disease is type I diabetes. In one embodiment, the immune disease is Sjogren's syndrome. In another embodiment, the immune disease is ulcerative colitis. In yet another embodiment, the immune disease is neuromyelitis optica. In still another embodiment, the immune disease is celiac disease. In yet still another embodiment, the immune disease is scleroderma. In one embodiment, the immune disease is temporal arteritis. In another embodiment, the immune disease is atopic dermatitis. In yet another embodiment, the immune disease is alopecia areata. In still another embodiment, the immune disease is GVHD. In yet still another embodiment, the immune disease is autoimmune hepatitis. In one embodiment, the immune disease is primary sclerosing cholangitis. In another embodiment, the immune disease is inflammatory myopathy.

### BRIEF DESCRIPTION OF THE DRAWINGS

FIGS. 1A-1C show the ability of exemplary IL-2 muteins to activate pSTAT5 response in primary human Treg cells (CD3+CD4+CD25+FoxP3+) (FIG. 1A), CD8 T cells (CD3+CD8+) (FIG. 1B), or CD4+ Conventional T cells (Tconv) cells (CD3+CD4+CD25−FoxP3−) (FIG. 1C).

FIGS. 2A-2C show the ability of more exemplary IL-2 muteins to activate pSTAT5 response in primary human Treg cells (CD3+CD4+CD25+FoxP3+) (FIG. 2A), CD8 T cells (CD3+CD8+) (FIG. 2B), or Tconv cells (CD3+CD4+CD25−FoxP3−) (FIG. 2C).

FIGS. 3A-3C show the ability of more exemplary IL-2 muteins to activate pSTAT5 response in primary human Treg cells (CD3+CD4+CD25+FoxP3+) (FIG. 2A), CD8 T cells (CD3+CD8+) (FIG. 2B), or Tconv cells (CD3+CD4+CD25−FoxP3−) (FIG. 2C).

FIGS. 4A-4C show the ability of exemplary IL-2 muteins to activate pSTAT5 response in primary rhesus monkey Treg cells (CD3+CD4+CD25+CD127−FoxP3+) (FIG. 4A), CD8 T cells (CD3+CD8+) (FIG. 4B), or Tconv cells (CD3+CD4+CD25−FoxP3−) (FIG. 4C).

FIGS. 5A-5C show the ability of exemplary IL-2 muteins to activate pSTAT5 response in primary rhesus monkey Treg cells (CD3+CD4+CD25+CD127−FoxP3+) (FIG. 5A), CD8 T cells (CD3+CD8+) (FIG. 5B), or Tconv cells (CD3+CD4+CD25−FoxP3−) (FIG. 5C).

FIGS. 6A-6D show human Treg expansion in xeno-GvHD mice treated with exemplary IL-2 muteins. FIG. 6A sets forth results for 21BMT, FIG. 6B sets forth results for DNB558, FIG. 6C sets forth results for 48BMP and FIG. 6D sets forth results for DNB557. Percentage of Treg in human CD4+ T cells in the spleens was quantified by FACS. Fold change in Treg calculated as (% Treg in human CD4 T cells from a treated mouse/mean of % Treg in human CD4 T cells from isotype control group). Concentrations of the muteins in the blood were measured daily and plotted using the Y-axis on the right side. * statistically significant difference comparing to isotype control by 1-way ANOVA with Sidak's multiple comparisons test.

FIGS. 7A-7D show the increase of CD25 expansion on human Tregs in the xeno-GvHD model treated with exemplary IL-2 muteins. FIG. 7A sets forth results for 21BMT, FIG. 7B sets forth results for DNB558, FIG. 7C sets forth results for 48BMP and FIG. 7D sets forth results for DNB557. Mean Fluorescence Intensity (MFI) of CD25 on human CD4⁺ Treg cells in the spleen was quantified by FACS. Fold change is calculated as (MFI of CD25 on human CD4⁺ Treg cells from a treated mouse/mean of MFI of CD25 on human CD4⁺ Treg cells from isotype control group). * statistically significant difference comparing to isotype control by 1-way ANOVA with Sidak's multiple comparisons test.

FIGS. 8A-8D show the concentration (nM) over time and the fold change from baseline in Tregs over time resulting from the PK/PD study set forth in Example 8. FIG. 8A provides results for 21BMT, FIG. 8B provides results for DNB558, FIG. 8C provides results for 48BMP and FIG. 8D provides results for DNB557.

FIGS. 9A-9D are non-limiting schematics showing the orientation of the monovalent and bivalent IL-2 muteins. FIG. 9A depicts a bivalent molecule having the IL-2 mutein linked at its C-terminus to the N terminus of an Fc. FIG. 9B depicts a monovalent molecule having one chain with an IL-2 mutein linked at its C-terminus to the N-terminus of an Fc and the second chain is a Fc portion; the two Fc regions coming together via complementary knob and hole mutations. FIG. 9C depicts a bivalent molecule having an Fc linked at the C-terminus to the N-terminus of the IL-2 mutein. FIG. 9D depicts a monovalent molecule having Fc linked at the C-terminus to the N-terminus of the IL-2 mutein, and the second chain is a Fc portion; the two Fc portions come together via complementary knob and hole mutations.

### DETAILED DESCRIPTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise. Similarly, the plural forms of words include their corresponding singular references unless the context clearly indicates otherwise.

“IL-2 mutein” refers to a molecule comprising a partial or full-length human IL-2 amino acid sequence with one or more amino acid substitutions, deletions, or additions. The term “IL-2 mutein” includes any fusion proteins, protein conjugates, or multi subunit proteins (e.g., dimeric) that comprise a partial or full-length human IL-2 amino acid sequence with one or more amino acid substitutions, deletions, or additions. In certain embodiments, the IL-2 mutein comprises one partial or full-length human IL-2 amino acid sequence with one or more amino acid substitutions, deletions, or additions and thus is referred to as “monovalent” IL-2 mutein. In some embodiments, the IL-2 mutein comprises two partial or full-length human IL-2 amino acid sequences, each with one or more amino acid substitutions, deletions, or additions, and thus is referred to as “bivalent” IL-2 mutein. The full-length wild type human IL-2 amino acid sequence is set forth in SEQ ID NO:39. The partial human IL-2 amino acid sequence comprises at least 50%, 60%, 70%, 80%, 90%, 95%, 96%, 97%, 98%, or 99% of the full-length wild type human IL-2 amino acid sequence as set forth in SEQ ID NO:39.

As used herein, each specific IL-2 mutein, such as “86BCH,” “99BHY,” “43BGO,” “44BGO,” “54BGO,” “47BJO,” “48BJO,” “49BJO,” “65BJO,” “44BJP,” “45BJP,” 46BJP,” or “47BJP,” encompasses a partial or full-length human IL-2 amino acid sequence with its specific amino acid substitutions, deletions, or additions as indicated in Table 1, as well as any fusion proteins, protein conjugates, or multi subunit proteins (e.g., dimeric) that comprise the partial or full-length human IL-2 amino acid sequence with its specific amino acid substitutions, deletions, or additions.

An “Fc” region or domain refers to the heavy chain fragment comprising the C_{H}2 and C_{H}3 domains of an antibody. The antibody can be of any class (e.g., IgG, IgE, IgM, IgD, and IgA) or any subclass (e.g., IgG1, IgG2, IgG3, IgG4, IgA1, and IgA2) of immunoglobulin molecule. Two Fc regions or domains can form a dimer by two or more disulfide bonds and by hydrophobic interactions of the C_{H}3 domains. A “Fc variant” contains one or more amino acid substitutions, deletions, or insertions compared to a wild type Fc region or domain.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. Unless indicated otherwise, as used herein, “binding affinity” refers to intrinsic binding affinity which reflects a 1:1 interaction between members of a binding pair (e.g., an IL-2 mutein and an IL-2 receptor subunit). The ratio of dissociation rate (k_{off}) to association rate (kₒₙ) of an IL-2 mutein to an IL-2 receptor subunit (k_{off}/kₒₙ) is the “dissociation constant” (or “equilibrium dissociation constant” as used interchangeably) KD, which is inversely related to affinity. The lower the KD value, the higher the affinity. The value of KD varies for different complexes of IL-2 mutein and IL-2 receptor subunit and depends on both kₒₙ and k_{off}. The dissociation constant KD for an IL-2 mutein provided herein can be determined using any method provided herein or any other method well known to those skilled in the art, such as surface plasmon resonance (SPR) assay, including but not limited to Biacore and KinExA.

“Administrating” or “administration,” as it applies to an animal, human, subject, cell, tissue, organ, or biological fluid, refers to contact of an exogenous pharmaceutical, therapeutic, diagnostic agent, or composition to the animal, human, subject, cell, tissue, organ, or biological fluid.

The term “subject” includes any organism, preferably an animal, more preferably a mammal (e.g., human, rat, mouse, dog, cat, or rabbit). In a preferred embodiment, the term “subjects” refers to a human. A subject “in need of thereof” is an individual diagnosed with, suspected of having, or predisposed to a disease or disorder which is mediated by IL-2, or a subject for whom prevention of an IL-2 mediated disorder is desired.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

As used herein, the expressions “cell,” “host cell,” “cell line,” and “cell culture” are used interchangeably and all such designations include progeny.

“Treat” or “treatment” means to administer an agent, such as a composition containing any of the IL-2 muteins of the present invention, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting, delaying or slowing the progression of such symptom(s) by any clinically measurable degree. The amount of an agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. The term further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

The terms “prevent,” “preventing,” and “prevention” refer to reducing the likelihood of the onset (or recurrence) of a disease, disorder, condition, or associated symptom(s) (e.g., GVHD, SLE, CLE, multiple sclerosis, ulcerative colitis, or Crohn's disease).

An “IL-2-mediated disease,” “IL-2-mediated disorder,” and “IL-2-mediated condition” are used interchangeably and refer to any disease, disorder, or condition that is completely or partially caused by or is the result of IL-2 signaling and/or alternatively any disease, disorder, or condition in which it is desirable to modulate IL-2 signaling, either systematically or in selected cell types, tissues, or organs.

The term “therapeutically effective amount” as used herein refers to the amount of an agent (e.g., an IL-2 muteins provided herein or any other agent described herein) that is sufficient to reduce and/or ameliorate the severity and/or duration of a given disease, disorder, or condition, and/or a symptom related thereto (e.g., GVHD, SLE, CLE, multiple sclerosis, ulcerative colitis, or Crohn's disease). A “therapeutically effective amount” of a substance/molecule/agent of the present disclosure (e.g., an IL-2 mutein) may vary according to factors such as the disease state, age, sex, and weight of the individual, and the ability of the substance/molecule/agent to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of an IL-2 mutein or other agent (e.g., drug) effective to “treat” a disease, disorder, or condition, in a subject or mammal.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The term “carrier” can also refer to a diluent, adjuvant (e.g., Freund's adjuvant (complete or incomplete)), excipient, or vehicle. Such carriers, including pharmaceutical carriers, can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington and Gennaro, Remington's Pharmaceutical Sciences (18th ed. 1990). Compositions, including pharmaceutical compounds, may contain an IL-2 mutein, for example, in isolated or purified form, together with a suitable number of carriers.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

An “isolated nucleic acid” is a nucleic acid, for example, an RNA, DNA, or a mixed nucleic acid, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an IL-2 mutein as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an IL-2 mutein of the present disclosure may include a host cell into which nucleic acids encoding the IL-2 mutein have been introduced. Suitable host cells are disclosed below.

### IL-2 Muteins

The invention provides IL-2 muteins that bind to the IL-2 receptor α subunit but do not have measurable binding to the IL-2 receptor β subunit.

The various IL-2 muteins described herein can be a partial or full-length human IL-2 molecule comprising one or more amino acid substitutions, deletions, or additions relative to an IL-2 reference amino acid sequence, e.g. the wild-type amino acid sequence set forth in SEQ ID NO:39. In some embodiments, the partial or full-length human IL-2 molecule comprising one or more amino acid substitutions, deletions, or additions is fused to another polypeptide, such as a Fc region of a human immunoglobulin (e.g., IgG1, IgG2, IgG3, or IgG4). The Fc region can be a wild type Fc or a Fc variant with desired characteristics or properties, such as modified serum half-life, complement fixation, Fc receptor binding, and/or effector function (e.g., antigen-dependent cellular cytotoxicity).

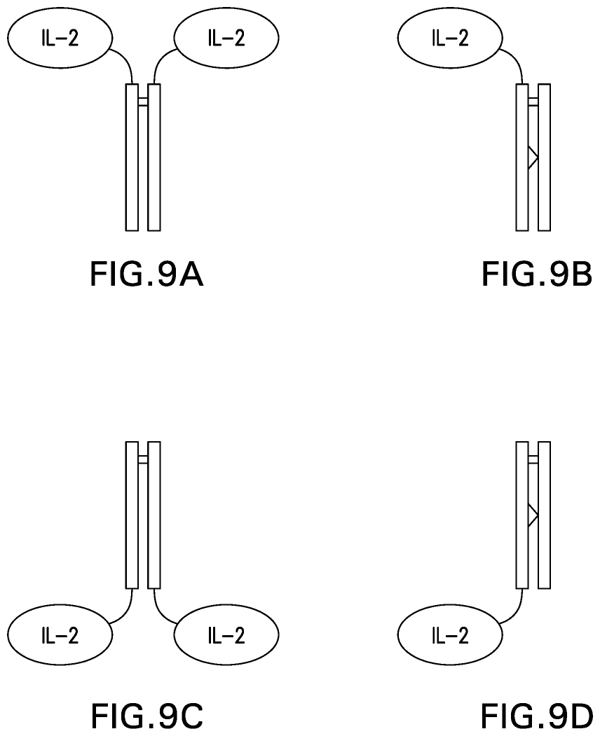

In one embodiment, the Fc region is modified with two amino acid substitutions, L234A and L235A (sometimes referred to as “LALA” mutations) relative to the wildtype Fc polypeptide sequence that reduce/ablate effector function. In another embodiment, the Fc region is modified with three amino acid substitutions, L234A, L235A and D265S that reduce/ablate effector function (sometimes referred to as “LALADS” mutations). In yet another embodiment, the Fc region is modified with three amino acid substitutions, M252Y, S254T, and T256E that increase half-life in serum (sometimes referred to as “YTE” mutations). In still another embodiment, the Fc variant have a combination of different mutations described herein, for example, LALA and YTE mutations, or LALADS and YTE mutations. The partial or full-length human IL-2 molecule comprising one or more substitutions, deletions, or additions fused with a Fc variant can form homodimers through the Fc variant. Such homodimers contain two IL-2 mutant molecules and are thus bivalent IL-2 muteins. FIGS. 9A and 9C set forth a non-limiting schematic of bivalent IL-2 muteins.

In other embodiments, a pair of Fc variants include one Fc region comprising a knob mutation (“Fc knob”) and another Fc region comprising a hole mutation (“Fc hole”). The Fc knob and the Fc hole can form a heterodimer (sometimes referred to as “knob in hole” mutations). In certain embodiments, the Fc region is modified with two amino acid substitutions, S354C and T366W (sometimes referred to as “knob” mutations). In other embodiments, the Fc region is modified with four amino acid substitutions, Y349C, T366S, L368A, and Y407V (sometimes referred to as “hole” mutations). In certain embodiments, the Fc region is modified with the four amino acid substitutions referred to as “hole” mutations and further contains two additional amino acid substitutions H435R and Y435F. The partial or full-length human IL-2 molecule comprising one or more substitutions, deletions, or additions can be fused with Fc knob or Fc hole. In some embodiments, the partial or full-length human IL-2 molecule comprising one or more substitutions, deletions, or additions fused with Fe knob is paired with Fc hole to form a monovalent IL-2 mutein. In certain embodiments, the partial or full-length human IL-2 molecule comprising one or more substitutions, deletions, or additions fused with Fc hole is paired with Fc knob to form a monovalent IL-2 mutein. FIGS. 9B and 9D set forth a non-limiting schematic of a monovalent IL-2 mutein. In other embodiments, the partial or full-length human IL-2 molecule comprising one or more substitutions, deletions, or additions is fused with Fc without either knob or hole, and the Fc-fused IL-2 molecule can form a homodimer bivalent IL-2 mutein (see, e.g., FIGS. 9A and 9C as non-limiting examples of bivalent IL-2 muteins).

The partial or full-length human IL-2 molecule comprising one or more substitutions, deletions, or additions can be fused to the N-terminus or C-terminus of an Fc variant, through a variety of peptide linkers between the IL-2 molecule and the Fc variant. In one embodiment, the partial or full-length human IL-2 molecule comprising one or more substitutions, deletions, or additions is fused to the N-terminus of an Fc variant, and the peptide linker is between the C-terminus of the IL-2 molecule and the N-terminus of the Fc variant. In another embodiment, the partial or full-length human IL-2 molecule comprising one or more substitutions, deletions, or additions is fused to the C-terminus of an Fc variant, and the peptide linker is between the C-terminus of the Fc variant and the N-terminus of the IL-2 molecule.

In certain embodiments, provided is an IL-2 mutein that comprises a first polypeptide comprising an amino acid sequence as set forth in SEQ ID NO:1, 2, or 40 in which the D at position 19 of SEQ ID NO: 1, 2, or 40 is substituted with an N and the P at position 33 of SEQ ID NO:1, 2, or 40 is substituted with an R. In one embodiment, the IL-2 mutein comprises a first polypeptide comprising an amino acid sequence as set forth in SEQ ID NO:1 in which the D at position 19 of SEQ ID NO:1 is substituted with an N and the P at position 33 of SEQ ID NO:1 is substituted with an R. In another embodiment, the IL-2 mutein comprises a first polypeptide comprising an amino acid sequence as set forth in SEQ ID NO:2 in which the D at position 19 of SEQ ID NO:2 is substituted with an N and the P at position 33 of SEQ ID NO:2 is substituted with an R. In yet another embodiment, the IL-2 mutein comprises a first polypeptide comprising an amino acid sequence as set forth in SEQ ID NO:40 in which the D at position 19 of SEQ ID NO:40 is substituted with an N and the P at position 33 of SEQ ID NO:40 is substituted with an R.

In some embodiments, provided is an IL-2 mutein that comprises a first polypeptide comprising an amino acid sequence as set forth in SEQ ID NO:1, 2, or 40 in which the D at position 19 of SEQ ID NO: 1, 2, or 40 and the E at position 67 of SEQ ID NO:1, 2, or 40 is substituted with an S. In one embodiment, the IL-2 mutein comprises a first polypeptide comprising an amino acid sequence as set forth in SEQ ID NO:1 in which the D at position 19 of SEQ ID NO:1 is substituted with an N and the E at position 67 of SEQ ID NO:1 is substituted with an S. In another embodiment, the IL-2 mutein comprises a first polypeptide comprising an amino acid sequence as set forth in SEQ ID NO:2 in which the D at position 19 of SEQ ID NO:2 is substituted with an N and the E at position 67 of SEQ ID NO:2 is substituted with an S. In yet another embodiment, the IL-2 mutein comprises a first polypeptide comprising an amino acid sequence as set forth in SEQ ID NO:40 in which the D at position 19 of SEQ ID NO:40 is substituted with an N and the E at position 67 of SEQ ID NO:40 is substituted with an S.

In other embodiments, provided is an IL-2 mutein that comprises a first polypeptide comprising an amino acid sequence as set forth in SEQ ID NO:1, 2, or 40 in which the D at position 19 of SEQ ID NO:1, 2, or 40 is substituted with an N, the P at position 33 of SEQ ID NO:1, 2, or 40 is substituted with an R, and the E at position 67 of SEQ ID NO:1, 2, or 40 is substituted with an S. In one embodiment, the IL-2 mutein comprises a first polypeptide comprising an amino acid sequence as set forth in SEQ ID NO:1 in which the D at position 19 of SEQ ID NO:1 is substituted with an N, the P at position 33 of SEQ ID NO:1 is substituted with an R, and the E at position 67 of SEQ ID NO:1 is substituted with an S. In another embodiment, the IL-2 mutein comprises a first polypeptide comprising an amino acid sequence as set forth in SEQ ID NO:2 in which the D at position 19 of SEQ ID NO:2 is substituted with an N, the P at position 33 of SEQ ID NO:2 is substituted with an R, and the E at position 67 of SEQ ID NO:2 is substituted with an S In yet another embodiment, the IL-2 mutein comprises a first polypeptide comprising an amino acid sequence as set forth in SEQ ID NO:40 in which the D at position 19 of SEQ ID NO:40 is substituted with an N, the P at position 33 of SEQ ID NO: 40 is substituted with an R, and the E at position 67 of SEQ ID NO:40 is substituted with an S.

In yet other embodiments of the IL-2 muteins, the first polypeptide further comprises a V to A substitution at position 68, an N to R substitution at position 70, or a Q to P substitution at position 73 of SEQ ID NO:1, 2, or 40.

In still other embodiments of the IL-2 muteins, the first polypeptide further comprises any two of the three following substitutions: a V to A substitution at position 68, an N to R substitution at position 70, or a Q to P substitution at position 73 of SEQ ID NO:1, 2, or 40.

In yet still other embodiments of the IL-2 muteins, the first polypeptide further comprises a V to A substitution at position 68, an N to R substitution at position 70, and a Q to P substitution at position 73 of SEQ ID NO:1, 2, or 40.

In one embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, and the V at position 68 of SEQ ID NO:1 is substituted with an A. In another embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, and the N at position 70 of SEQ ID NO:1 is substituted with an R. In yet another embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, and the Q at position 73 of SEQ ID NO:1 is substituted with a P. In still another embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, the V at position 68 of SEQ ID NO:1 is substituted with an A, and the N at position 70 of SEQ ID NO:1 is substituted with an R. In one embodiment, the IL-2 mutein comprises a first polypeptide comprising the amino acid sequence as set forth in SEQ ID NO:1 in which the D at position 19 is substituted with an N, the P at position 33 of SEQ ID NO:1 is substituted with an R, the V at position 68 of SEQ ID NO:1 is substituted with an A, and the Q at position 73 of SEQ ID NO:1 is substituted with a P. In another embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, the N at position 70 of SEQ ID NO:1 is substituted with an R, and the Q at position 73 of SEQ ID NO:1 is substituted with a P. In yet another embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, the V at position 68 of SEQ ID NO:1 is substituted with an A, the N at position 70 of SEQ ID NO:1 is substituted with an R, and the Q at position 73 of SEQ ID NO:1 is substituted with a P.

In one embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, and the V at position 68 of SEQ ID NO:2 is substituted with an A. In another embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, and the N at position 70 of SEQ ID NO:2 is substituted with an R. In yet another embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, and the Q at position 73 of SEQ ID NO:2 is substituted with a P. In still another embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, the V at position 68 of SEQ ID NO:2 is substituted with an A, and the N at position 70 of SEQ ID NO:2 is substituted with an R. In one embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, the V at position 68 of SEQ ID NO:2 is substituted with an A, and the Q at position 73 of SEQ ID NO:2 is substituted with a P. In another embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, the N at position 70 of SEQ ID NO:2 is substituted with an R, and the Q at position 73 of SEQ ID NO:2 is substituted with a P. In yet another embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, the V at position 68 of SEQ ID NO:2 is substituted with an A, the N at position 70 of SEQ ID NO:2 is substituted with an R, and the Q at position 73 of SEQ ID NO:2 is substituted with a P.

In one embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, and the V at position 68 of SEQ ID NO:40 is substituted with an A. In another embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, and the N at position 70 of SEQ ID NO:40 is substituted with an R. In yet another embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, and the Q at position 73 of SEQ ID NO:40 is substituted with a P. In still another embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, the V at position 68 of SEQ ID NO:40 is substituted with an A, and the N at position 70 of SEQ ID NO:40 is substituted with an R. In one embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, the V at position 68 of SEQ ID NO:40 is substituted with an A, and the Q at position 73 of SEQ ID NO:40 is substituted with a P. In another embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, the N at position 70 of SEQ ID NO:40 is substituted with an R, and the Q at position 73 of SEQ ID NO:40 is substituted with a P. In yet another embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, the V at position 68 of SEQ ID NO:40 is substituted with an A, the N at position 70 of SEQ ID NO:40 is substituted with an R, and the Q at position 73 of SEQ ID NO:40 is substituted with a P.

In one embodiment, the IL-2 mutein comprises a first polypeptide comprising the amino acid sequence as set forth in SEQ ID NO:1 in which the D at position 19 of SEQ ID NO:1 is substituted with an N, the E at position 67 of SEQ ID NO:1 is substituted with an S, and the V at position 68 of SEQ ID NO:1 is substituted with an A. In another embodiment, the IL-2 mutein comprises a first polypeptide comprising the amino acid sequence as set forth in SEQ ID NO:1 in which the D at position 19 of SEQ ID NO:1 is substituted with an N, the E at position 67 of SEQ ID NO:1 is substituted with an S, and the N at position 70 of SEQ ID NO:1 is substituted with an R. In yet another embodiment, the IL-2 mutein comprises a first polypeptide comprising an amino acid sequence as set forth in SEQ ID NO:1 in which the D at position 19 of SEQ ID NO:1 is substituted with an N, the E at position 67 of SEQ ID NO:1 is substituted with an S, and the Q at position 73 of SEQ ID NO:1 is substituted with a P. In still another embodiment, the IL-2 mutein comprises a first polypeptide comprising the amino acid sequence as set forth in SEQ ID NO:1 in which the D at position 19 of SEQ ID NO:1 is substituted with an N, the E at position 67 of SEQ ID NO:1 is substituted with an S, the V at position 68 of SEQ ID NO:1 is substituted with an A, and the N at position 70 of SEQ ID NO:1 is substituted with an R. In one embodiment, the IL-2 mutein comprises a first polypeptide comprising the amino acid sequence as set forth in SEQ ID NO:1 in which the D at position 19 of SEQ ID NO:1 is substituted with an N, the E at position 67 of SEQ ID NO:1 is substituted with an S, the V at position 68 of SEQ ID NO:1 is substituted with an A, and the Q at position 73 of SEQ ID NO:1 is substituted with a P. In another embodiment, the IL-2 mutein comprises a first polypeptide comprising the amino acid sequence as set forth in SEQ ID NO:1 in which the D at position 19 of SEQ ID NO:1 is substituted with an N, the E at position 67 of SEQ ID NO:1 is substituted with an S, the N at position 70 of SEQ ID NO:1 is substituted with an R, and the Q at position 73 of SEQ ID NO:1 is substituted with a P. In yet another embodiment, the IL-2 mutein comprises a first polypeptide comprising the amino acid sequence as set forth in SEQ ID NO:1 in which the D at position 19 of SEQ ID NO:1 is substituted with an N, the E at position 67 of SEQ ID NO:1 is substituted with an S, the V at position 68 of SEQ ID NO:1 is substituted with an A, the N at position 70 of SEQ ID NO:1 is substituted with an R, and the Q at position 73 of SEQ ID NO:1 is substituted with a P.

In one embodiment, the IL-2 mutein comprises a first polypeptide comprising the amino acid sequence as set forth in SEQ ID NO:2 in which the D at position 19 of SEQ ID NO:2 is substituted with an N, the E at position 67 of SEQ ID NO:2 is substituted with an S, and the V at position 68 of SEQ ID NO:2 is substituted with an A. In another embodiment, the IL-2 mutein comprises a first polypeptide comprising the amino acid sequence as set forth in SEQ ID NO:2 in which the D at position 19 of SEQ ID NO:2 is substituted with an N, the E at position 67 of SEQ ID NO:2 is substituted with an S, and the N at position 70 of SEQ ID NO:2 is substituted with an R. In yet another embodiment, the IL-2 mutein comprises a first polypeptide comprising the amino acid sequence as set forth in SEQ ID NO:2 in which the D at position 19 of SEQ ID NO:2 is substituted with an N, the E at position 67 of SEQ ID NO:2 is substituted with an S, and the Q at position 73 of SEQ ID NO:2 is substituted with a P. In still another embodiment, the IL-2 mutein comprises a first polypeptide comprising the amino acid sequence as set forth in SEQ ID NO:2 in which the D at position 19 of SEQ ID NO:2 is substituted with an N, the E at position 67 of SEQ ID NO:2 is substituted with an S, the V at position 68 of SEQ ID NO:2 is substituted with an A, and the N at position 70 of SEQ ID NO:2 is substituted with an R. In one embodiment, the IL-2 mutein comprises a first polypeptide comprising the amino acid sequence as set forth in SEQ ID NO:2 in which the D at position 19 of SEQ ID NO:2 is substituted with an N, the E at position 67 of SEQ ID NO:2 is substituted with an S, the V at position 68 of SEQ ID NO:2 is substituted with an A, and the Q at position 73 of SEQ ID NO:2 is substituted with a P. In another embodiment, the IL-2 mutein comprises a first polypeptide comprising the amino acid sequence as set forth in SEQ ID NO:2 in which the D at position 19 of SEQ ID NO:2 is substituted with an A, the E at position 67 of SEQ ID NO:2 is substituted with an S, the N at position 70 of SEQ ID NO:2 is substituted with an R, and the Q at position 73 of SEQ ID NO:2 is substituted with a P. In yet another embodiment, the IL-2 mutein comprises a first polypeptide comprising the amino acid sequence as set forth in SEQ ID NO:2 in which the D at position 19 of SEQ ID NO:2 is substituted with an N, the E at position 67 of SEQ ID NO:2 is substituted with an S, the V at position 68 of SEQ ID NO:2 is substituted with an A, the N at position 70 of SEQ ID NO:2 is substituted with an R, and the Q at position 73 of SEQ ID NO:2 is substituted with a P.

In one embodiment, the IL-2 mutein comprises a first polypeptide comprising the amino acid sequence as set forth in SEQ ID NO:40 in which the D at position 19 of SEQ ID NO:40 is substituted with an N, the E at position 67 of SEQ ID NO:40 is substituted with an S, and the V at position 68 of SEQ ID NO:40 is substituted with an A. In another embodiment, the IL-2 mutein comprises a first polypeptide comprising the amino acid sequence as set forth in SEQ ID NO:40 in which the D at position 19 of SEQ ID NO:40 is substituted with an N, the E at position 67 of SEQ ID NO:40 is substituted with an S, and the N at position 70 of SEQ ID NO:40 is substituted with an R. In yet another embodiment, the IL-2 mutein comprises a first polypeptide comprising the amino acid sequence as set forth in SEQ ID NO:40 in which the D at position 19 of SEQ ID NO:40 is substituted with an N, the E at position 67 of SEQ ID NO:40 is substituted with an S, and the Q at position 73 of SEQ ID NO:40 is substituted with a P. In still another embodiment, the IL-2 mutein comprises a first polypeptide comprising the amino acid sequence as set forth in SEQ ID NO:40 in which the D at position 19 of SEQ ID NO:40 is substituted with an N, the E at position 67 of SEQ ID NO:40 is substituted with an S, the V at position 68 of SEQ ID NO:40 is substituted with an A, and the N at position 70 of SEQ ID NO:40 is substituted with an R. In one embodiment, the IL-2 mutein comprises a first polypeptide comprising the amino acid sequence as set forth in SEQ ID NO:40 in which the D at position 19 of SEQ ID NO:40 is substituted with an N, the E at position 67 of SEQ ID NO:40 is substituted with an S, the V at position 68 of SEQ ID NO:40 is substituted with an A, and the Q at position 73 of SEQ ID NO:40 is substituted with a P. In another embodiment, the IL-2 mutein comprises a first polypeptide comprising the amino acid sequence as set forth in SEQ ID NO:40 in which the D at position 19 of SEQ ID NO:40 is substituted with an N, the E at position 67 of SEQ ID NO:40 is substituted with an S, the N at position 70 of SEQ ID NO:40 is substituted with an R, and the Q at position 73 of SEQ ID NO:40 is substituted with a P. In yet another embodiment, the IL-2 mutein comprises a first polypeptide comprising the amino acid sequence as set forth in SEQ ID NO:40 in which the D at position 19 of SEQ ID NO:40 is substituted with an N, the E at position 67 of SEQ ID NO:40 is substituted with an S, the V at position 68 of SEQ ID NO:40 is substituted with an A, the N at position 70 of SEQ ID NO:40 is substituted with an R, and the Q at position 73 of SEQ ID NO:40 is substituted with a P.

In one embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, the E at position 67 of SEQ ID NO:1 is substituted with an S, and the V at position 68 of SEQ ID NO:1 is substituted with an A. In another embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, the E at position 67 of SEQ ID NO:1 is substituted with an S, and the N at position 70 of SEQ ID NO:1 is substituted with an R. In yet another embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, the E at position 67 of SEQ ID NO:1 is substituted with an S, and the Q at position 73 of SEQ ID NO:1 is substituted with a P. In still another embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, the E at position 67 of SEQ ID NO:1 is substituted with an S, the V at position 68 of SEQ ID NO:1 is substituted with an A, and the N at position 70 of SEQ ID NO:1 is substituted with an R. In one embodiment, the IL-2 mutein comprises a first polypeptide comprising the amino acid sequence as set forth in SEQ ID NO:1 in which the D at position 19 is substituted with an N, the P at position 33 of SEQ ID NO:1 is substituted with an R, the E at position 67 of SEQ ID NO:1 is substituted with an S, the V at position 68 of SEQ ID NO:1 is substituted with an A, and the Q at position 73 of SEQ ID NO:1 is substituted with a P. In another embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, the E at position 67 of SEQ ID NO:1 is substituted with an S, the N at position 70 of SEQ ID NO:1 is substituted with an R, and the Q at position 73 of SEQ ID NO:1 is substituted with a P. In yet another embodiment, the IL-2 mutein comprises a first polypeptide comprising the amino acid sequence as set forth in SEQ ID NO:1 in which the D at position 19 of SEQ ID NO:1 is substituted with an N, the P at position 33 of SEQ ID NO:1 is substituted with an R, the E at position 67 of SEQ ID NO:1 is substituted with an S, the V at position 68 of SEQ ID NO:1 is substituted with an A, the N at position 70 of SEQ ID NO:1 is substituted with an R, and the Q at position 73 of SEQ ID NO:1 is substituted with a P.

In one embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, the E at position 67 of SEQ ID NO:2 is substituted with an S, and the V at position 68 of SEQ ID NO:2 is substituted with an A. In another embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, the E at position 67 of SEQ ID NO:2 is substituted with an R, and the N at position 70 of SEQ ID NO:2 is substituted with an R. In yet another embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, the E at position 67 of SEQ ID NO:2 is substituted with an S, and the Q at position 73 of SEQ ID NO:2 is substituted with a P. In still another embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, the E at position 67 of SEQ ID NO:2 is substituted with an S, the V at position 68 of SEQ ID NO:2 is substituted with an A, and the N at position 70 of SEQ ID NO:2 is substituted with an R. In one embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, the E at position 67 of SEQ ID NO:2 is substituted with an S, the V at position 68 of SEQ ID NO:2 is substituted with an A, and the Q at position 73 of SEQ ID NO:2. Is substituted with a P In another embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, the E at position 67 of SEQ ID NO:2 is substituted with an S, the N at position 70 of SEQ ID NO:2 is substituted with an R, and the Q at position 73 of SEQ ID NO:2 is substituted with a P. In yet another embodiment, the IL-2 mutein comprises a first polypeptide comprising the amino acid sequence as set forth in SEQ ID NO:2 in which the D at position 19 of SEQ ID NO:2 is substituted with an N, the P at position 33 of SEQ ID NO:2 is substituted with an R, the E at position 67 of SEQ ID NO:2 is substituted with an S, the V at position 68 of SEQ ID NO:2 is substituted with an A, the N at position 70 of SEQ ID NO:2 is substituted with an R, and the Q at position 73 of SEQ ID NO:2 is substituted with a P.

In one embodiment, the IL-2 mutein comprises a first polypeptide comprising the amino acid sequence as set forth in SEQ ID NO:40 in which the D at position 19 of SEQ ID NO:40 is substituted with an N, a P at position 33 of SEQ ID NO:40 is substituted with an R, an E at position 67 of SEQ ID NO:40 is substituted with an S, and the V at position 68 of SEQ ID NO:40 is substituted with an A. In another embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, the E at position 67 of SEQ ID NO:40 is substituted with an S, and the N at position 70 of SEQ ID NO:40. In yet another embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, the E at position 67 of SEQ ID NO:40 is substituted with an S, and the Q at position 73 of SEQ ID NO:40 is substituted with a P. In still another embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, the E at position 67 of SEQ ID NO:40 is substituted with an S, the V at position 68 is substituted with an A, and the N at position 70 of SEQ ID NO:40 is substituted with an R. In one embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, the E at position 67 of SEQ ID NO:40 is substituted with an S, the V at position 68 of SEQ ID NO:40 is substituted with an A, and the Q at position 73 of SEQ ID NO:40 is substituted with a P. In another embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, the E at position 67 of SEQ ID NO:40 is substituted with an S, the N at position 70 of SEQ ID NO:40 is substituted with an R, and the Q at position 73 of SEQ ID NO:40 is substituted with a P. In yet another embodiment, the IL-2 mutein comprises a first polypeptide comprising the amino acid sequence as set forth in SEQ ID NO:40 in which the D at position 19 of SEQ ID NO:40 is substituted with an N, the P at position 33 of SEQ ID NO:40 is substituted with an R, the E at position 67 of SEQ ID NO:40 is substituted with an S, the V at position 68 of SEQ ID NO:40 is substituted with an A, the N at position 70 of SEQ ID NO:40 is substituted with an R, and the Q at position 73 of SEQ ID NO:40 is substituted with a P.

In one embodiment, the IL-2 mutein comprises a first polypeptide comprising the amino acid sequence as set forth in SEQ ID NO:1 in which the D at position 19 of SEQ ID NO:1 is substituted with an N, the V at position 68 of SEQ ID NO:1 is substituted with an A, and the Q at position 73 of SEQ ID NO:1 is substituted with a P. In one embodiment, the IL-2 mutein comprises a first polypeptide comprising the amino acid sequence as set forth in SEQ ID NO:2 in which the D at position 19 of SEQ ID NO:2 is substituted with an N, the V at position 68 of SEQ ID NO:2 is substituted with an A, and the Q at position 73 of SEQ ID NO:2 is substituted with a P. In one embodiment, the IL-2 mutein comprises a first polypeptide comprising the amino acid sequence as set forth in SEQ ID NO:40 in which the D at position 19 of SEQ ID NO:40 is substituted with an N, the V at position 68 of SEQ ID NO:40 is substituted with an A, and the Q at position 73 of SEQ ID NO:40 is substituted with a P. In certain embodiments, the IL-2 mutein comprises a first polypeptide comprising the amino acid sequence as set forth in SEQ ID NO:3, 4, 5, 6, 7, 8, 9, 10, 11, or 12. In one embodiment, the IL-2 mutein comprises a first polypeptide comprising the amino acid sequence as set forth in SEQ ID NO:3. In another embodiment, the IL-2 mutein comprises a first polypeptide comprising the amino acid sequence as set forth in SEQ ID NO:4. In yet another embodiment, the IL-2 mutein comprises a first polypeptide comprising the amino acid sequence as set forth in SEQ ID NO:5. In still another embodiment, the IL-2 mutein comprises a first polypeptide comprising the amino acid sequence as set forth in SEQ ID NO:6. In yet still another embodiment, the IL-2 mutein comprises a first polypeptide comprising the amino acid sequence as set forth in SEQ ID NO:7. In one embodiment, the IL-2 mutein comprises a first polypeptide comprising the amino acid sequence as set forth in SEQ ID NO:8. In another embodiment, the IL-2 mutein comprises a first polypeptide comprising the amino acid sequence as set forth in SEQ ID NO:9. In yet another embodiment, the IL-2 mutein comprises a first polypeptide comprising the amino acid sequence as set forth in SEQ ID NO:10. In still another embodiment, the IL-2 mutein comprises a first polypeptide comprising the amino acid sequence as set forth in SEQ ID NO:11. In yet still another embodiment, the IL-2 mutein comprises a first polypeptide comprising the amino acid sequence as set forth in SEQ ID NO:12.

In some embodiments of the IL-2 muteins, the first polypeptide further comprises the amino acid sequence as set forth in SEQ ID NO:13, 14, or 15. In one embodiment, the IL-2 mutein comprises a first polypeptide comprising the amino acid sequence as set forth in SEQ ID NO:3, 4, 5, 6, 7, 8, 9, 10, 11, or 12 and further comprising the amino acid sequence as set forth in SEQ ID NO:13. In another embodiment, the IL-2 mutein comprises a first polypeptide comprising the amino acid sequence as set forth in SEQ ID NO:3, 4, 5, 6, 7, 8, 9, 10, 11, or 12 and further comprising the amino acid sequence as set forth in SEQ ID NO:14. In yet another embodiment, the IL-2 mutein comprises a first polypeptide comprising the amino acid sequence as set forth in SEQ ID NO:3, 4, 5, 6, 7, 8, 9, 10, 11, or 12 and further comprising the amino acid sequence as set forth in SEQ ID NO:15.

In one embodiment, the IL-2 mutein comprises a first polypeptide comprising the amino acid sequence as set forth in SEQ ID NO:3 and the amino acid sequence as set forth in SEQ ID NO:13. In another embodiment, the IL-2 mutein comprises a first polypeptide comprising the amino acid sequence as set forth in SEQ ID NO:4 and the amino acid sequence as set forth in SEQ ID NO: 13. In yet another embodiment, the IL-2 mutein comprises a first polypeptide comprising the amino acid sequence as set forth in SEQ ID NO:5 and the amino acid sequence as set forth in SEQ ID NO: 13. In still another embodiment, the IL-2 mutein comprises a first polypeptide comprising the amino acid sequence as set forth in SEQ ID NO:6 and the amino acid sequence as set forth in SEQ ID NO:13. In yet still another embodiment, the IL-2 mutein comprises a first polypeptide comprising the amino acid sequence as set forth in SEQ ID NO:7 and the amino acid sequence as set forth in SEQ ID NO: 13. In one embodiment, the IL-2 mutein comprises a first polypeptide comprising the amino acid sequence as set forth in SEQ ID NO:8 and the amino acid sequence as set forth in SEQ ID NO:13. In another embodiment, the IL-2 mutein comprises a first polypeptide comprising the amino acid sequence as set forth in SEQ ID NO:9 and the amino acid sequence as set forth in SEQ ID NO:13. In yet another embodiment, the IL-2 mutein comprises a first polypeptide comprising the amino acid sequence as set forth in SEQ ID NO:10 and the amino acid sequence as set forth in SEQ ID NO:13. In still another embodiment, the IL-2 mutein comprises a first polypeptide comprising the amino acid sequence as set forth in SEQ ID NO:11 and the amino acid sequence as set forth in SEQ ID NO:13. In yet still another embodiment, the IL-2 mutein comprises a first polypeptide comprising the amino acid sequence as set forth in SEQ ID NO:12 and the amino acid sequence as set forth in SEQ ID NO:13.

In one embodiment, the IL-2 mutein comprises a first polypeptide comprising the amino acid sequence as set forth in SEQ ID NO:3 and the amino acid sequence as set forth in SEQ ID NO:14. In another embodiment, the IL-2 mutein comprises a first polypeptide comprising the amino acid sequence as set forth in SEQ ID NO:4 and the amino acid sequence as set forth in SEQ ID NO: 14. In yet another embodiment, the IL-2 mutein comprises a first polypeptide comprising the amino acid sequence as set forth in SEQ ID NO:5 and the amino acid sequence as set forth in SEQ ID NO:14. In still another embodiment, the IL-2 mutein comprises a first polypeptide comprising the amino acid sequence as set forth in SEQ ID NO:6 and the amino acid sequence as set forth in SEQ ID NO:14. In yet still another embodiment, the IL-2 mutein comprises a first polypeptide comprising the amino acid sequence as set forth in SEQ ID NO:7 and the amino acid sequence as set forth in SEQ ID NO: 14. In one embodiment, the IL-2 mutein comprises a first polypeptide comprising the amino acid sequence as set forth in SEQ ID NO:8 and the amino acid sequence as set forth in SEQ ID NO:14. In another embodiment, the IL-2 mutein comprises a first polypeptide comprising the amino acid sequence as set forth in SEQ ID NO:9 and the amino acid sequence as set forth in SEQ ID NO:14. In yet another embodiment, the IL-2 mutein comprises a first polypeptide comprising the amino acid sequence as set forth in SEQ ID NO:10 and the amino acid sequence as set forth in SEQ ID NO:14. In still another embodiment, the IL-2 mutein comprises a first polypeptide comprising the amino acid sequence as set forth in SEQ ID NO:11 and the amino acid sequence as set forth in SEQ ID NO:14. In yet still another embodiment, the IL-2 mutein comprises a first polypeptide comprising the amino acid sequence as set forth in SEQ ID NO:12 and the amino acid sequence as set forth in SEQ ID NO:14.

In one embodiment, the IL-2 mutein comprises a first polypeptide comprising the amino acid sequence as set forth in SEQ ID NO:3 and the amino acid sequence as set forth in SEQ ID NO:15. In another embodiment, the IL-2 mutein comprises a first polypeptide comprising the amino acid sequence as set forth in SEQ ID NO:4 and the amino acid sequence as set forth in SEQ ID NO: 15. In yet another embodiment, the IL-2 mutein comprises a first polypeptide comprising the amino acid sequence as set forth in SEQ ID NO:5 and the amino acid sequence as set forth in SEQ ID NO:15. In still another embodiment, the IL-2 mutein comprises a first polypeptide comprising the amino acid sequence as set forth in SEQ ID NO:6 and the amino acid sequence as set forth in SEQ ID NO:15. In yet still another embodiment, the IL-2 mutein comprises a first polypeptide comprising the amino acid sequence as set forth in SEQ ID NO:7 and the amino acid sequence as set forth in SEQ ID NO: 15. In one embodiment, the IL-2 mutein comprises a first polypeptide comprising the amino acid sequence as set forth in SEQ ID NO:8 and the amino acid sequence as set forth in SEQ ID NO: 15. In another embodiment, the IL-2 mutein comprises a first polypeptide comprising the amino acid sequence as set forth in SEQ ID NO:9 and the amino acid sequence as set forth in SEQ ID NO:15. In yet another embodiment, the IL-2 mutein comprises a first polypeptide comprising the amino acid sequence as set forth in SEQ ID NO:10 and the amino acid sequence as set forth in SEQ ID NO:15. In still another embodiment, the IL-2 mutein comprises a first polypeptide comprising the amino acid sequence as set forth in SEQ ID NO:11 and the amino acid sequence as set forth in SEQ ID NO:15. In yet still another embodiment, the IL-2 mutein comprises a first polypeptide comprising the amino acid sequence as set forth in SEQ ID NO:12 and the amino acid sequence as set forth in SEQ ID NO:15.

In other embodiments of the IL-2 muteins, the first polypeptide further comprises a linker as set forth in SEQ ID NO:16, 41, or 42. In one embodiment, the first polypeptide further comprises a linker as set forth in SEQ ID NO:16. In another embodiment, the first polypeptide further comprises a linker as set forth in SEQ ID NO:41. In yet another embodiment, the first polypeptide further comprises a linker as set forth in SEQ ID NO:42. In some embodiments, the first amino acid sequence as set forth in SEQ ID NO:3, 4, 5, 6, 7, 8, 9, 10, 11, or 12 is fused to the N-terminus of the second amino acid sequence as set forth in SEQ ID NO:13, 14, or 15, and the linker as set forth in SEQ ID NO:16, 41, or 42 is between the C-terminus of the first amino acid sequence and the N-terminus of the second amino acid sequence. In another embodiment, the first amino acid sequence as set forth in SEQ ID NO:3, 4, 5, 6, 7, 8, 9, 10, 11, or 12 is fused to the C-terminus of the second amino acid sequence as set forth in SEQ ID NO:13, 14, or 15, and the peptide linker is between the C-terminus of the second amino acid sequence and the N-terminus of the first amino acid sequence.

In one embodiment, the IL-2 mutein comprises a first polypeptide comprising the amino acid sequence as set forth in SEQ ID NO:3, 4, 5, 6, 7, 8, 9, 10, 11, or 12 and further comprising the amino acid sequence as set forth in SEQ ID NO:13 and a linker as set forth in SEQ ID NO:16. In another embodiment, the IL-2 mutein comprises a first polypeptide comprising the amino acid sequence as set forth in SEQ ID NO:3, 4, 5, 6, 7, 8, 9, 10, 11, or 12 and further comprising the amino acid sequence as set forth in SEQ ID NO:14 and a linker as set forth in SEQ ID NO: 16. In yet another embodiment, the IL-2 mutein comprises a first polypeptide comprising the amino acid sequence as set forth in SEQ ID NO:3, 4, 5, 6, 7, 8, 9, 10, 11, or 12 and further comprising the amino acid sequence as set forth in SEQ ID NO:15 and a linker as set forth in SEQ ID NO:16. In one embodiment, the IL-2 mutein comprises a first polypeptide comprising the amino acid sequence as set forth in SEQ ID NO:7 and further comprising the amino acid sequence as set forth in SEQ ID NO:13 and a linker as set forth in SEQ ID NO:16. In another embodiment, the IL-2 mutein comprises a first polypeptide comprising the amino acid sequence as set forth in SEQ ID NO:8 and further comprising the amino acid sequence as set forth in SEQ ID NO:13 and a linker as set forth in SEQ ID NO:16. In one embodiment, the IL-2 mutein comprises a first polypeptide comprising the amino acid sequence as set forth in SEQ ID NO:7 and further comprising the amino acid sequence as set forth in SEQ ID NO:14 and a linker as set forth in SEQ ID NO:16. In another embodiment, the IL-2 mutein comprises a first polypeptide comprising the amino acid sequence as set forth in SEQ ID NO:8 and further comprising the amino acid sequence as set forth in SEQ ID NO:14 and a linker as set forth in SEQ ID NO: 16. In one embodiment, the IL-2 mutein comprises a first polypeptide comprising the amino acid sequence as set forth in SEQ ID NO:7 and further comprising the amino acid sequence as set forth in SEQ ID NO:15 and a linker as set forth in SEQ ID NO:16. In another embodiment, the IL-2 mutein comprises a first polypeptide comprising the amino acid sequence as set forth in SEQ ID NO:8 and further comprising the amino acid sequence as set forth in SEQ ID NO:15 and a linker as set forth in SEQ ID NO: 16. In some embodiments, the IL-2 amino acid sequence is fused to the N-terminus of Fc or Fc variant amino acid sequence, and the peptide linker is between the C-terminus of the IL-2 and the N-terminus of the Fc or Fc variant. In some embodiments, the Fc or Fc variant amino acid sequence is fused to the N-terminus of the IL-2 amino acid sequence and the peptide linker is between the C-terminus of the Fc or Fc variant and the N-terminus of the IL-2 amino acid sequence.

In one embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO:13 and a linker as set forth in SEQ ID NO:41. In another embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO:14 and a linker as set forth in SEQ ID NO:41. In yet another embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO:15 and a linker as set forth in SEQ ID NO:41. In one embodiment, the IL-2 mutein comprises a first polypeptide comprising the amino acid sequence as set forth in SEQ ID NO:7 and further comprising the amino acid sequence as set forth in SEQ ID NO:13 and a linker as set forth in SEQ ID NO:41. In another embodiment, the IL-2 mutein comprises a first polypeptide comprising the amino acid sequence as set forth in SEQ ID NO:8 and further comprising the amino acid sequence as set forth in SEQ ID NO:13 and a linker as set forth in SEQ ID NO:41. In one embodiment, the IL-2 mutein comprises a first polypeptide comprising the amino acid sequence as set forth in SEQ ID NO:7 and further comprising the amino acid sequence as set forth in SEQ ID NO:14 and a linker as set forth in SEQ ID NO:41. In another embodiment, the IL-2 mutein comprises a first polypeptide comprising the amino acid sequence as set forth in SEQ ID NO:8 and further comprising the amino acid sequence as set forth in SEQ ID NO:14 and a linker as set forth in SEQ ID NO:41. In one embodiment, the IL-2 mutein comprises a first polypeptide comprising the amino acid sequence as set forth in SEQ ID NO:7 and further comprising the amino acid sequence as set forth in SEQ ID NO:15 and a linker as set forth in SEQ ID NO:41. In another embodiment, the IL-2 mutein comprises a first polypeptide comprising the amino acid sequence as set forth in SEQ ID NO:8 and further comprising the amino acid sequence as set forth in SEQ ID NO:15 and a linker as set forth in SEQ ID NO:41. In some embodiments, the IL-2 amino acid sequence is fused to the N-terminus of an Fc or Fc variant amino acid sequence, and the peptide linker is between the C-terminus of the IL-2 and the N-terminus of the Fc or Fc variant. In some embodiments, the Fc or Fc variant amino acid sequence is fused to the N-terminus of the IL-2 amino acid sequence and the peptide linker is between the C-terminus of the Fc or Fc variant and the N-terminus of the IL-2 amino acid sequence.

In one embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO:13 and a linker as set forth in SEQ ID NO:42. In another embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO:14 and a linker as set forth in SEQ ID NO:42. In yet another embodiment, the IL-2 mutein comprises a first polypeptide comprising an amino acid sequence as set forth in SEQ ID NO:3, 4, 5, 6, 7, 8, 9, 10, 11, or 12 and further comprising the amino acid sequence as set forth in SEQ ID NO:15 and a linker as set forth in SEQ ID NO:42. In one embodiment, the IL-2 mutein comprises a first polypeptide comprising the amino acid sequence as set forth in SEQ ID NO:7 and further comprising the amino acid sequence as set forth in SEQ ID NO:13 and a linker as set forth in SEQ ID NO:42. In another embodiment, the IL-2 mutein comprises a first polypeptide comprising the amino acid sequence as set forth in SEQ ID NO:8 and further comprising the amino acid sequence as set forth in SEQ ID NO:13 and a linker as set forth in SEQ ID NO:42. In one embodiment, the IL-2 mutein comprises a first polypeptide comprising the amino acid sequence as set forth in SEQ ID NO:7 and further comprising the amino acid sequence as set forth in SEQ ID NO:14 and a linker as set forth in SEQ ID NO:42. In another embodiment, the IL-2 mutein comprises a first polypeptide comprising the amino acid sequence as set forth in SEQ ID NO:8 and further comprising the amino acid sequence as set forth in SEQ ID NO:14 and a linker as set forth in SEQ ID NO:42. In one embodiment, the IL-2 mutein comprises a first polypeptide comprising the amino acid sequence as set forth in SEQ ID NO:7 and further comprising the amino acid sequence as set forth in SEQ ID NO:15 and a linker as set forth in SEQ ID NO:42. In another embodiment, the IL-2 mutein comprises a first polypeptide comprising the amino acid sequence as set forth in SEQ ID NO:8 and further comprising the amino acid sequence as set forth in SEQ ID NO:15 and a linker as set forth in SEQ ID NO:42. In some embodiments, the IL-2 amino acid sequence is fused to the N-terminus of an Fc or Fc variant amino acid sequence, and the peptide linker is between the C-terminus of the IL-2 and the N-terminus of the Fc or Fc variant. In some embodiments, the Fc or Fc variant amino acid sequence is fused to the N-terminus of the IL-2 amino acid sequence and the peptide linker is between the C-terminus of the Fc or Fc variant and the N-terminus of the IL-2 amino acid sequence.

In various embodiments, the IL-2 mutein further comprises a second polypeptide. In some embodiments, the second polypeptide is the same as the first polypeptide. In other embodiments, the second polypeptide is different from the first polypeptide. In yet other embodiments, the second polypeptide does not comprise a partial or full-length IL-2 amino acid sequence.

In one embodiment, the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:13 and a second polypeptide that is the same as the first polypeptide. In another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:13, and a linker sequence as set forth in SEQ ID NO:16, 41, or 42, and a second polypeptide that is the same as the first polypeptide. In yet another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:13, and a linker sequence as set forth in SEQ ID NO:16, and a second polypeptide that is the same as the first polypeptide. In still another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 13, and a linker sequence as set forth in SEQ ID NO:41, and a second polypeptide that is the same as the first polypeptide. In yet still another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:13, and a linker sequence as set forth in SEQ ID NO:42, and a second polypeptide that is the same as the first polypeptide. In one embodiment, the IL-2 mutein comprises a first polypeptide comprising the amino acid sequence as set forth in SEQ ID NO:3, the amino acid sequence as set forth in SEQ ID NO:13, and a linker sequence as set forth in SEQ ID NO:16, and a second polypeptide that is the same as the first polypeptide. In another embodiment, the IL-2 mutein comprises a first polypeptide comprising the amino acid sequence as set forth in SEQ ID NO:4, the amino acid sequence as set forth in SEQ ID NO:13, and a linker sequence as set forth in SEQ ID NO:16, and a second polypeptide that is the same as the first polypeptide. In yet another embodiment, the IL-2 mutein comprises a first polypeptide comprising the amino acid sequence as set forth in SEQ ID NO:5, the amino acid sequence as set forth in SEQ ID NO:13, and a linker sequence as set forth in SEQ ID NO:16, and a second polypeptide that is the same as the first polypeptide. In still another embodiment, the IL-2 mutein comprises a first polypeptide comprising the amino acid sequence as set forth in SEQ ID NO:6, the amino acid sequence as set forth in SEQ ID NO:13, and a linker sequence as set forth in SEQ ID NO:16, and a second polypeptide that is the same as the first polypeptide. In one embodiment, the IL-2 mutein comprises a first polypeptide comprising the amino acid sequence as set forth in SEQ ID NO:7, the amino acid sequence as set forth in SEQ ID NO:13, and a linker sequence as set forth in SEQ ID NO: 16, and a second polypeptide that is the same as the first polypeptide. In another embodiment, the IL-2 mutein comprises a first polypeptide comprising the amino acid sequence as set forth in SEQ ID NO:8, the amino acid sequence as set forth in SEQ ID NO:13, and a linker sequence as set forth in SEQ ID NO: 16, and a second polypeptide that is the same as the first polypeptide. In yet another embodiment, the IL-2 mutein comprises a first polypeptide comprising the amino acid sequence as set forth in SEQ ID NO:9, the amino acid sequence as set forth in SEQ ID NO:13, and a linker sequence as set forth in SEQ ID NO:16, and a second polypeptide that is the same as the first polypeptide. In still another embodiment, the IL-2 mutein comprises a first polypeptide comprising the amino acid sequence as set forth in SEQ ID NO:10, the amino acid sequence as set forth in SEQ ID NO:13, and a linker sequence as set forth in SEQ ID NO: 16, and a second polypeptide that is the same as the first polypeptide. In one embodiment, the IL-2 mutein comprises a first polypeptide comprising the amino acid sequence as set forth in SEQ ID NO:11, the amino acid sequence as set forth in SEQ ID NO:13, and a linker sequence as set forth in SEQ ID NO:16, and a second polypeptide that is the same as the first polypeptide. In another embodiment, the IL-2 mutein comprises a first polypeptide comprising the amino acid sequence as set forth in SEQ ID NO:12, the amino acid sequence as set forth in SEQ ID NO:13, and a linker sequence as set forth in SEQ ID NO:16, and a second polypeptide that is the same as the first polypeptide. In some embodiments, the IL-2 amino acid sequence is fused to the N-terminus of an Fc or Fc variant amino acid sequence, and the peptide linker is between the C-terminus of the IL-2 and the N-terminus of the Fc or Fc variant. In some embodiments, the Fc or Fc variant amino acid sequence is fused to the N-terminus of the IL-2 amino acid sequence and the peptide linker is between the C-terminus of the Fc or Fc variant and the N-terminus of the IL-2 amino acid sequence.

In one embodiment, the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO:17. In another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:14, and a linker sequence as set forth in SEQ ID NO:16, 41, or 42, and a second polypeptide comprising the amino acid sequence as set forth in SEQ ID NO:17. In yet another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 14, and a linker sequence as set forth in SEQ ID NO:16, and a second polypeptide comprising the amino acid sequence as set forth in SEQ ID NO: 17. In still another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:14, and a linker sequence as set forth in SEQ ID NO:41, and a second polypeptide comprising the amino acid sequence as set forth in SEQ ID NO:17. In yet still another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 14, and a linker sequence as set forth in SEQ ID NO:42, and a second polypeptide comprising the amino acid sequence as set forth in SEQ ID NO: 17. In another embodiment, the IL-2 mutein comprises a first polypeptide comprising the amino acid sequence as set forth in SEQ ID NO:3, the amino acid sequence as set forth in SEQ ID NO:14, and a linker sequence as set forth in SEQ ID NO: 16, and a second polypeptide comprising the amino acid sequence as set forth in SEQ ID NO: 17. In yet another embodiment, the IL-2 mutein comprises a first polypeptide comprising the amino acid sequence as set forth in SEQ ID NO:4, the amino acid sequence as set forth in SEQ ID NO:14, and a linker sequence as set forth in SEQ ID NO: 16, and a second polypeptide comprising the amino acid sequence as set forth in SEQ ID NO: 17. In another embodiment, the IL-2 mutein comprises a first polypeptide comprising the amino acid sequence as set forth in SEQ ID NO:5, the amino acid sequence as set forth in SEQ ID NO:14, and a linker sequence as set forth in SEQ ID NO:16, and a second polypeptide comprising the amino acid sequence as set forth in SEQ ID NO:17. In yet another embodiment, the IL-2 mutein comprises a first polypeptide comprising the amino acid sequence as set forth in SEQ ID NO:6, the amino acid sequence as set forth in SEQ ID NO:14, and a linker sequence as set forth in SEQ ID NO:16, and a second polypeptide comprising the amino acid sequence as set forth in SEQ ID NO:17. In another embodiment, the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO:16, and a second polypeptide comprising the amino acid sequence as set forth in SEQ ID NO: 17. In yet another embodiment, the IL-2 mutein comprises a first polypeptide comprising the amino acid sequence as set forth in SEQ ID NO:8, the amino acid sequence as set forth in SEQ ID NO:14, and a linker sequence as set forth in SEQ ID NO: 16, and a second polypeptide comprising the amino acid sequence as set forth in SEQ ID NO: 17. In another embodiment, the IL-2 mutein comprises a first polypeptide comprising the amino acid sequence as set forth in SEQ ID NO:9, the amino acid sequence as set forth in SEQ ID NO:14, and a linker sequence as set forth in SEQ ID NO: 16, and a second polypeptide comprising the amino acid sequence as set forth in SEQ ID NO: 17. In yet another embodiment, the IL-2 mutein comprises a first polypeptide comprising the amino acid sequence as set forth in SEQ ID NO:10, the amino acid sequence as set forth in SEQ ID NO:14, and a linker sequence as set forth in SEQ ID NO: 16, and a second polypeptide comprising the amino acid sequence as set forth in SEQ ID NO:17. In another embodiment, the IL-2 mutein comprises a first polypeptide comprising the amino acid sequence as set forth in SEQ ID NO:11, the amino acid sequence as set forth in SEQ ID NO:14, and a linker sequence as set forth in SEQ ID NO:16, and a second polypeptide comprising the amino acid sequence as set forth in SEQ ID NO:17. In yet another embodiment, the IL-2 mutein comprises a first polypeptide comprising the amino acid sequence as set forth in SEQ ID NO: 12, the amino acid sequence as set forth in SEQ ID NO:14, and a linker sequence as set forth in SEQ ID NO:16, and a second polypeptide comprising the amino acid sequence as set forth in SEQ ID NO:17. In one embodiment of the first polypeptide, the IL-2 amino acid sequence is fused to the N-terminus of an Fc or Fc variant amino acid sequence, and the peptide linker is between the C-terminus of the IL-2 and the N-terminus of the Fc or Fc variant. In another embodiment of the first polypeptide, the Fc or Fc variant amino acid sequence is fused to the N-terminus of the IL-2 amino acid sequence and the peptide linker is between the C-terminus of the Fc or Fc variant and the N-terminus of the IL-2 amino acid sequence.

In one embodiment, the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO:55. In another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:14, and a linker sequence as set forth in SEQ ID NO:16, 41, or 42, and a second polypeptide comprising the amino acid sequence as set forth in SEQ ID NO:55. In yet another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:14, and a linker sequence as set forth in SEQ ID NO:16, and a second polypeptide comprising the amino acid sequence as set forth in SEQ ID NO:55. In still another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:14, and a linker sequence as set forth in SEQ ID NO:41, and a second polypeptide comprising the amino acid sequence as set forth in SEQ ID NO:55. In yet still another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:14, and a linker sequence as set forth in SEQ ID NO:42, and a second polypeptide comprising the amino acid sequence as set forth in SEQ ID NO: 55. In another embodiment, the IL-2 mutein comprises a first polypeptide comprising the amino acid sequence as set forth in SEQ ID NO:3, the amino acid sequence as set forth in SEQ ID NO:14, and a linker sequence as set forth in SEQ ID NO: 16, and a second polypeptide comprising the amino acid sequence as set forth in SEQ ID NO:55. In yet another embodiment, the IL-2 mutein comprises a first polypeptide comprising the amino acid sequence as set forth in SEQ ID NO:4, the amino acid sequence as set forth in SEQ ID NO:14, and a linker sequence as set forth in SEQ ID NO:16, and a second polypeptide comprising the amino acid sequence as set forth in SEQ ID NO: 55. In another embodiment, the IL-2 mutein comprises a first polypeptide comprising the amino acid sequence as set forth in SEQ ID NO:5, the amino acid sequence as set forth in SEQ ID NO:14, and a linker sequence as set forth in SEQ ID NO:16, and a second polypeptide comprising the amino acid sequence as set forth in SEQ ID NO:55. In yet another embodiment, the IL-2 mutein comprises a first polypeptide comprising the amino acid sequence as set forth in SEQ ID NO:6, the amino acid sequence as set forth in SEQ ID NO: 14, and a linker sequence as set forth in SEQ ID NO:16, and a second polypeptide comprising the amino acid sequence as set forth in SEQ ID NO:55. In another embodiment, the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO: 16, and a second polypeptide comprising the amino acid sequence as set forth in SEQ ID NO:55. In yet another embodiment, the IL-2 mutein comprises a first polypeptide comprising the amino acid sequence as set forth in SEQ ID NO:8, the amino acid sequence as set forth in SEQ ID NO:14, and a linker sequence as set forth in SEQ ID NO: 16, and a second polypeptide comprising the amino acid sequence as set forth in SEQ ID NO: 55. In another embodiment, the IL-2 mutein comprises a first polypeptide comprising the amino acid sequence as set forth in SEQ ID NO:9, the amino acid sequence as set forth in SEQ ID NO:14, and a linker sequence as set forth in SEQ ID NO: 16, and a second polypeptide comprising the amino acid sequence as set forth in SEQ ID NO:55. In yet another embodiment, the IL-2 mutein comprises a first polypeptide comprising the amino acid sequence as set forth in SEQ ID NO:10, the amino acid sequence as set forth in SEQ ID NO:14, and a linker sequence as set forth in SEQ ID NO: 16, and a second polypeptide comprising the amino acid sequence as set forth in SEQ ID NO:55. In another embodiment, the IL-2 mutein comprises a first polypeptide comprising the amino acid sequence as set forth in SEQ ID NO:11, the amino acid sequence as set forth in SEQ ID NO:14, and a linker sequence as set forth in SEQ ID NO:16, and a second polypeptide comprising the amino acid sequence as set forth in SEQ ID NO:55. In yet another embodiment, the IL-2 mutein comprises a first polypeptide comprising the amino acid sequence as set forth in SEQ ID NO: 12, the amino acid sequence as set forth in SEQ ID NO:14, and a linker sequence as set forth in SEQ ID NO:16, and a second polypeptide comprising the amino acid sequence as set forth in SEQ ID NO:55. In one embodiment of the first polypeptide, the IL-2 amino acid sequence is fused to the N-terminus of an Fc or Fc variant amino acid sequence, and the peptide linker is between the C-terminus of the IL-2 and the N-terminus of the Fc or Fc variant. In another embodiment of the first polypeptide, the Fc or Fc variant amino acid sequence is fused to the N-terminus of the IL-2 amino acid sequence and the peptide linker is between the C-terminus of the Fc or Fc variant and the N-terminus of the IL-2 amino acid sequence.

In one embodiment, the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO: 15 and a second polypeptide comprising the amino acid sequence as set forth in SEQ ID NO: 18. In another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:15, and a linker sequence as set forth in SEQ ID NO:16, 41, or 42, and a second polypeptide comprising the amino acid sequence as set forth in SEQ ID NO:18. In yet another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:15, and a linker sequence as set forth in SEQ ID NO: 16, and a second polypeptide comprising the amino acid sequence as set forth in SEQ ID NO: 18. In still another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 15, and a linker sequence as set forth in SEQ ID NO:41, and a second polypeptide comprising the amino acid sequence as set forth in SEQ ID NO:18. In yet still another embodiment, the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 15, and a linker sequence as set forth in SEQ ID NO:42, and a second polypeptide comprising the amino acid sequence as set forth in SEQ ID NO: 18. In another embodiment, the IL-2 mutein comprises a first polypeptide comprising the amino acid sequence as set forth in SEQ ID NO:3, the amino acid sequence as set forth in SEQ ID NO:15, and a linker sequence as set forth in SEQ ID NO: 16, and a second polypeptide comprising the amino acid sequence as set forth in SEQ ID NO:18. In yet another embodiment, the IL-2 mutein comprises a first polypeptide comprising the amino acid sequence as set forth in SEQ ID NO:4, the amino acid sequence as set forth in SEQ ID NO:15, and a linker sequence as set forth in SEQ ID NO:16, and a second polypeptide comprising the amino acid sequence as set forth in SEQ ID NO: 18. In another embodiment, the IL-2 mutein comprises a first polypeptide comprising the amino acid sequence as set forth in SEQ ID NO:5, the amino acid sequence as set forth in SEQ ID NO:15, and a linker sequence as set forth in SEQ ID NO:16, and a second polypeptide comprising the amino acid sequence as set forth in SEQ ID NO: 18. In yet another embodiment, the IL-2 mutein comprises a first polypeptide comprising the amino acid sequence as set forth in SEQ ID NO:6, the amino acid sequence as set forth in SEQ ID NO:15, and a linker sequence as set forth in SEQ ID NO:16, and a second polypeptide comprising the amino acid sequence as set forth in SEQ ID NO:18. In another embodiment, the IL-2 mutein comprises a first polypeptide comprising the amino acid sequence as set forth in SEQ ID NO:7, the amino acid sequence as set forth in SEQ ID NO:15, and a linker sequence as set forth in SEQ ID NO:16, and a second polypeptide comprising the amino acid sequence as set forth in SEQ ID NO:18. In yet another embodiment, the IL-2 mutein comprises a first polypeptide comprising the amino acid sequence as set forth in SEQ ID NO:8, the amino acid sequence as set forth in SEQ ID NO:15, and a linker sequence as set forth in SEQ ID NO: 16, and a second polypeptide comprising the amino acid sequence as set forth in SEQ ID NO:18. In another embodiment, the IL-2 mutein comprises a first polypeptide comprising the amino acid sequence as set forth in SEQ ID NO:9, the amino acid sequence as set forth in SEQ ID NO:15, and a linker sequence as set forth in SEQ ID NO: 16, and a second polypeptide comprising the amino acid sequence as set forth in SEQ ID NO:18. In yet another embodiment, the IL-2 mutein comprises a first polypeptide comprising the amino acid sequence as set forth in SEQ ID NO:10, the amino acid sequence as set forth in SEQ ID NO:15, and a linker sequence as set forth in SEQ ID NO: 16, and a second polypeptide comprising the amino acid sequence as set forth in SEQ ID NO:18. In another embodiment, the IL-2 mutein comprises a first polypeptide comprising the amino acid sequence as set forth in SEQ ID NO:11, the amino acid sequence as set forth in SEQ ID NO:15, and a linker sequence as set forth in SEQ ID NO: 16, and a second polypeptide comprising the amino acid sequence as set forth in SEQ ID NO:18. In yet another embodiment, the IL-2 mutein comprises a first polypeptide comprising the amino acid sequence as set forth in SEQ ID NO: 12, the amino acid sequence as set forth in SEQ ID NO:15, and a linker sequence as set forth in SEQ ID NO:16, and a second polypeptide comprising the amino acid sequence as set forth in SEQ ID NO:18. In one embodiment of the first polypeptide, the IL-2 amino acid sequence is fused to the N-terminus of an Fc or Fc variant amino acid sequence, and the peptide linker is between the C-terminus of the IL-2 and the N-terminus of the Fc or Fc variant. In another embodiment of the first polypeptide, the Fc or Fc variant amino acid sequence is fused to the N-terminus of the IL-2 amino acid sequence and the peptide linker is between the C-terminus of the Fc or Fc variant and the N-terminus of the IL-2 amino acid sequence. In some embodiments, the IL-2 mutein comprises a first polypeptide and a second polypeptide, wherein the second polypeptide is the same as the first polypeptide, wherein each of the first and second polypeptides comprises an amino acid sequence as set forth in SEQ ID NO: 45, 46, 47, 48, 49, 50, 51, 52, 53, or 54. In one embodiment, each of the first and second polypeptides comprises the amino acid sequence as set forth in SEQ ID NO: 45. In one embodiment, each of the first and second polypeptides comprises the amino acid sequence as set forth in SEQ ID NO: 46. In one embodiment, each of the first and second polypeptides comprises the amino acid sequence as set forth in SEQ ID NO: 47. In one embodiment, each of the first and second polypeptides comprises the amino acid sequence as set forth in SEQ ID NO: 48. In one embodiment, each of the first and second polypeptides comprises the amino acid sequence as set forth in SEQ ID NO: 49. In one embodiment, each of the first and second polypeptides comprises the amino acid sequence as set forth in SEQ ID NO: 50. In one embodiment, each of the first and second polypeptides comprises the amino acid sequence as set forth in SEQ ID NO: 51. In one embodiment, each of the first and second polypeptides comprises the amino acid sequence as set forth in SEQ ID NO: 52. In one embodiment, each of the first and second polypeptides comprises the amino acid sequence as set forth in SEQ ID NO: 53. In one embodiment, each of the first and second polypeptides comprises the amino acid sequence as set forth in SEQ ID NO: 54.

In some embodiments, the IL-2 mutein comprises a first polypeptide and a second polypeptide, wherein the first polypeptide comprises an amino acid sequence as set forth in SEQ ID NO: 19, 20, 21, 22, 23, 24, 25, 26, 27, or 28; and the second polypeptide comprises the amino acid sequence as set forth in SEQ ID NO:17. In one embodiment, the first polypeptide comprises the amino acid sequence as set forth in SEQ ID NO:19; and the second polypeptide comprises the amino acid sequence as set forth in SEQ ID NO: 17. In another embodiment, the first polypeptide comprises the amino acid sequence as set forth in SEQ ID NO:20; and the second polypeptide comprises the amino acid sequence as set forth in SEQ ID NO:17. In yet another embodiment, the first polypeptide comprises the amino acid sequence as set forth in SEQ ID NO:21; and the second polypeptide comprises the amino acid sequence as set forth in SEQ ID NO:17. In still another embodiment, the first polypeptide comprises the amino acid sequence as set forth in SEQ ID NO:22; and the second polypeptide comprises the amino acid sequence as set forth in SEQ ID NO:17. In yet still another embodiment, the first polypeptide comprises the amino acid sequence as set forth in SEQ ID NO:23; and the second polypeptide comprises the amino acid sequence as set forth in SEQ ID NO:17. In one embodiment, the first polypeptide comprises the amino acid sequence as set forth in SEQ ID NO:24; and the second polypeptide comprises the amino acid sequence as set forth in SEQ ID NO:17. In another embodiment, the first polypeptide comprises the amino acid sequence as set forth in SEQ ID NO:25; and the second polypeptide comprises the amino acid sequence as set forth in SEQ ID NO:17. In yet another embodiment, the first polypeptide comprises an amino acid sequence as set forth in SEQ ID NO:26; and the second polypeptide comprises the amino acid sequence as set forth in SEQ ID NO:17. In still another embodiment, the first polypeptide comprises the amino acid sequence as set forth in SEQ ID NO:27; and the second polypeptide comprises the amino acid sequence as set forth in SEQ ID NO:17. In yet still another embodiment, the first polypeptide comprises the amino acid sequence as set forth in SEQ ID NO:28; and the second polypeptide comprises the amino acid sequence as set forth in SEQ ID NO: 17.

In some embodiments, the IL-2 mutein comprises a first polypeptide and a second polypeptide, wherein the first polypeptide comprises an amino acid sequence as set forth in SEQ ID NO: 19, 20, 21, 22, 23, 24, 25, 26, 27, or 28; and the second polypeptide comprises the amino acid sequence as set forth in SEQ ID NO:55. In one embodiment, the first polypeptide comprises the amino acid sequence as set forth in SEQ ID NO:19; and the second polypeptide comprises the amino acid sequence as set forth in SEQ ID NO:55. In another embodiment, the first polypeptide comprises the amino acid sequence as set forth in SEQ ID NO:20; and the second polypeptide comprises the amino acid sequence as set forth in SEQ ID NO:55. In yet another embodiment, the first polypeptide comprises the amino acid sequence as set forth in SEQ ID NO:21; and the second polypeptide comprises the amino acid sequence as set forth in SEQ ID NO:55. In still another embodiment, the first polypeptide comprises the amino acid sequence as set forth in SEQ ID NO:22; and the second polypeptide comprises the amino acid sequence as set forth in SEQ ID NO:55. In yet still another embodiment, the first polypeptide comprises the amino acid sequence as set forth in SEQ ID NO:23; and the second polypeptide comprises the amino acid sequence as set forth in SEQ ID NO:55. In one embodiment, the first polypeptide comprises the amino acid sequence as set forth in SEQ ID NO:24; and the second polypeptide comprises the amino acid sequence as set forth in SEQ ID NO:55. In another embodiment, the first polypeptide comprises the amino acid sequence as set forth in SEQ ID NO:25; and the second polypeptide comprises the amino acid sequence as set forth in SEQ ID NO:55. In yet another embodiment, the first polypeptide comprises an amino acid sequence as set forth in SEQ ID NO:26; and the second polypeptide comprises the amino acid sequence as set forth in SEQ ID NO:55. In still another embodiment, the first polypeptide comprises the amino acid sequence as set forth in SEQ ID NO:27; and the second polypeptide comprises the amino acid sequence as set forth in SEQ ID NO:55. In yet still another embodiment, the first polypeptide comprises the amino acid sequence as set forth in SEQ ID NO:28; and the second polypeptide comprises the amino acid sequence as set forth in SEQ ID NO:55.

In certain embodiments, the IL-2 mutein comprises a first polypeptide and a second polypeptide, wherein the first polypeptide comprises an amino acid sequence as set forth in SEQ ID NO:29, 30, 31, 32, 33, 34, 35, 36, 37, or 38; and the second polypeptide comprises the amino acid sequence as set forth in SEQ ID NO:18. In one embodiment, the first polypeptide comprises the amino acid sequence as set forth in SEQ ID NO:29; and the second polypeptide comprises the amino acid sequence as set forth in SEQ ID NO:18. In another embodiment, the first polypeptide comprises the amino acid sequence as set forth in SEQ ID NO:30; and the second polypeptide comprises the amino acid sequence as set forth in SEQ ID NO:18. In yet another embodiment, the first polypeptide comprises an amino acid sequence as set forth in SEQ ID NO:31; and the second polypeptide comprises the amino acid sequence as set forth in SEQ ID NO:18. In still another embodiment, the first polypeptide comprises the amino acid sequence as set forth in SEQ ID NO:32; and the second polypeptide comprises the amino acid sequence as set forth in SEQ ID NO:18. In yet still another embodiment, the first polypeptide comprises the amino acid sequence as set forth in SEQ ID NO:33; and the second polypeptide comprises the amino acid sequence as set forth in SEQ ID NO: 18. In one embodiment, the first polypeptide comprises the amino acid sequence as set forth in SEQ ID NO:34; and the second polypeptide comprises the amino acid sequence as set forth in SEQ ID NO:18. In another embodiment, the first polypeptide comprises the amino acid sequence as set forth in SEQ ID NO:35; and the second polypeptide comprises the amino acid sequence as set forth in SEQ ID NO: 18. In yet another embodiment, the first polypeptide comprises the amino acid sequence as set forth in SEQ ID NO:36; and the second polypeptide comprises the amino acid sequence as set forth in SEQ ID NO:18. In still another embodiment, the first polypeptide comprises the amino acid sequence as set forth in SEQ ID NO:37; and the second polypeptide comprises the amino acid sequence as set forth in SEQ ID NO:18. In yet still another embodiment, the first polypeptide comprises the amino acid sequence as set forth in SEQ ID NO:38; and the second polypeptide comprises the amino acid sequence as set forth in SEQ ID NO: 18.

### Methods of Using IL-2 Muteins

The invention also includes methods of using various IL-2 muteins described herein, including but not limited to preventing or treating an IL-2-mediated disease (e.g., an immune disease), selectively activating T regulatory cells without activating CD8+ T cells, or selectively activating cells that express IL-2 receptor β subunit but not activating cells that express IL-2 receptor α subunit.

Provided herein are methods of treating an IL-2-mediated disease in a subject. In certain embodiments, the method comprises administering an IL-2 mutein provided herein to the subject in a therapeutically effective amount for treating the IL-2-mediated disease. Also provided herein are methods of preventing an IL-2-mediated disease in a subject. In certain embodiments, the method comprises administering an IL-2 mutein provided herein to the subject in an amount effective for preventing the IL-2-mediated disease. In some embodiments, the subject has an IL-2-mediated disease. In other embodiments, the subject is at risk of having an IL-2-mediated disease.

In some embodiments of the various methods provided herein, the IL-2-mediated disease is an immune disorder.

In certain embodiments, the immune disorder is an inflammatory disease. In one embodiment, the inflammatory disease is uveitis.

In specific embodiments of the various methods provided herein, the immune disorder is an autoimmune disease, such as rheumatoid arthritis, Crohn's disease, psoriasis, psoriatic arthritis, multiple sclerosis, lupus, ankylosing spondylitis, type I diabetes, Sjogren's syndrome, ulcerative colitis, neuromyelitis optica, celiac disease, scleroderma, and temporal arteritis. In some embodiments, the lupus is SLE, CLE, or lupus nephritis. In one embodiment, the autoimmune disease is rheumatoid arthritis. In another embodiment, the autoimmune disease is Crohn's disease. In yet another embodiment, the autoimmune disease is psoriasis. In still another embodiment, the autoimmune disease is multiple sclerosis. In another embodiment, the autoimmune disease is ankylosing spondylitis. In yet another embodiment, the autoimmune disease is type I diabetes. In still another embodiment, the autoimmune disease is Sjogren's syndrome. In a further embodiment, the autoimmune disease is ulcerative colitis. In another embodiment, the autoimmune disease is neuromyelitis optica. In yet another embodiment, the autoimmune disease is celiac disease. In one embodiment, the autoimmune disease is temporal arteritis. In another embodiment, the autoimmune disease is scleroderma. In one embodiment, the autoimmune disease is lupus. In one embodiment, the autoimmune disease is SLE. In another embodiment, the autoimmune disease is CLE. In still another embodiment, the autoimmune disease is lupus nephritis.

In other embodiments of the various methods provided herein, the immune disorder is a hypersensitivity disease, including, for example, atopic dermatitis, hypersensitivity vasculitis, and allergy, e.g., allergic asthma, allergic rhinitis (hay fever), urticaria (hives), and anaphylaxis. In one embodiment, the immune disorder is atopic dermatitis. In another embodiment, the immune disorder is hypersensitivity vasculitis. In yet another embodiment, the immune disorder is allergy. In one embodiment, the immune disorder is allergic asthma. In another embodiment, the immune disorder is allergic rhinitis (hay fever). In yet another embodiment, the immune disorder is urticaria (hives). In certain embodiments, the immune disorder is anaphylaxis.

In certain embodiments, the hypersensitivity disease is a T cell hypersensitivity disease. The term “T cell hypersensitivity,” when used in reference to an immune disorder of a subject, refers to a transient or chronic abnormally high level of T cell effector function. In certain embodiments, the T cell effector function comprises secretion of TH2 cytokines. Exemplary TH2 cytokines include but are not limited to IL-2, IL-4, IL-9, IL-13, IL-31, and TSLP. In one embodiment, the TH2 cytokine is IL-2. In some embodiments, the subject has a transient or chronic abnormally high level of two, three, four, five, six, seven, eight, nine, or ten different TH2 cytokines, one of which is IL-2.

In some embodiments, the immune disease is alopecia areata, graft versus host disease (GVHD), autoimmune hepatitis, primary sclerosing cholangitis, or inflammatory myopathy. In one embodiment, the immune disease is alopecia areata. In another embodiment, the immune disease is GVHD. In yet another embodiment, the immune disease is autoimmune hepatitis. In still another embodiment, the immune disease is primary sclerosing cholangitis. In yet still another embodiment, the immune disease is inflammatory myopathy.

Thus, in some embodiments, provided is a method of treating an IL-2-mediated disease in a subject, comprising administering to the subject a therapeutically effective amount of any one of the various IL-2 muteins described herein or any one of the various pharmaceutical compositions described herein.

In certain embodiments, provided is a method of treating an immune disorder in a subject, comprising administering to the subject a therapeutically effective amount of any one of the various IL-2 muteins described herein or any one of the various pharmaceutical compositions described herein.

In other embodiments, provided is a method of treating an autoimmune disease in a subject, comprising administering to the subject a therapeutically effective amount of any one of the various IL-2 muteins described herein or any one of the various pharmaceutical compositions described herein.

In yet other embodiments, provided is a method of treating an inflammatory disease in a subject, comprising administering to the subject a therapeutically effective amount of any one of the various IL-2 muteins described herein or any one of the various pharmaceutical compositions described herein.

In a specific embodiment, provided is a method of treating GVHD in a subject, comprising administering to the subject a therapeutically effective amount of any one of the various IL-2 muteins described herein or any one of the various pharmaceutical compositions described herein.

In another specific embodiment, provided is a method of treating SLE in a subject, comprising administering to the subject a therapeutically effective amount of any one of the various IL-2 muteins described herein or any one of the various pharmaceutical compositions described herein.

In yet another specific embodiment, provided is a method of treating CLE in a subject, comprising administering to the subject a therapeutically effective amount of any one of the various IL-2 muteins described herein or any one of the various pharmaceutical compositions described herein.

In still another specific embodiment, provided is a method of treating multiple sclerosis in a subject, comprising administering to the subject a therapeutically effective amount of any one of the various IL-2 muteins described herein or any one of the various pharmaceutical compositions described herein.

In yet still another specific embodiment, provided is a method of treating ulcerative colitis in a subject, comprising administering to the subject a therapeutically effective amount of any one of the various IL-2 muteins described herein or any one of the various pharmaceutical compositions described herein.

In still another specific embodiment, provided is a method of treating Crohn's disease in a subject, comprising administering to the subject a therapeutically effective amount of any one of the various IL-2 muteins described herein or any one of the various pharmaceutical compositions described herein.

In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO: 17. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 14, and a linker sequence as set forth in SEQ ID NO: 16, and a second polypeptide comprising the amino acid sequence as set forth in SEQ ID NO: 17. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO:16, and a second polypeptide comprising the amino acid sequence as set forth in SEQ ID NO:17. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:14, and a linker sequence as set forth in SEQ ID NO: 16, and a second polypeptide comprising the amino acid sequence as set forth in SEQ ID NO: 17. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO: 19, 20, 21, 22, 23, 24, 25, 26, 27, or 28, and a second polypeptide comprising the amino acid sequence as set forth in SEQ ID NO:17. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO:17. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO:17.

In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO:55. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 14, and a linker sequence as set forth in SEQ ID NO:16, and a second polypeptide comprising the amino acid sequence as set forth in SEQ ID NO:55. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO:16, and a second polypeptide comprising the amino acid sequence as set forth in SEQ ID NO:55. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:14, and a linker sequence as set forth in SEQ ID NO: 16, and a second polypeptide comprising the amino acid sequence as set forth in SEQ ID NO:55. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:19, 20, 21, 22, 23, 24, 25, 26, 27, or 28, and a second polypeptide comprising the amino acid sequence as set forth in SEQ ID NO:55. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO:55. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO:55.

In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:15 and a second polypeptide comprising the amino acid sequence as set forth in SEQ ID NO: 18. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 15, and a linker sequence as set forth in SEQ ID NO: 16, and a second polypeptide comprising the amino acid sequence as set forth in SEQ ID NO: 18. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:15, and a linker sequence as set forth in SEQ ID NO:16, and a second polypeptide comprising the amino acid sequence as set forth in SEQ ID NO:18. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:15, and a linker sequence as set forth in SEQ ID NO:16, and a second polypeptide comprising the amino acid sequence as set forth in SEQ ID NO:18. In a specific embodiment, provided is a method of treating GVHD in a subject, comprising administering to the subject a therapeutically effective amount of the IL-2 mutein or pharmaceutical composition thereof, wherein the IL-2 mutein comprises a first polypeptide comprising an amino acid sequence as set forth in SEQ ID NO:29, 30, 31, 32, 33, 34, 35, 36, 37, or 38, and a second polypeptide comprising the amino acid sequence as set forth in SEQ ID NO:18. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:33, and a second polypeptide comprising the amino acid sequence as set forth in SEQ ID NO: 18. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:34, and a second polypeptide comprising the amino acid sequence as set forth in SEQ ID NO:18.

In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:13 and a second polypeptide that is the same as the first polypeptide. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:13, and a linker sequence as set forth in SEQ ID NO:16, and a second polypeptide comprising the amino acid sequence that is the same amino acid sequence as the first polypeptide. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:13, and a linker sequence as set forth in SEQ ID NO:16, and a second polypeptide comprising amino acid sequence that is the same amino acid sequence as the first polypeptide. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:13, and a linker sequence as set forth in SEQ ID NO:16, and a second polypeptide comprising an amino acid sequence that is the same amino acid sequence as the first polypeptide. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:45, 46, 47, 48, 49, 50, 51, 52, 53, 54, and a second polypeptide comprising an amino acid sequence that is the same amino acid sequence as the first polypeptide. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:45, and a second polypeptide comprising the amino acid sequence as set forth in SEQ ID NO:45. In a specific embodiment, provided is a method of treating GVHD in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:46, and a second polypeptide comprising the amino acid sequence as set forth in SEQ ID NO:46.

In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO: 17. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 14, and a linker sequence as set forth in SEQ ID NO:16, and a second polypeptide comprising an amino acid sequence as set forth in SEQ ID NO:17. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO:16, and a second polypeptide comprising the amino acid sequence as set forth in SEQ ID NO:17. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the an amino acid sequence as set forth in SEQ ID NO:8, the amino acid sequence as set forth in SEQ ID NO:14, and a linker sequence as set forth in SEQ ID NO:16, and a second polypeptide comprising the amino acid sequence as set forth in SEQ ID NO: 17. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:19, 20, 21, 22, 23, 24, 25, 26, 27, or 28, and a second polypeptide comprising the amino acid sequence as set forth in SEQ ID NO:17. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO: 17. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO:17.

In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO:55. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:14, and a linker sequence as set forth in SEQ ID NO:16, and a second polypeptide comprising an amino acid sequence as set forth in SEQ ID NO:55. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO:16, and a second polypeptide comprising the amino acid sequence as set forth in SEQ ID NO:55. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the an amino acid sequence as set forth in SEQ ID NO:8, the amino acid sequence as set forth in SEQ ID NO:14, and a linker sequence as set forth in SEQ ID NO:16, and a second polypeptide comprising the amino acid sequence as set forth in SEQ ID NO:55. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:19, 20, 21, 22, 23, 24, 25, 26, 27, or 28, and a second polypeptide comprising the amino acid sequence as set forth in SEQ ID NO:55. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO:55. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO:55.

In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:15 and a second polypeptide comprising the amino acid sequence as set forth in SEQ ID NO:18. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 15, and a linker sequence as set forth in SEQ ID NO: 16, and a second polypeptide comprising the amino acid sequence as set forth in SEQ ID NO: 18. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:15, and a linker sequence as set forth in SEQ ID NO:16, and a second polypeptide comprising the amino acid sequence as set forth in SEQ ID NO:18. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:15, and a linker sequence as set forth in SEQ ID NO: 16, and a second polypeptide comprising the amino acid sequence as set forth in SEQ ID NO: 18. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:29, 30, 31, 32, 33, 34, 35, 36, 37, or 38, and a second polypeptide comprising the amino acid sequence as set forth in SEQ ID NO:18. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:33, and a second polypeptide comprising the amino acid sequence as set forth in SEQ ID NO: 18. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:34, and a second polypeptide comprising the amino acid sequence as set forth in SEQ ID NO:18.

In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:13 and a second polypeptide that is the same as the first polypeptide. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:13, and a linker sequence as set forth in SEQ ID NO:16, and a second polypeptide comprising an amino acid sequence that is the same amino acid sequence as the first polypeptide. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:13, and a linker sequence as set forth in SEQ ID NO:16, and a second polypeptide comprising an amino acid sequence that is the same amino acid sequence as the first polypeptide. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:13, and a linker sequence as set forth in SEQ ID NO:16, and a second polypeptide comprising an amino acid sequence that is the same amino acid sequence as the first polypeptide. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:45, 46, 47, 48, 49, 50, 51, 52, 53, 54, and a second polypeptide comprising amino acid sequence that is the same amino acid sequence as the first polypeptide. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:45, and a second polypeptide comprising the amino acid sequence as set forth in SEQ ID NO:45. In a specific embodiment, provided is a method of treating SLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:46, and a second polypeptide comprising the amino acid sequence as set forth in SEQ ID NO: 46.

In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO: 17. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 14, and a linker sequence as set forth in SEQ ID NO:16, and a second polypeptide comprising the amino acid sequence as set forth in SEQ ID NO:17. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO: 16, and a second polypeptide comprising the amino acid sequence as set forth in SEQ ID NO:17. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:14, and a linker sequence as set forth in SEQ ID NO: 16, and a second polypeptide comprising the amino acid sequence as set forth in SEQ ID NO:17. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:19, 20, 21, 22, 23, 24, 25, 26, 27, or 28, and a second polypeptide comprising an amino acid sequence as set forth in SEQ ID NO:17. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO: 17. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO:17.

In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO:55. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 14, and a linker sequence as set forth in SEQ ID NO:16, and a second polypeptide comprising the amino acid sequence as set forth in SEQ ID NO:55. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO: 16, and a second polypeptide comprising the amino acid sequence as set forth in SEQ ID NO:55. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:14, and a linker sequence as set forth in SEQ ID NO: 16, and a second polypeptide comprising the amino acid sequence as set forth in SEQ ID NO:55. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:19, 20, 21, 22, 23, 24, 25, 26, 27, or 28, and a second polypeptide comprising an amino acid sequence as set forth in SEQ ID NO:55. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO:55. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO:55.

In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO: 15 and a second polypeptide comprising the amino acid sequence as set forth in SEQ ID NO:18. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 15, and a linker sequence as set forth in SEQ ID NO: 16, and a second polypeptide comprising an amino acid sequence as set forth in SEQ ID NO: 18. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:15, and a linker sequence as set forth in SEQ ID NO: 16, and a second polypeptide comprising an amino acid sequence as set forth in SEQ ID NO:18. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:15, and a linker sequence as set forth in SEQ ID NO: 16, and a second polypeptide comprising the amino acid sequence as set forth in SEQ ID NO: 18. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:29, 30, 31, 32, 33, 34, 35, 36, 37, or 38, and a second polypeptide comprising the amino acid sequence as set forth in SEQ ID NO:18. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:33, and a second polypeptide comprising the amino acid sequence as set forth in SEQ ID NO: 18. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:34, and a second polypeptide comprising the amino acid sequence as set forth in SEQ ID NO:18.

In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and an amino acid sequence as set forth in SEQ ID NO:13 and a second polypeptide that is the same as the first polypeptide. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:13, and a linker sequence as set forth in SEQ ID NO:16, and a second polypeptide comprising acid sequence that is the same amino acid sequence as the first polypeptide. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:13, and a linker sequence as set forth in SEQ ID NO: 16, and a second polypeptide comprising an amino acid sequence that is the same amino acid sequence as the first polypeptide. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:13, and a linker sequence as set forth in SEQ ID NO:16, and a second polypeptide comprising an amino acid sequence that is the same amino acid sequence as the first polypeptide. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:45, 46, 47, 48, 49, 50, 51, 52, 53, 54, and a second polypeptide comprising an amino acid sequence that is the same amino acid sequence as the first polypeptide. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:45, and a second polypeptide comprising the amino acid sequence as set forth in SEQ ID NO:45. In a specific embodiment, provided is a method of treating CLE in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:46, and a second polypeptide comprising the amino acid sequence as set forth in SEQ ID NO:46.

In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO: 14 and a second polypeptide comprising the amino acid sequence as set forth in SEQ ID NO:17. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:14, and a linker sequence as set forth in SEQ ID NO:16, and a second polypeptide comprising the amino acid sequence as set forth in SEQ ID NO:17. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO: 16, and a second polypeptide comprising the amino acid sequence as set forth in SEQ ID NO: 17. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:14, and a linker sequence as set forth in SEQ ID NO:16, and a second polypeptide comprising the amino acid sequence as set forth in SEQ ID NO: 17. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:19, 20, 21, 22, 23, 24, 25, 26, 27, or 28, and a second polypeptide comprising the amino acid sequence as set forth in SEQ ID NO:17. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO:17. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO: 17.

In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO:55. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:14, and a linker sequence as set forth in SEQ ID NO:16, and a second polypeptide comprising the amino acid sequence as set forth in SEQ ID NO:55. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO: 16, and a second polypeptide comprising the amino acid sequence as set forth in SEQ ID NO:55. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:14, and a linker sequence as set forth in SEQ ID NO:16, and a second polypeptide comprising the amino acid sequence as set forth in SEQ ID NO:55. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:19, 20, 21, 22, 23, 24, 25, 26, 27, or 28, and a second polypeptide comprising the amino acid sequence as set forth in SEQ ID NO:55. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO:55. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO: 55.

In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:15 and a second polypeptide comprising the amino acid sequence as set forth in SEQ ID NO:18. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:15, and a linker sequence as set forth in SEQ ID NO:16, and a second polypeptide comprising an amino acid sequence as set forth in SEQ ID NO:18. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:15, and a linker sequence as set forth in SEQ ID NO: 16, and a second polypeptide comprising the amino acid sequence as set forth in SEQ ID NO: 18. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:15, and a linker sequence as set forth in SEQ ID NO:16, and a second polypeptide comprising the amino acid sequence as set forth in SEQ ID NO:18. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:29, 30, 31, 32, 33, 34, 35, 36, 37, or 38, and a second polypeptide comprising the amino acid sequence as set forth in SEQ ID NO:18. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:33, and a second polypeptide comprising the amino acid sequence as set forth in SEQ ID NO:18. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO: 34, and a second polypeptide comprising the amino acid sequence as set forth in SEQ ID NO: 18.

In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:13 and a second polypeptide that is the same as the first polypeptide. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 13, and a linker sequence as set forth in SEQ ID NO:16, and a second polypeptide comprising an amino acid sequence that is the same amino acid sequence as the first polypeptide. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:13, and a linker sequence as set forth in SEQ ID NO: 16, and a second polypeptide comprising an amino acid sequence that is the same amino acid sequence as the first polypeptide. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:13, and a linker sequence as set forth in SEQ ID NO: 16, and a second polypeptide comprising an amino acid sequence that is the same amino acid sequence as the first polypeptide. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:45, 46, 47, 48, 49, 50, 51, 52, 53, 54, and a second polypeptide comprising an amino acid sequence that is the same amino acid sequence as the first polypeptide. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:45, and a second polypeptide comprising the amino acid sequence as set forth in SEQ ID NO:45. In a specific embodiment, provided is a method of treating multiple sclerosis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:46, and a second polypeptide comprising the amino acid sequence as set forth in SEQ ID NO:46.

In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO: 14 and a second polypeptide comprising the amino acid sequence as set forth in SEQ ID NO: 17. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 14, and a linker sequence as set forth in SEQ ID NO:16, and a second polypeptide comprising the amino acid sequence as set forth in SEQ ID NO:17. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO: 16, and a second polypeptide comprising the amino acid sequence as set forth in SEQ ID NO: 17. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO: 14, and a linker sequence as set forth in SEQ ID NO: 16, and a second polypeptide comprising the amino acid sequence as set forth in SEQ ID NO:17. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:19, 20, 21, 22, 23, 24, 25, 26, 27, or 28, and a second polypeptide comprising the amino acid sequence as set forth in SEQ ID NO:17. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO:17. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO:17.

In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO:55. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 14, and a linker sequence as set forth in SEQ ID NO:16, and a second polypeptide comprising the amino acid sequence as set forth in SEQ ID NO:55. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO: 16, and a second polypeptide comprising the amino acid sequence as set forth in SEQ ID NO:55. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO: 14, and a linker sequence as set forth in SEQ ID NO:16, and a second polypeptide comprising the amino acid sequence as set forth in SEQ ID NO:55. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:19, 20, 21, 22, 23, 24, 25, 26, 27, or 28, and a second polypeptide comprising the amino acid sequence as set forth in SEQ ID NO:55. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO:55. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO: 55.

In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO: 15 and a second polypeptide comprising the amino acid sequence as set forth in SEQ ID NO:18. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 15, and a linker sequence as set forth in SEQ ID NO:16, and a second polypeptide comprising the amino acid sequence as set forth in SEQ ID NO:18. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:15, and a linker sequence as set forth in SEQ ID NO: 16, and a second polypeptide comprising the amino acid sequence as set forth in SEQ ID NO: 18. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:15, and a linker sequence as set forth in SEQ ID NO:16, and a second polypeptide comprising the amino acid sequence as set forth in SEQ ID NO:18. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:29, 30, 31, 32, 33, 34, 35, 36, 37, or 38, and a second polypeptide comprising the amino acid sequence as set forth in SEQ ID NO:18. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:33, and a second polypeptide comprising the amino acid sequence as set forth in SEQ ID NO:18. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:34, and a second polypeptide comprising the amino acid sequence as set forth in SEQ ID NO:18.

In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and an amino acid sequence as set forth in SEQ ID NO:13 and a second polypeptide that is the same as the first polypeptide. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO: 3, 4, 5, 6, 7, 8, 9, 10, 11, or 12, an amino acid sequence as set forth in SEQ ID NO: 13, and a linker sequence as set forth in SEQ ID NO:16, and a second polypeptide comprising an amino acid sequence that is the same amino acid sequence as the first polypeptide. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:13, and a linker sequence as set forth in SEQ ID NO:16, and a second polypeptide comprising an amino acid sequence that is the same amino acid sequence as the first polypeptide. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:13, and a linker sequence as set forth in SEQ ID NO: 16, and a second polypeptide comprising an amino acid sequence that is the same amino acid sequence as the first polypeptide. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:45, 46, 47, 48, 49, 50, 51, 52, 53, 54, and a second polypeptide comprising an amino acid sequence that is the same amino acid sequence as the first polypeptide. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:45, and a second polypeptide comprising the amino acid sequence as set forth in SEQ ID NO:45. In a specific embodiment, provided is a method of treating ulcerative colitis in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:46, and a second polypeptide comprising the amino acid sequence as set forth in SEQ ID NO:46

In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO:17. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:14, and a linker sequence as set forth in SEQ ID NO:16, and a second polypeptide comprising the amino acid sequence as set forth in SEQ ID NO:17. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO: 16, and a second polypeptide comprising the amino acid sequence as set forth in SEQ ID NO: 17. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:14, and a linker sequence as set forth in SEQ ID NO:16, and a second polypeptide comprising the amino acid sequence as set forth in SEQ ID NO:17. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:19, 20, 21, 22, 23, 24, 25, 26, 27, or 28, and a second polypeptide comprising the amino acid sequence as set forth in SEQ ID NO:17. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO:17. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO: 17.

In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:14 and a second polypeptide comprising the amino acid sequence as set forth in SEQ ID NO:55. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:14, and a linker sequence as set forth in SEQ ID NO:16, and a second polypeptide comprising the amino acid sequence as set forth in SEQ ID NO:55. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:14, and a linker sequence as set forth in SEQ ID NO: 16, and a second polypeptide comprising the amino acid sequence as set forth in SEQ ID NO:55. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:14, and a linker sequence as set forth in SEQ ID NO:16, and a second polypeptide comprising the amino acid sequence as set forth in SEQ ID NO:55. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:19, 20, 21, 22, 23, 24, 25, 26, 27, or 28, and a second polypeptide comprising the amino acid sequence as set forth in SEQ ID NO:55. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:23, and a second polypeptide comprising the amino acid sequence as set forth in SEQ ID NO:55. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:24, and a second polypeptide comprising the amino acid sequence as set forth in SEQ ID NO:55.

In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO: 15 and a second polypeptide comprising the amino acid sequence as set forth in SEQ ID NO:18. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO:15, and a linker sequence as set forth in SEQ ID NO:16, and a second polypeptide comprising the amino acid sequence as set forth in SEQ ID NO:18. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, an amino acid sequence as set forth in SEQ ID NO:15, and a linker sequence as set forth in SEQ ID NO: 16, and a second polypeptide comprising the amino acid sequence as set forth in SEQ ID NO: 18. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:15, and a linker sequence as set forth in SEQ ID NO:16, and a second polypeptide comprising the amino acid sequence as set forth in SEQ ID NO:18. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:29, 30, 31, 32, 33, 34, 35, 36, 37, or 38, and a second polypeptide comprising the amino acid sequence as set forth in SEQ ID NO:18. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:33, and a second polypeptide comprising the amino acid sequence as set forth in SEQ ID NO:18. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:34, and a second polypeptide comprising the amino acid sequence as set forth in SEQ ID NO:18.

In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12 and the amino acid sequence as set forth in SEQ ID NO:13 and a second polypeptide that is the same as the first polypeptide. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:3, 4, 5, 6, 7, 8, 9, 10, 11, or 12, the amino acid sequence as set forth in SEQ ID NO: 13, and a linker sequence as set forth in SEQ ID NO:16, and a second polypeptide comprising an amino acid sequence that is the same amino acid sequence as the first polypeptide. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:7, the amino acid sequence as set forth in SEQ ID NO:13, and a linker sequence as set forth in SEQ ID NO:16, and a second polypeptide comprising an amino acid sequence that is the same amino acid sequence as the first polypeptide. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:8, the amino acid sequence as set forth in SEQ ID NO:13, and a linker sequence as set forth in SEQ ID NO: 16, and a second polypeptide comprising an amino acid sequence that is the same amino acid sequence as the first polypeptide. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising an amino acid sequence as set forth in SEQ ID NO:45, 46, 47, 48, 49, 50, 51, 52, 53, 54, and a second polypeptide comprising an amino acid sequence that is the same amino acid sequence as the first polypeptide. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:45, and a second polypeptide comprising the amino acid sequence as set forth in SEQ ID NO:45. In a specific embodiment, provided is a method of treating Crohn's disease in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:46, and a second polypeptide comprising the amino acid sequence as set forth in SEQ ID NO:46.

In still another aspect, provided is a method of selectively activating T regulatory cells without activating CD8+ T cells in a subject, comprising administering to the subject in need thereof a therapeutic effective amount of the various IL-2 mutein described herein or the various pharmaceutical composition described herein. In some embodiments, selectively activating T regulatory cells without activating CD8+ T cells can be measured by in vitro or ex vivo methods. In a specific embodiment, selectively activating T regulatory cells without activating CD8+ T cells can be measured by any methods known by a person of ordinary skills in the art, including but not limited to pSTAT5 assay described in the examples provided herein.

In yet still another aspect, provided is a method of selectively activating cells that express IL-2 receptor β subunit but not activating cells that express IL-2 receptor α subunit in a subject, comprising administering to the subject in need thereof a therapeutic effective amount of the various IL-2 mutein described herein or the various pharmaceutical composition described herein. In some embodiments, selectively activating cells that express IL-2 receptor β subunit but not activating cells that express IL-2 receptor α subunit can be measured by in vitro or ex vivo methods. In a specific embodiment, selectively activating cells that express IL-2 receptor 3 subunit but not activating cells that express IL-2 receptor α subunit can be measured by any methods known by a person of ordinary skills in the art, including but not limited to pSTAT5 assay described in the examples provided herein.

In yet still another aspect, provided is use of any one of the various IL-2 muteins described herein or any one of the various pharmaceutical compositions described herein to treat an IL-2-mediated disease in a subject.

In one aspect, provided is use of any one of the various IL-2 muteins described herein or any one of the various pharmaceutical compositions described herein for the treatment of an IL-2-mediated disease in a subject.

In another aspect, provided is use of any one of the various IL-2 muteins described herein or any one of the various pharmaceutical compositions described herein for the preparation of a medicament to treat an IL-2-mediated disease in a subject.

In various uses of the IL-2 muteins described herein, the IL-2-mediated disease can be any immune disorder (e.g., an autoimmune or inflammatory disease) described in this disclosure.

### Nucleic Acids, Expression Vectors, Cells, and Methods of Making IL-2 Muteins

Also provided herein are isolated nucleic acids and vectors comprising polynucleotide sequences encoding such IL-2 muteins disclosed herein, cells (e.g., host cells) comprising such isolated nucleic acids or vectors, and methods of making such IL-2 muteins.

In one aspect, provided is an isolated nucleic acid, encoding a polypeptide of any one of the IL-2 muteins described herein. In one embodiment, the isolated nucleic acid encodes any one of the various first polypeptides of the various IL-2 muteins described herein. In another embodiment, the isolated nucleic acid encodes any one of the various second polypeptides of the various IL-2 muteins described herein. In yet another embodiment, the isolated nucleic acid encodes any one of the various first polypeptides and any one of the various second polypeptides of the various IL-2 muteins described herein. In still another embodiment, the isolated nucleic acid encodes one or more polypeptides disclosed in Table 9.

In another aspect, provided is a first isolated nucleic acid that encodes the first polypeptide and a second isolated nucleic acid that encodes the second polypeptide of an IL-2 mutein described herein.

In another embodiment, the isolated nucleic acids further encode a signal sequence.

In another aspect, provided is an expression vector comprising one or more of the various isolated nucleic acids disclosed herein, wherein the nucleic acid(s) is operably linked to control sequences that are recognized by a host cell when the host cell is transfected with the expression vector.

In yet another aspect, provided is host cell comprising one or more of the various isolated nucleic acids or the various expression vectors disclosed herein. In some embodiments, the host cell comprises one or more of the various isolated nucleic acids disclosed herein. In other embodiments, the host cell comprises one or more of the various expression vectors disclosed herein. In yet other embodiments, the host cell comprises one of the various expression vectors disclosed herein, wherein the expression vector comprises one or more of the various isolated nucleic acids disclosed herein. In still other embodiments, the host cell comprises a first expression vector comprising a first nucleic acid that encodes the first polypeptide and a second expression vector comprising a second nucleic acid that encodes the second polypeptide of the IL-2 mutein described herein.

In still another aspect, provided is a method of producing any one of the various IL-2 muteins described herein. In one embodiment, the method comprises culturing any one of the various host cells described herein under conditions wherein the IL-2 mutein is expressed. In another embodiment, the method comprises expressing any one of the various expression vectors described herein under conditions wherein the IL-2 mutein is expressed. In yet another embodiment, the method comprises expressing any one of the various isolated nucleic acids described herein under conditions wherein the IL-2 mutein is expressed.

In certain embodiments of the various methods of making the IL-2 muteins of the invention, the method further comprises isolating the IL-2 mutein from the host cell or culture medium, or in vitro expression system.

Mammalian cell lines available as hosts for expression of the IL-2 muteins disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Other cell lines that may be used are insect cell lines, such as Sf9 cells, amphibian cells, bacterial cells, plant cells and fungal cells. Various modifications can be introduced into the genome of these cell lines (e.g., glutamine synthetase knockout, auxotrophic mutations, etc.) to achieve desired properties of the host cells and/or desired properties of the expressed IL-2 muteins.

When recombinant expression vectors encoding the IL-2 mutein are introduced into host cells, the IL-2 mutein is produced by culturing the host cells for a period of time sufficient to allow for expression of the IL-2 mutein in the host cells or, more preferably, secretion of the IL-2 mutein into the culture medium in which the host cells are grown.

IL-2 muteins can be recovered from the culture medium using standard protein purification methods.

In general, glycoproteins produced in a particular cell line or transgenic animal will have a glycosylation pattern that is characteristic for glycoproteins produced in the cell line or transgenic animal. Therefore, the particular glycosylation pattern of an IL-2 mutein will depend on the particular cell line or transgenic animal used to produce the IL-2 mutein. The disclosures of various IL-2 muteins or isolated nucleic acids encoding such IL-2 muteins are independent of the glycosylation pattern the IL-2 muteins may have.

### Pharmaceutical Compositions and Administration

In another aspect, provided is a composition comprising any one of the IL-2 muteins described herein and a pharmaceutically acceptable carrier.

In some embodiments, the composition further comprises an additional agent.

In particular embodiments, the additional agent is an agent effective to treat the same disorder that the IL-2 muteins disclosed herein are being used to treat. In some embodiments, the additional agent is an agent effective to relieve side effects of the IL-2 muteins disclosed herein.

To prepare pharmaceutical or sterile compositions of the IL-2 muteins described herein, an IL-2 mutein of the invention is admixed with a pharmaceutically acceptable carrier or excipient. See, e.g., Remington's Pharmaceutical Sciences and U.S. Pharmacopeia: National Formulary, Mack Publishing Company, Easton, PA (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY).

Toxicity and therapeutic efficacy of the IL-2 mutein compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In a further embodiment, a composition comprising an IL-2 mutein disclosed herein is administered to a subject in accordance with the Physicians' Desk Reference 2003 (Thomson Healthcare; 57th edition (Nov. 1, 2002)).

The mode of administration of the IL-2 muteins and compositions of the invention can vary. Suitable routes of administration include oral, rectal, transmucosal, intestinal, parenteral, intramuscular, subcutaneous, intradermal, intramedullary, intrathecal, direct intraventricular, intravenous, intraperitoneal, intranasal, intraocular, inhalation, insufflation, topical, cutaneous, transdermal, and intra-arterial.

In particular embodiments, an IL-2 mutein of the invention can be administered by an invasive route such as by injection. In further embodiments, the IL-2 mutein, or pharmaceutical composition thereof, is administered intravenously, subcutaneously, intrathecally, intramuscularly, or intracerebrally. In one specific embodiment, the IL-2 mutein, or pharmaceutical composition thereof, is administered intravenously. In another specific embodiment, the IL-2 mutein, or pharmaceutical composition thereof, is administered subcutaneously.

Compositions can be administered with medical devices known in the art. For example, a pharmaceutical composition of the invention can be administered by injection with a hypodermic needle, including, e.g., a prefilled syringe or autoinjector.

The pharmaceutical compositions disclosed herein may also be administered by infusion.

The administration regimen depends on several factors, including the serum or tissue turnover rate of the IL-2 mutein, the level of symptoms, the immunogenicity of the IL-2 mutein, and the accessibility of the target cells in the biological matrix. Preferably, the administration regimen delivers sufficient IL-2 mutein to effect improvement in the target disease state, while simultaneously minimizing undesired side effects. Accordingly, the amount of biologic delivered depends in part on the particular IL-2 mutein and the severity of the condition being treated. Guidance in selecting appropriate doses is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J Med. 348:601-608; Milgrom et al. (1999) New Engl. J Med. 341:1966-1973; Slamon et al. (2001) New Engl. J Med. 344:783-792; Beniaminovitz et al. (2000) New Engl. J Med. 342:613-619; Ghosh et al. (2003) New Engl. J Med. 348:24-32; Lipsky et al. (2000) New Engl. J. Med. 343:1594-1602).

Determination of the appropriate dose is made by the clinician, e.g., using parameters or factors known or suspected in the art to affect treatment. In some embodiments, the dose begins with an amount somewhat less than the optimum dose and it is increased by small increments thereafter until the desired or optimum effect is achieved relative to any negative side effects.

As previously described, the IL-2 mutein may be co-administered with one or more additional agents. The IL-2 mutein can be linked to the agent (such as a conjugate) or can be administered separately from the agent. In the latter case (separate administration), the IL-2 mutein can be administered before, after, or concurrently with the agent or can be co-administered with other known therapies.

### Kits

Also provided herein are kits comprising an IL-2 mutein provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage.

### General Methods

Standard methods in molecular biology are described in Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J Biochem. 133:17-21; von Heijne (1986) Nucleic Acids Res. 14:4683-4690).

### Example 1. Cloning, Expression, and Purification of IL-2 Muteins

Polynucleotides encoding various IL-2 muteins were synthesized and cloned into the PTT5 plasmid (Canadian National Research Council). ExpiCHO or Expi293 cells growing in Gibco ExpiCHO or Expi293 Expression Medium in suspension were transiently transfected with the PTT5 constructs comprising the polynucleotides encoding the various IL-2 muteins, using commercially available reagent ExpiFectamine and protocols (Thermo-Fisher). In brief, cells were transfected day 0 using 1 μg total DNA per 1 mL cells with viability>95% measured using a Vi-Cell (Beckman-Coulter). For expressing bivalent IL-2 muteins, a PTT5 construct encoding an IL-2 mutein containing a Fc variant Fc-1 (SEQ ID NO:13) was transfected. For some embodiments of expressing monovalent IL-2 muteins, a first PTT5 construct encoding an IL-2 mutein containing a Fc variant Fc-2 (SEQ ID NO: 14) and a second PTT5 construct encoding a Fc variant Fc-4 (SEQ ID NO:17) were co-transfected, and the ratio of the first PTT5 construct to the second PTT5 construct was 3:1. In other embodiments of expressing monovalent IL-2 muteins, a first PTT5 construct encoding an IL-2 mutein containing a Fc variant Fc-3 (SEQ ID NO:15) and a second PTT5 construct encoding a Fc variant Fc-5 (SEQ ID NO:18) are co-transfected, and the ratio of the first PTT5 construct to the second PTT5 construct is 3:1. Cultures were harvested on day 5 for Expi293 cells and between day 8 and day 12 depending on a cell viability greater than 80% for ExpiCHO cells. Monovalent or bivalent IL-2 muteins were purified from clarified supernatant using Protein A chromatography (mAbSelect Sure LX, GE Healthcare) on AKTA FPLC systems (GE Healthcare). Following loading, resin was washed with 20 column volumes of PBS and the IL-2 muteins were eluted using 20 mM sodium acetate, pH 3.5. For bivalent IL-2 muteins, anion exchange (Capto Q, GE Healthcare) pass through following Protein A chromatography was typically sufficient for high homogeneity protein. For monovalent IL-2 muteins, following initial Protein A purification and anion exchange pass-through, cation exchange chromatography (Capto S) was used to separate the desired monovalent heterodimer IL-2 mutein from the contaminants, such as Fc variant homodimer, IL-2 mutein monomer and/or IL-2 mutein homodimer, to achieve greater than 95% monovalent heterodimer. Different variants required different salt and pH conditions for separation. In certain cases, size exclusion chromatography was further used to achieve >95% monovalent heterodimer.

### Example 2. Determination of Binding Affinity of IL-2 Muteins to Human IL2-Ra or IL2-RP by Surface Plasmon Resonance

A surface plasmon resonance (SPR) assay on a Biacore T200 (Cytiva) instrument was used to determine the binding affinities of IL-2 muteins against polyhistidine-tagged IL-2 receptors. Biacore T200 Evaluation Software was used to fit each titration series to a 1:1 binding model or steady state affinity. The association rate constant (kₒₙ, M⁻¹ s⁻¹) and dissociation rate constant (k_{off}, s⁻¹) were determined for each set of titrations and used to calculate the dissociation constant, K_{D}=k_{off}/kₒₙ, of each IL-2 mutein against each receptor.

To measure the affinity of the IL-2 Fc muteins and the WT-IL-2 Fc fusion control for IL-2 receptors, the IL-2 Fc fusion was captured on an anti-human IgG Fc antibody surface followed by the IL-2 receptor as analyte. Exemplary IL-2 muteins 86BCH, 43BGO, 44BGO, 47BJO, 48BJO, 49BJO, 65BJO, 44BJP, 45BJP, 46BJP, and 47BJP are monovalent, whereas 54BGO is a bivalent version of 43BGO, 48BMP is a bivalent version of 49BJO, and 21BMT is a bivalent version of 65BJO. 86BCH contains a T to A substitution corresponding to position 3 and a C to S substitution corresponding to position 125 of wild type human IL-2 (SEQ ID NO:39) and was used as a control. The other exemplary IL-2 muteins all contain the same T to A and C to S substitutions, corresponding to positions 3 and 125 of wild type human IL-2, respectively. Table 1 summarizes the amino acid substitutions introduced into each IL-2 mutein. The positions of such substitutions are according to wild type human IL-2 amino acid sequence (SEQ ID NO:39). It is noted that such substitutions, when described in the context of SEQ ID NO: 1 or 2, will differ since each of SEQ ID NO: 1 and 2 do not contain the first amino acid that is found in the wild type human IL-2 amino acid sequence (SEQ ID NO:39).

TABLE 1
Exemplary IL-2 muteins and corresponding
amino acid substitutions

| | Substitutions compared to wild type human IL-2 |
|---|---|
| IL-2 mutein | (SEQ ID NO: 39) |
| 86BCH or 99BHY | T3A C125S |
| 43BGO | T3A D20N V69A Q74P C125S |
| 44BGO | T3A D20N P34R E68S C125S |
| 54BGO (bivalent) | T3A D20N V69A Q74P C125S |
| 47BJO | T3A D20N P34R V69A Q74P C125S |
| 48BJO | T3A D20N P34R E68S V69A Q74P C125S |
| 49BJO | T3A D20N E68S V69A Q74P C125S |
| 65BJO | T3A D20N E68S Q74P C125S |
| 44BJP | T3A D20N E68S N71R C125S |
| 45BJP | T3A D20N P34R E68S N71R C125S |
| 46BJP | T3A D20N E68S N71R Q74P C125S |
| 47BJP | T3A D20N P34R E68S V69A N71R Q74P C125S |
| 48BMP (bivalent) | T3A D20N E68S V69A Q74P C125S |
| | (same mutations as 49BJO) |
| 21BMT (bivalent) | T3A D20N E68S Q74P C125S |
| | (same mutations as 65BJO) |

As shown in Table 2, the binding affinity of control 86BCH (average of four tests) for human IL-2Rα was around 30 nM, whereas the affinities of the other exemplary IL-2 muteins for human IL-2Rα ranged from 1.1 nM to 44 nM. On the other hand, the affinity of control 86BCH (average of four tests) for human IL-2Rβ was around 1500 nM, whereas none of the other exemplary IL-2 muteins exhibited detectable binding to human IL-2Rβ when assayed at 3 μM.

TABLE 2
Binding Affinities of IL-2 Muteins to Human IL-2-Rα or IL-2-Rβ

| | Binding to IL-2Rα | Binding to IL-2Rβ |
|---|---|---|
| IL-2 Mutein | KD(M) | KD(M) |
| 86BCH | 3.0E−08 ± 1.0E−08 | 1.5E−06 ± 2.3E−07 |
| 43BGO | 1.1E−09 | NB¹ |
| 44BGO | 1.6E−08 | NB |
| 54BGO (bivalent) | 1.9E−09 | NB |
| 47BJO | 1.8E−09 | NB |
| 48BJO | 4.0E−09 | NB |
| 49BJO | 3.9E−09 | NB |
| 65BJO | 4.1E−08 | NB |
| 44BJP | 4.4E−08 | NB |
| 45BJP | 4.2E−08 | NB |
| 46BJP | 2.8E−08 | NB |
| 47BJP | 1.8E−09 | NB |
| 48BMP | 9.8E−09 | NB |
| 21BMT | 9.7E−08 | NB |

¹No binding.

### Example 3. Human Peripheral Blood Mononuclear Cells (PBMC) pSTAT5 Assay

IL-2 muteins were prepared in a serial dilution, and 50 μL was added to a U bottom plate in duplicates. 50 μL PBMCs (˜500 kc/w) were added and the samples were placed at 37° C., 500 CO₂ for 30 minutes (“in”). Cold PBS was added, the samples were centrifuged, and supernatants were removed. IC fixation buffer (Invitrogen Cat 00-8222-49) was added, mixed, and the samples were incubated at room temperature for 20 m. The samples were washed 2 times with FBS staining buffer (BD Cat 554656), and then stained with anti-CD3 (Invitrogen Cat 47-0038-42) and anti-CD56 (Biolegend Cat 318334) antibodies for 30 m, covered on ice. The samples were washed 2 times with staining buffer and permeabilized using prechilled Perm Buffer III (BD Cat 558050) for 30 m, covered on ice. Then the samples were washed 2 times with staining buffer, and stained with anti-CD4 (Invitrogen Cat 46-0047-42), anti-CD8 (BD Cat 555634), anti-CD25 (BD Cat 335807), anti-CD127 (Invitrogen Cat 12-1278-42), anti-pSTAT5 Y694 (BD Cat 612599), and anti-FoxP3 (Invitrogen Cat 48-4777-42) for 1 hour, covered on ice. The samples were washed 2 times with staining buffer and resuspended in 130 μL. Then the samples were analyzed on the BD Symphony flow cytometer.

FCS files were imported into FlowJo or FCSExpress. Lymphocytes were gated using forward scatter versus side scatter. Single cells were gated using forward scatter area versus forward scatter height and sequentially by forward scatter height versus forward scatter width. From there, CD3 negative cells expressing CD56 were gated for NK cells (pSTAT5 expression evaluated from these cells). CD3 positive cells were subgated into CD4 positive or CD8 positive cells (pSTAT5 expression evaluated from these CD8 positive cells). CD4 positive cells were broken down further using CD25 and FoxP3 expression, and cells that were CD4 positive but double negative for CD25 and FoxP3 were identified as Tconv cells (pSTAT5 expression evaluated from these cells). The CD4 positive cells that were double positive for CD25 and FoxP3 were identified as regulatory T cells (pSTAT5 expression evaluated from these cells). Untreated samples were used to guide gating for the pSTAT5 positive cells.

PBMCs were stimulated with serial dilutions of various exemplary IL-2 muteins containing a Fc variant. 86BCH or 99BHY was used as a control. The only difference between 99BHY and 86BCH is that the Fc variant in 99BHY lacks its C-terminus lysine residue. pSTAT5 responses of the other exemplary IL-2 muteins were normalized to 86BCH or 99BHY using the percent positive pSTAT5 at the highest concentration as 100% and no treatment as 0%. Dose response curves were generated using the log(agonist) vs. response—Variable slope (four parameters) fit, and EC50s were calculated.

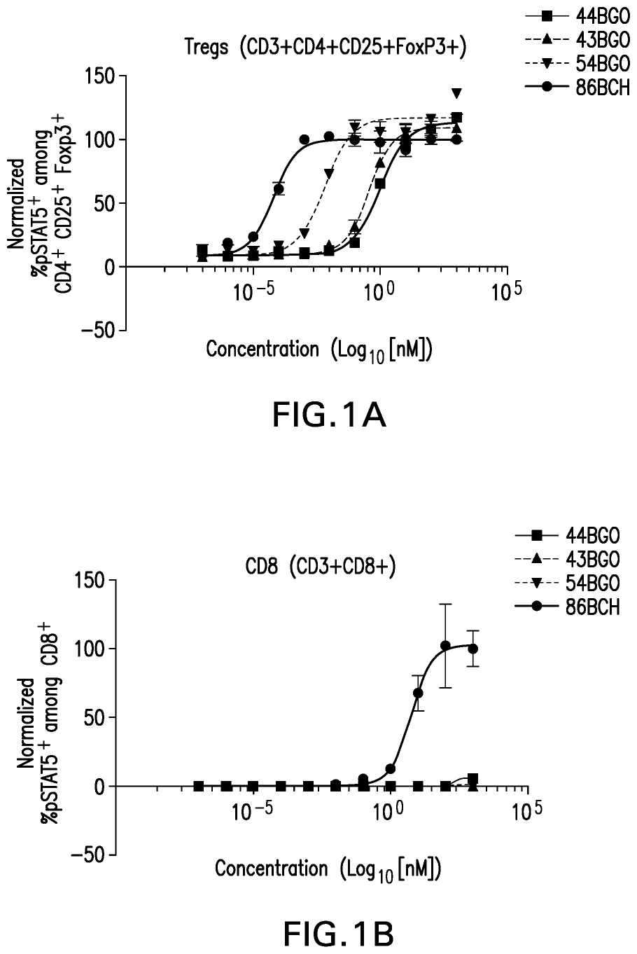

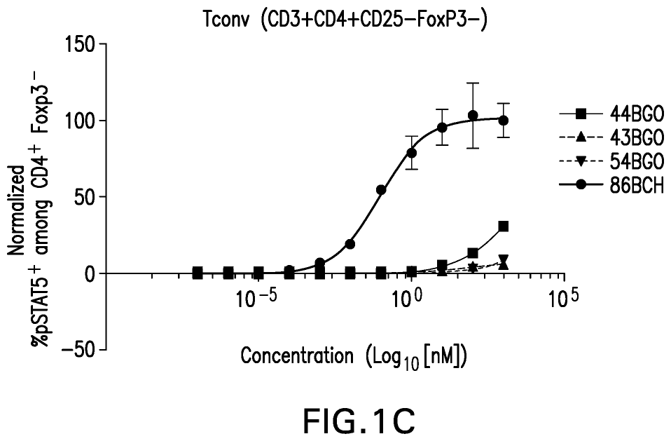

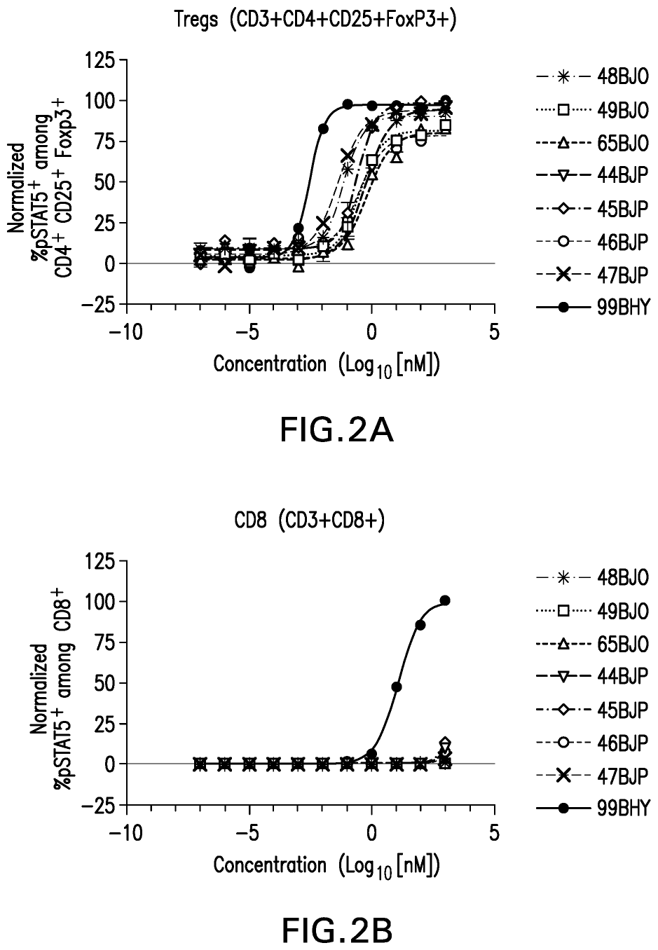

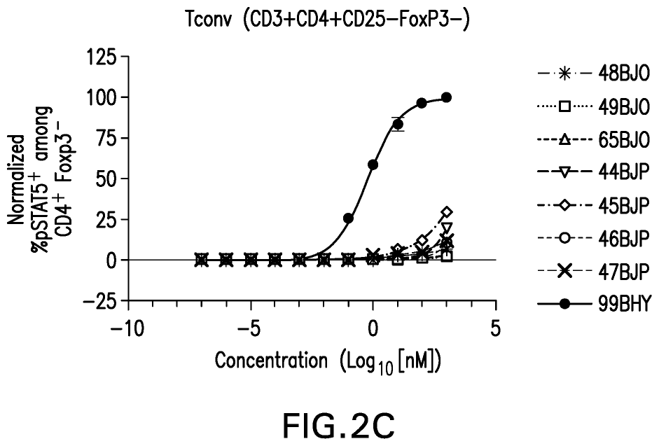

The ability of such IL-2 muteins to activate pSTAT5 was assessed in a mixed population of PBMCs from multiple donors, with gating on CD4⁺CD25⁺Foxp3⁺ regulatory (Treg) cells and effector T cell populations, including CD8⁺ T cells and CD4⁺CD25⁻Foxp3⁻ T cells. Treg cells exhibited a lower pSTAT5 EC50 response to the control as compared to CD8⁺ T cells and CD4⁺CD25⁻Foxp3⁻ T cells, consistent with a higher affinity for the trimeric IL-2 receptor complex (IL-2R α/β/γ) expressed on Treg cells than the affinity for the dimeric IL-2 receptor complex (IL-2R β/γ) expressed on CD8⁺ T cells and CD4⁺CD25⁻Foxp3⁻ T cells (FIGS. 1A-1C for 86BCH and FIGS. 2A-2C for 99BHY). In contrast, stimulation of PBMCs with the other exemplary IL-2 muteins induced STAT5 phosphorylation selectively in Tregs, but not in CD8⁺ and CD4⁺CD25⁻Foxp3⁻ T cells at concentrations up to 1 μM (FIGS. 1A-1C and FIGS. 2A-2C). In line with the preferential activity of these muteins on the CD4⁺CD25⁺Foxp3⁺ Treg cell compartment, induction of STAT5 phosphorylation at doses higher than 0.1 μM was minimal in CD8⁺ T cells and CD4⁺CD25⁻Foxp3⁻ T cells as compared to the controls (FIGS. 1B-1C and FIGS. 2B-2C). These responses were consistent across multiple donors (n=7). These results demonstrate that various exemplary IL-2 muteins selectively activated primary human Treg cells but not CD8⁺ T effector cells or CD4⁺CD25⁻Foxp3⁻ T cells.

The EC50 values of the exemplary IL-2 muteins for activating primary human Treg cells are summarized in Table 3A below.

TABLE 3A
EC50 values of exemplary IL-2 muteins
in activating primary human Treg cells

| IL-2 mutein | EC50 (nM) | Standard deviation (nM) |
|---|---|---|
| 43BGO | 0.227 | 0.227 |
| 44BGO | 1.232 | 0.893 |
| 54BGO (bivalent) | 0.039 | 0.063 |
| 48BJO | 0.216 | 0.229 |
| 49BJO | 0.441 | 0.285 |
| 65BJO | 0.928 | 1.011 |
| 44BJP | 0.334 | 0.271 |
| 45BJP | 0.465 | 0.475 |
| 46BJP | 0.259 | 0.148 |
| 47BJP | 0.153 | 0.178 |
| 99BHY | 0.003 | 0.002 |

The same experiment as described above was run for additional exemplary IL-2 muteins, again using 99BHY as a control, as set forth in Table 3B below.

TABLE 3B
EC50 values of additional exemplary IL-2 muteins
in activating primary human Treg cells

| IL-2 mutein | EC50 (nM) | Standard deviation (nM) |
|---|---|---|
| 99BHY | 0.00258 | 0.00145 |
| DNB558 (monovalent) | 4.5304 | 3.93733 |
| 21BMT (bivalent) | 0.03147 | 0.0215 |
| DNB557 (monovalent) | 0.97162 | 1.08037 |
| 48BMP (bivalent) | 0.01784 | 0.00819 |

DNB558 is monovalent and has the same IL-2 mutations as 65BJO (also monovalent) and 21BMT (bivalent). DNB558 and 65BJO differ in that 65BJO has additional mutations in the Fc region. DNB557 is monovalent and has the same IL-2 mutations as 49BJO (monovalent) and 48BMP (bivalent). DNB557 differs from 49BJO in that 49BJO has additional mutations in the Fc region.

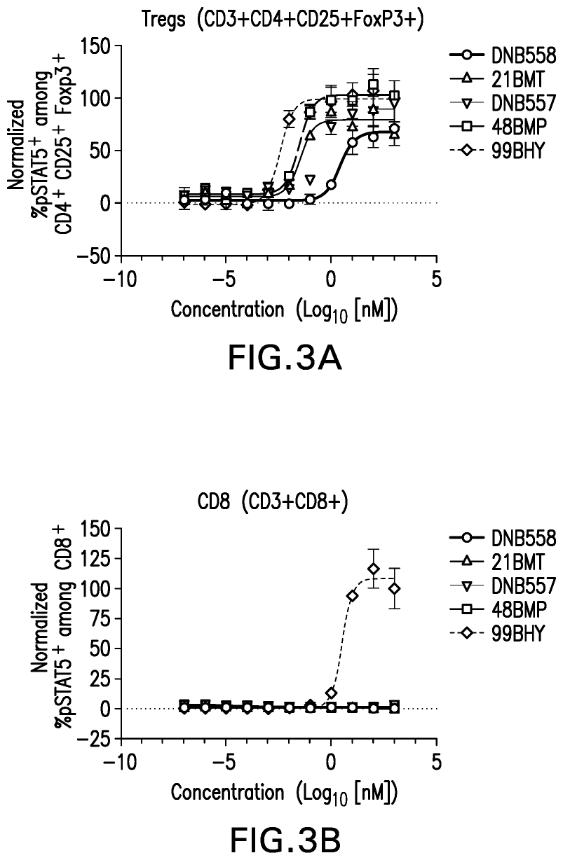

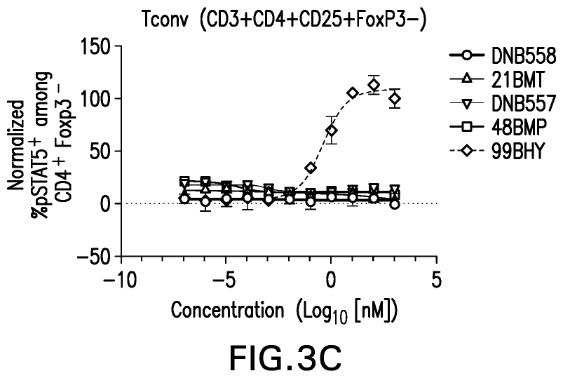

Similarly, Treg cells exhibited a lower pSTAT5 EC50 response to the control as compared to CD8+ T cells and CD4⁺CD25⁻Foxp3⁻ T cells, consistent with a higher affinity for the trimeric IL-2 receptor complex (IL-2R α/β/γ) expressed on Treg cells than the affinity for the dimeric IL-2 receptor complex (IL-2R β/γ) expressed on CD8⁺ T cells and CD4⁺CD25⁻Foxp3⁻ T cells (FIG. 3A for 99BHY). In contrast, stimulation of PBMCs with the additional exemplary IL-2 muteins evaluated induced STAT5 phosphorylation selectively in Tregs, but not in CD8⁺ and CD4⁺CD25⁻Foxp3⁻ T cells at concentrations up to 1 μM (FIGS. 3A-3C). In line with the preferential activity of these muteins on the CD4⁺CD25⁺Foxp3⁺ Treg cell compartment, induction of STAT5 phosphorylation at doses higher than 0.1 μM was minimal in CD8⁺ T cells and CD4⁺CD25⁻Foxp3⁻ T cells as compared to the controls (FIGS. 3B-3C). These responses were consistent across multiple donors (n=7). These results are consistent with the prior results and demonstrate that various exemplary IL-2 muteins selectively activated primary human Treg cells but not CD8⁺ T effector cells or CD4⁺CD25⁻Foxp3⁻ T cells.

### Example 4. Rhesus Monkey Whole Blood pSTAT5 Assay

IL-2 muteins were prepared in a serial dilution, and 25 μL was added to a deep well plate in duplicates, sealed and placed at 37° C., 5% CO₂ for 30 m. 100 μL room temperature Rhesus monkey whole blood was added and mixed. The samples were incubated at 37° C., 5% CO₂ for 20 m. The cells were stained with anti-CD3 (BD Cat 557917), anti-CD127 (Invitrogen Cat 12-1278-42), anti-CD25 (Invitrogen Cat 25-0257-42), and anti-NKG2A (Miltenyi Cat 130-113-565) antibodies and incubated at room temperature for 20 m with gentle shaking, covered. Then the cells were lysed and fixed using 1× Lyse/Fix buffer (BD Cat 558049), mixed well, and incubated at room temperature for 10 m, covered. The cells were washed 2 times with FBS staining buffer (BD Cat 554656) and transferred to a 96 well plate. Prechilled Perm Buffer III (BD Cat 558050) was added to the samples, mixed, and incubated on ice for 30 m, covered. The samples were washed 2 times with staining buffer. Intracellular antibodies anti-FoxP3 (Invitrogen Cat 48-4777-42), anti-pSTAT5 Y694 (BD Cat 612599), anti-CD4 (BD Cat 552838) and anti-CD8 (BD Cat 563795) were added to the samples, incubated for 1 hour at room temperature, covered. The samples were washed 2 times with staining buffer and resuspended in 130 μL. The samples were analyzed on the BD Symphony flow cytometer.

FCS files were imported into FlowJo. Lymphocytes were gated using forward scatter versus side scatter. Single cells were gated using forward scatter area versus forward scatter height. From there, CD3 negative cells expressing NKG2A were gated for NK cells (pSTAT5 expression evaluated from these cells). CD3 positive cells were subgated into CD4 positive or CD8 positive cells (pSTAT5 expression evaluated from these CD8 positive cells). CD4 positive cells were broken down further using CD25 and FoxP3 expression, and cells that were CD4 positive but double negative for CD25 and FoxP3 were identified as Tconv cells (pSTAT5 expression evaluated from these cells). The CD4 positive cells were further subgated into CD25 and CD127. The cells that were CD25 positive and CD127 negative were assessed for FoxP3 expression, and these were identified as regulatory T cells (pSTAT5 expression evaluated from these cells). FMOs for CD25, FoxP3, and pSTAT5 were used to guide gating.

The whole blood was stimulated with serial dilutions of various exemplary IL-2 muteins containing a Fc variant. 99BHY was used as a control. pSTAT5 responses of the other exemplary IL-2 muteins were normalized to 99BHY using the percent positive pSTAT5 at the highest concentration as 100% and no treatment as 0%. Dose response curves were generated using the log(agonist) vs. response—Variable slope (four parameters) fit, and EC50s were calculated.

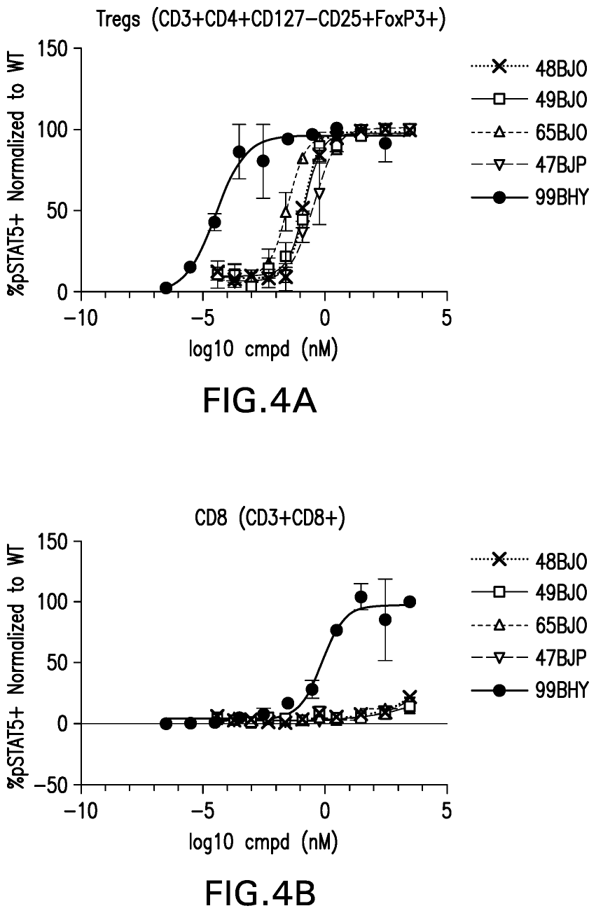

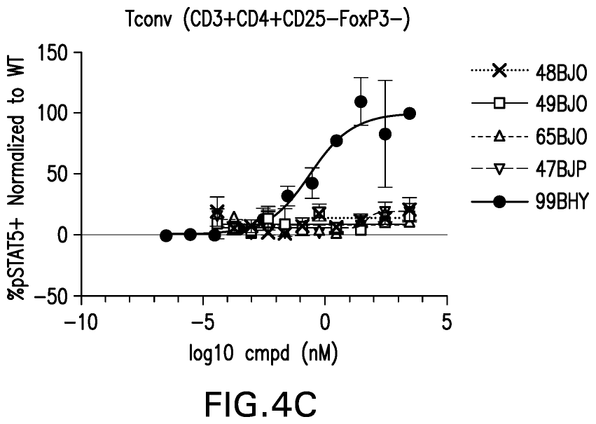

The ability of such IL-2 muteins to activate pSTAT5 was assessed in a whole blood matrix from multiple rhesus donors, with gating on CD4⁺CD127⁻CD25⁺Foxp3⁺ regulatory (Treg) cells and effector T cell populations, including CD8⁺ T cells and CD4⁺CD25⁻Foxp3⁻ T cells. Treg cells exhibited a lower pSTAT5 EC50 response to the control as compared to CD8⁺ T cells and CD4⁺CD25⁻Foxp3⁻ T cells, consistent with a higher affinity for the trimeric IL-2 receptor complex (IL-2R α/β/γ) expressed on Treg cells than the affinity for the dimeric IL-2 receptor complex (IL-2R β/γ) expressed on CD8⁺ T cells and CD4⁺CD25⁻Foxp3⁻ T cells FIGS. 3A-3C for 99BHY). In contrast, stimulation of whole blood with the other exemplary IL-2 muteins induced STAT5 phosphorylation selectively in Tregs, but not in CD8⁺ and CD4⁺CD25⁻Foxp3⁻ T cells at concentrations up to 3 μM (FIGS. 4A-4C). In line with the preferential activity of these muteins on the CD4⁺CD25⁺Foxp3⁺ Treg cell compartment, induction of STAT5 phosphorylation at doses higher than 1 μM was minimal in CD8⁺ T cells and CD4⁺CD25⁻Foxp3⁻ T cells as compared to the control (FIGS. 4B-4C). These responses were consistent across multiple donors (n=2). These results demonstrate that various exemplary IL-2 muteins selectively activated primary rhesus Treg cells but not CD8⁺ T effector cells or CD4⁺CD25⁻Foxp3⁻ T cells.

The EC50 values of the exemplary IL-2 muteins for activating primary rhesus Treg cells are summarized in Table 4A below.

TABLE 4A
EC50 values of exemplary IL-2 muteins
in activating primary rhesus Treg cells

| IL-2 mutein | EC50 (nM) | Standard deviation (nM) |
|---|---|---|
| 48BJO | 0.165 | 0.037 |
| 49BJO | 0.199 | 0.059 |
| 65BJO | 0.023 | 0.007 |
| 47BJP | 0.224 | 0.087 |

The same experiment as described above was run for additional exemplary IL-2 muteins, again using 99BHY as a control, as set forth in Table 4B below. DNB558 is monovalent and has the same IL-2 mutations as 65BJO (also monovalent) and 21BMT (bivalent). DNB557 is monovalent and has the same IL-2 mutations as 49BJO (monovalent) and 48BMP (bivalent).

TABLE 4B
EC50 values of exemplary IL-2 muteins
in activating primary rhesus Treg cells

| IL-2 mutein | EC50 (nM) | Standard deviation (nM) |
|---|---|---|
| 99BHY | 0.000751 | 0.000166 |
| DNB558 (monovalent) | 0.069823 | 0.010048 |
| 21BMT (bivalent) | 0.008134 | 0.007411 |
| DNB557 (monovalent) | 0.6894 | 0.152767 |
| 48BMP (bivalent) | 0.023936 | 0.020688 |

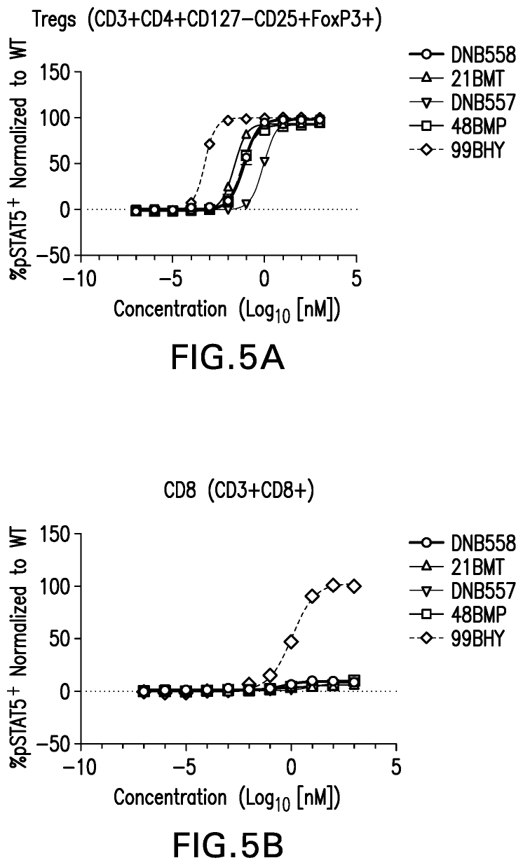

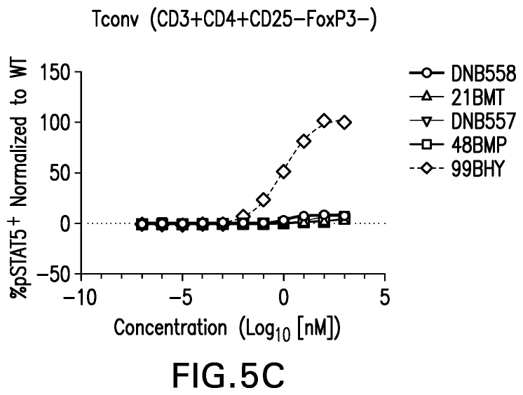

Similarly, Treg cells exhibited a lower pSTAT5 EC50 response to the control as compared to CD8⁺ T cells and CD4⁺CD25⁻Foxp3⁻ T cells, consistent with a higher affinity for the trimeric IL-2 receptor complex (IL-2R α/β/γ) expressed on Treg cells than the affinity for the dimeric IL-2 receptor complex (IL-2R β/γ) expressed on CD8⁺ T cells and CD4⁺CD25⁻Foxp3⁻ T cells (FIG. 5A for 99BHY). In contrast, stimulation of PBMCs with the additional exemplary IL-2 muteins evaluated induced STAT5 phosphorylation selectively in Tregs, but not in CD8⁺ and CD4⁺CD25⁻Foxp3⁻ T cells at concentrations up to 1 μM (FIGS. 5A-5C). In line with the preferential activity of these muteins on the CD4⁺CD25⁺Foxp3⁺ Treg cell compartment, induction of STAT5 phosphorylation at doses higher than 0.1 μM was minimal in CD8⁺ T cells and CD4⁺CD25⁻Foxp3⁻ T cells as compared to the controls (FIGS. 5B-5C). These responses were consistent across multiple donors (n=7). These results are consistent with the prior results and demonstrate that various exemplary IL-2 muteins selectively activated primary human Treg cells but not CD8⁺ T effector cells or CD4⁺CD25⁻Foxp3⁻ T cells.

### Example 5. Human Treg Expansion and Activation in Xeno-GvHD Model

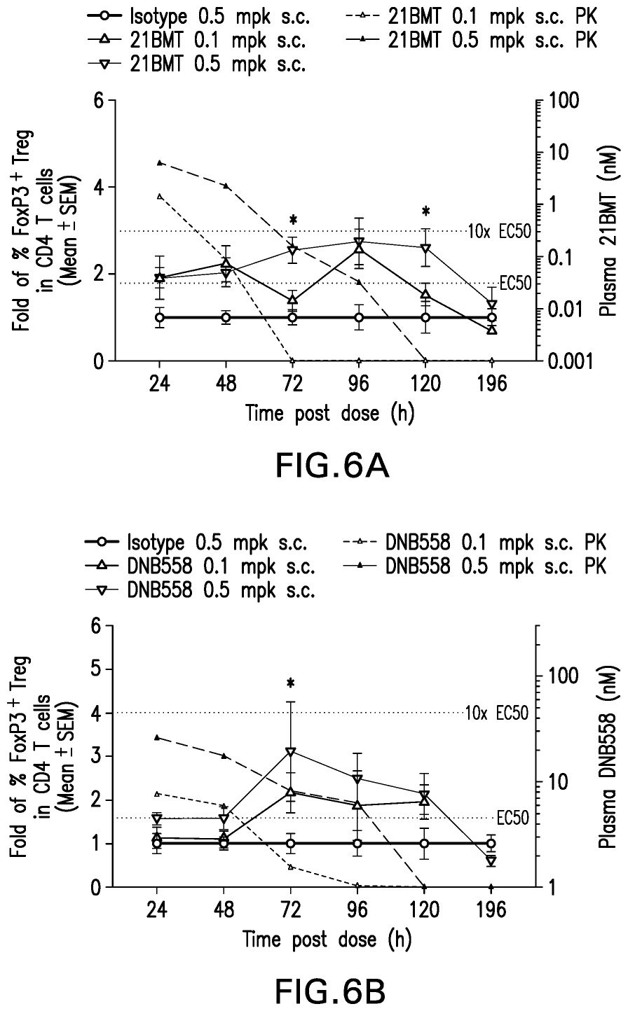

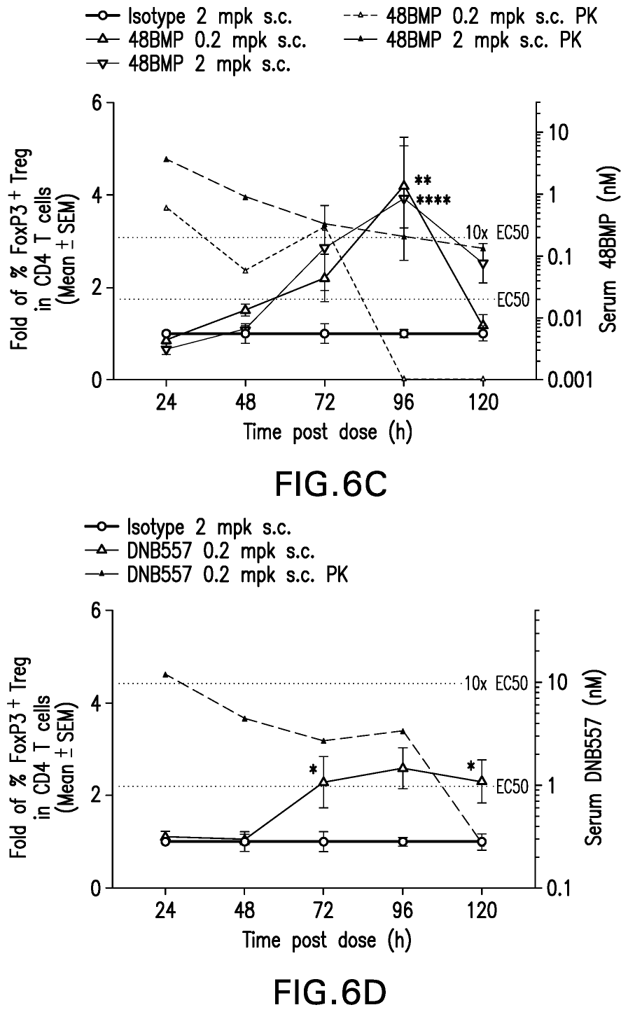

Muteins were evaluated for their ability to expand and activate human Tregs in a xeno-GvHD (Graft vs. Host Disease) model using huPBMC-NSG mice. NSG mice were purchased from the Jackson Laboratory and human PMBC were engrafted into the mice. Fourteen to seventeen days post human PBMC (peripheral blood mononuclear cell) engraftment, the mice were treated with one dose of IL-2 muteins. The exemplary IL-2 muteins evaluated in this example are set forth below in Table 5. Splenocytes from the mice were profiled daily for five days or eight days in some studies by FACS (fluorescence activated cell sorting) analysis. Human Tregs in the splenocytes were identified as human CD45⁺CD56⁻CD3⁺CD4⁺CD127^{low}Foxp3⁺ T cells. All muteins evaluated increased human CD4⁺ FoxP3⁺ Treg expansion (FIGS. 6A-6D).

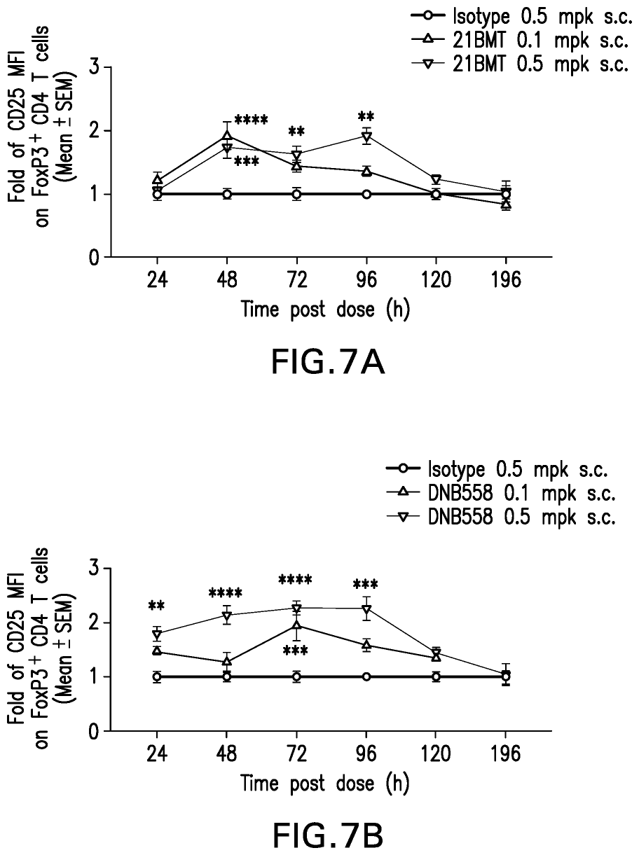

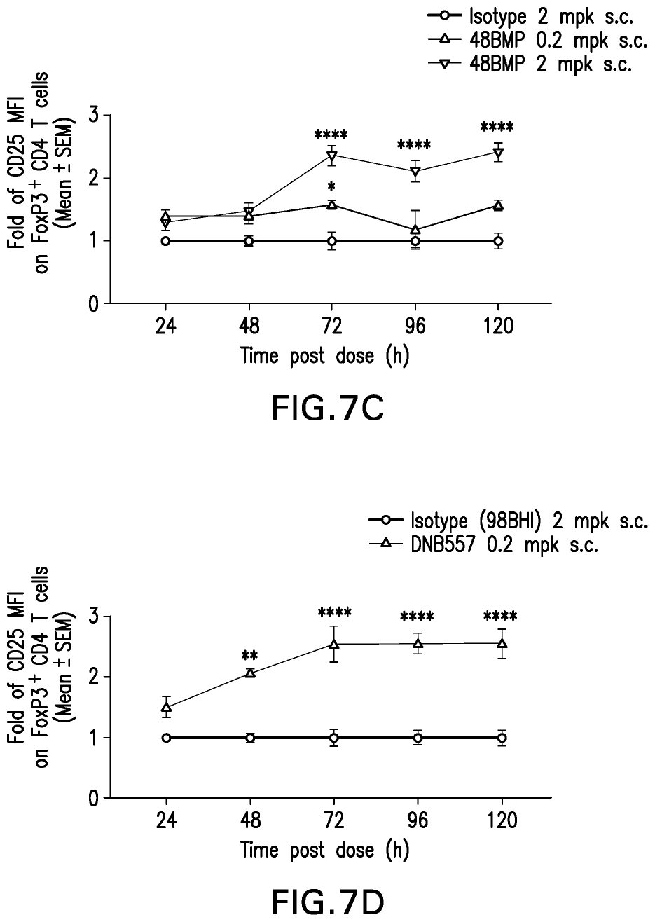

The surface expression of CD25 on human Tregs was quantified by FACS analysis. All IL-2 muteins evaluated increased CD25 expression on the surface of Tregs (FIGS. 7A-7D).

TABLE 5
Exemplary IL-2 muteins evaluated

| | Substitutions compared to wild type |
|---|---|
| IL-2 mutein | human IL-2 (SEQ ID NO: 39) |
| 48BMP (bivalent) | T3A D20N E68S V69A Q74P C125S |
| | (same mutations as 49BJO) |
| 21BMT (bivalent) | T3A D20N E68S Q74P C125S |
| | (same mutations as 65BJO) |
| DNB557 (monovalent) | T3A D20N E68S V69A Q74P C125S |
| DNB558 (monovalent) | T3A D20N E68S Q74P C125S |

### Example 6. PF/PK Profiles for Muteins in Monovalent and Bivalent Format in Rhesus Monkeys

The PK/PD profile of certain exemplary IL-2 muteins was evaluated where the muteins were in bivalent and monovalent formats following subcutaneous delivery. The study design is as set forth below in Table 6. Biologic naïve rhesus male monkeys were utilized in the study. 3 animals per group were used. Animals were dosed on day 0 and 14 via subcutaneous route of administration and the dosing solution volume was 1 mL/kg. The duration of the study was 28 days.

TABLE 6

| Group | IL-2 Mutein | Dose Level (ug/kg) |
|---|---|---|
| 1 | 48BMP (bivalent) | 900 |
| 2 | 21BMT (bivalent) | 100 |
| 3 | DNB557 | 900 |
| 4 | DNB558 | 100 |

Pharmacokinetic (PK) serum (7 aliquots of 100 uL each) was obtained as follows: Day 0 1^{st} dose: predose, 15 min, 2 hr, 6 hr; Day 1, Day 2, Day 3, Day 7, Day 9; Day 14 2^{nd} dose: predose, 15 min, 2 hr, 6 hr; Days 15, 16, 17, 21, 23, 28. Whole blood (2 aliquots) was collected for pSTAT5 assay and immunophenotypying (Treg, Tconv, CD8, NK cells) for pharmacodynamic (PD) analysis was obtained as follows:
Panel A: Day −5, Day 0 1st dose: predose; Day 1, Day 3, Day 4, Day 7, Day 9; Day 14 2nd dose: predose; Days 15, 17, 18, 21, 23, 28
Panel B: Day −5, Day 0 1st dose: predose, 2 hr; Day 1, Day 2, Day 3, Day 7, Day 9; Day 14 2nd dose: predose, 2 hr; Days 15, 16, 17, 21, 23, 28

Serum chemistry and CRP (c-reactive protein) was evaluated on the following: Day −5, Day 0 1^{st} dose: predose, 2 hr; Day 1, Day 7; Day 14 2^{nd} dose: predose, 2 hr; Days 15, 16, 17, 21, 23, 28. Hematology was evaluated on the following: Day −5, Day 0 1^{st} dose: predose; Day 1, Day 3, Day 4, Day 7, Day 9; Day 14 2^{nd} dose: predose; Days 15, 17, 18, 21, 23, 28.

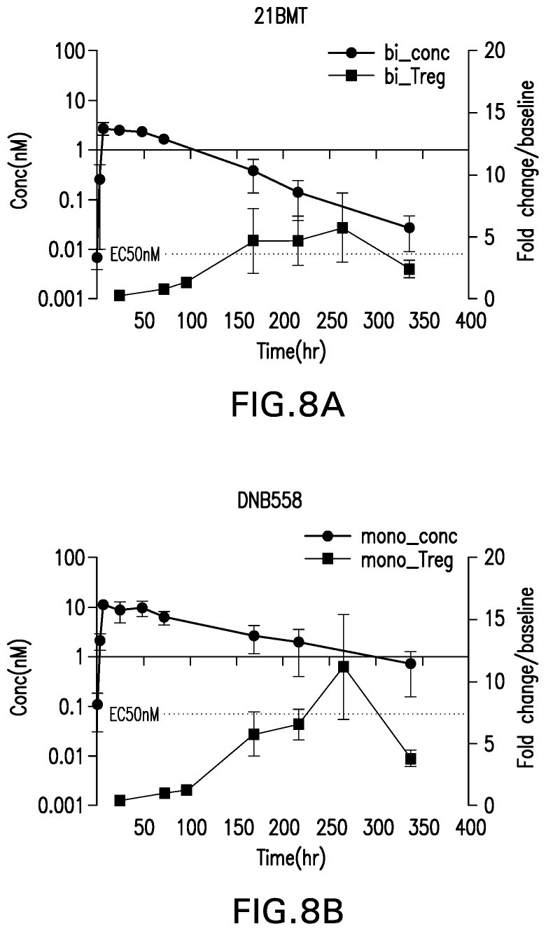

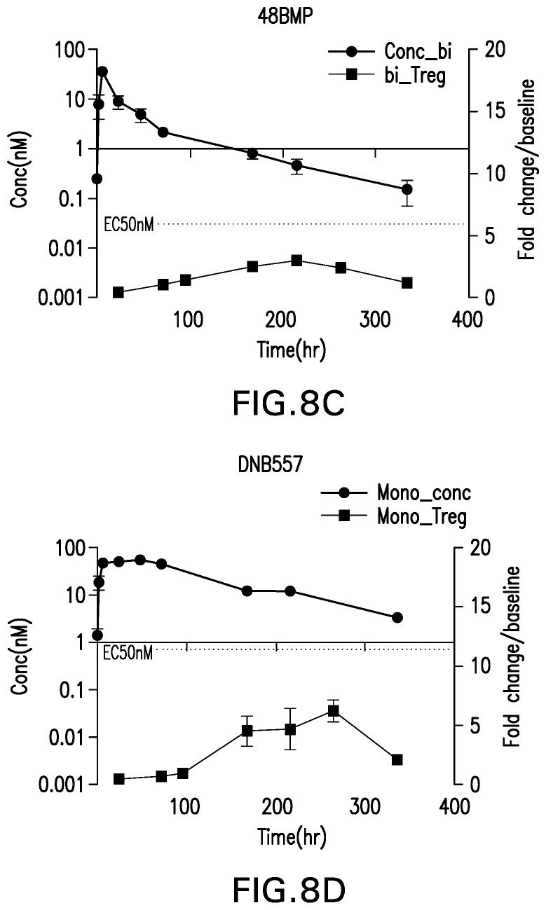

The PK/PD results after dose 1 are set forth below for both the monovalent and bivalent muteins (see Tables 7A and 7B, respectively) and in FIGS. 8A-8D.

TABLE 7A
Monovalent NCA data after dose 1

| | | Half | | | | CL-F- | |
|---|---|---|---|---|---|---|---|
| | | life | Tmax | Cmax | AUClast | pred | MRTlast |
| Mutein | Animal | (hr) | (hr) | (nmol/L)) | (hr*nmol/L) | (L/hr/kg) | (hr) |
| DNB558 | 1 | 66.2 | 6 | 11.1 | 996 | 0.00146 | 86.6 |
| | 2 | 101 | 48 | 13.5 | 1920 | 0.000706 | 107 |
| | 3 | 75 | 6 | 11.2 | 1010 | 0.00143 | 81.3 |
| | N | 3 | 3 | 3 | 3 | 3 | 3 |
| | Mean | 80.7 | 20 | 11.9 | 1,310 | 0.0012 | 91.6 |
| | SD | 18 | 24.2 | 1.34 | 531 | 0.000427 | 13.5 |
| DNB557 | 4 | 70 | 24 | 57.1 | 7440 | 0.00175 | 85.2 |
| | 5 | 70.2 | 24 | 54.9 | 7730 | 0.00168 | 96.1 |
| | 6 | 71.3 | 48 | 64.6 | 7460 | 0.00174 | 85.3 |
| | N | 3 | 3 | 3 | 3 | 3 | 3 |
| | Mean | 70.5 | 32 | 58.9 | 7,540 | 0.00172 | 88.9 |
| | SD | 0.71 | 13.9 | 5.1 | 163 | 3.81E−05 | 6.28 |

TABLE 7B
Bivalent NCA data after dose 1

| | | Half | | | | CL-F- | |
|---|---|---|---|---|---|---|---|
| | | life | Tmax | Cmax | AUClast | pred | MRTlast |
| Mutein | Animal | (hr) | (hr) | (nmol/L)) | (hr*nmol/L) | (L/hr/kg) | (hr) |
| 21BMT | 7 | 38.7 | 6 | 2.41 | 302 | 0.00401 | 79 |
| | 8 | 41.8 | 24 | 2.38 | 231 | 0.00525 | 56.2 |
| | 9 | 48.9 | 6 | 3.68 | 329 | 0.00367 | 66.3 |
| | N | 3 | 3 | 3 | 3 | 3 | 3 |
| | Mean | 43.1 | 12 | 2.83 | 287 | 0.00431 | 67.2 |
| | SD | 5.24 | 10.4 | 0.743 | 50.5 | 0.000831 | 11.4 |
| 48BMP | 10 | 53.1 | 6 | 33.7 | 820 | 0.0133 | 38.9 |
| | 11 | 70.8 | 6 | 32.5 | 855 | 0.0126 | 50.6 |
| | 12 | 74.9 | 6 | 39.1 | 1110 | 0.00969 | 44.1 |
| | N | 3 | 3 | 3 | 3 | 3 | 3 |
| | Mean | 66.3 | 6 | 35.1 | 929 | 0.0118 | 44.5 |
| | SD | 11.6 | 0 | 3.52 | 158 | 0.0019 | 5.87 |

In addition, Table 8 sets forth various PK/PD results for the exemplary IL-2 muteins evaluated along with rhesus STAT5 and human STAT5 results. The results for the Rhesus and Human STAT5 assay in Table 8 below were calculated using the geometric mean.

TABLE 8

| | | Rhesus | Human | Treg | | | |
|---|---|---|---|---|---|---|---|
| | Dose | STAT5 | STAT5 | fold | MRT | AUC_Treg/ | AUG_Treg/ |
| Mutein | (nmol/kg) | nM | nM | change | (hr) | AUC_exp | Dose |
| DNB558 | 1.5 | 0.066 | 5.02 | 11.2 | 91.6 | 1.2 | 1044 |
| monovalent | | | | | | | |
| DNB557 | 14.5 | 0.723 | 1.12 | 6.2 | 88.9 | 0.1 | 99 |
| monovalent | | | | | | | |
| 21BMT | 1.2 | 0.008 | 0.03 | 5.8 | 67.2 | 3.6 | 867 |
| bivalent | | | | | | | |
| 48BMP | 11 | 0.028 | 0.02 | 2.8 | 44.5 | 0.6 | 48 |
| bivalent | | | | | | | |

All references cited herein are incorporated by reference to the same extent as if each individual publication, database entry (e.g. GenBank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. GenBank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

Table 9 below summaries all sequences disclosed in the specification.

TABLE 9
Sequences disclosed in the specification

| SEQ | | |
|---|---|---|
| ID | Sequence | |
| NO | Description | Sequence (protein) |
| 1 | Aldesleukin | PTSSSTKKTQLQLEHLLLDLQMILNGINNYKNPKLTRMLTFKFYMPKKATEL |
| | | KHLQCLEEELKPLEEVLNLAQSKNFHLRPRDLISNINVIVLELKGSETTFMCE |
| | | YADETATIVEFLNRWITFSQSIISTLT |
| 2 | Aldsleukin_T3A | PASSSTKKTQLQLEHLLLDLQMILNGINNYKNPKLTRMLTFKFYMPKKATEL |
| | | KHLQCLEEELKPLEEVLNLAQSKNFHLRPRDLISNINVIVLELKGSETTFMCE |
| | | YADETATIVEFLNRWITFSQSIISTLT |
| 3 | 43BGO-IL-2 | PASSSTKKTQLQLEHLLLNLQMILNGINNYKNPKLTRMLTFKFYMPKKATEL |
| | | KHLQCLEEELKPLEEALNLAPSKNFHLRPRDLISNINVIVLELKGSETTFMCE |
| | | YADETATIVEFLNRWITFSQSIISTLT |
| 4 | 44BGO-IL-2 | PASSSTKKTQLQLEHLLLNLQMILNGINNYKNRKLTRMLTFKFYMPKKATEL |
| | | KHLQCLEEELKPLESVLNLAQSKNFHLRPRDLISNINVIVLELKGSETTFMCE |
| | | YADETATIVEFLNRWITFSQSIISTLT |
| 5 | 47BJO-IL-2 | PASSSTKKTQLQLEHLLLNLQMILNGINNYKNRKLTRMLTFKFYMPKKATEL |
| | | KHLQCLEEELKPLEEALNLAPSKNFHLRPRDLISNINVIVLELKGSETTFMCE |
| | | YADETATIVEFLNRWITFSQSIISTLT |
| 6 | 48BJO-IL-2 | PASSSTKKTQLQLEHLLLNLQMILNGINNYKNRKLTRMLTFKFYMPKKATEL |
| | | KHLQCLEEELKPLESALNLAPSKNFHLRPRDLISNINVIVLELKGSETTFMCE |
| | | YADETATIVEFLNRWITFSQSIISTLT |
| 7 | 49BJO-IL-2 | PASSSTKKTQLQLEHLLLNLQMILNGINNYKNPKLTRMLTFKFYMPKKATEL |
| | | KHLQCLEEELKPLESALNLAPSKNFHLRPRDLISNINVIVLELKGSETTFMCE |
| | | YADETATIVEFLNRWITFSQSIISTLT |
| 8 | 65BJO-IL-2 | PASSSTKKTQLQLEHLLLNLQMILNGINNYKNPKLTRMLTFKFYMPKKATEL |
| | | KHLQCLEEELKPLESVLNLAPSKNFHLRPRDLISNINVIVLELKGSETTFMCE |
| | | YADETATIVEFLNRWITFSQSIISTLT |
| 9 | 44BJP-IL-2 | PASSSTKKTQLQLEHLLLNLQMILNGINNYKNPKLTRMLTFKFYMPKKATEL |
| | | KHLQCLEEELKPLESVLRLAQSKNFHLRPRDLISNINVIVLELKGSETTFMCE |
| | | YADETATIVEFLNRWITFSQSIISTLT |
| 10 | 45BJP-IL-2 | PASSSTKKTQLQLEHLLLNLQMILNGINNYKNRKLTRMLTFKFYMPKKATEL |
| | | KHLQCLEEELKPLESVLRLAQSKNFHLRPRDLISNINVIVLELKGSETTFMCE |
| | | YADETATIVEFLNRWITFSQSIISTLT |
| 11 | 46BJP-IL-2 | PASSSTKKTQLQLEHLLLNLQMILNGINNYKNPKLTRMLTFKFYMPKKATEL |
| | | KHLQCLEEELKPLESVLRLAPSKNFHLRPRDLISNINVIVLELKGSETTFMCE |
| | | YADETATIVEFLNRWITFSQSIISTLT |
| 12 | 47BJP-IL-2 | PASSSTKKTQLQLEHLLLNLQMILNGINNYKNRKLTRMLTFKFYMPKKATEL |
| | | KHLQCLEEELKPLESALRLAPSKNFHLRPRDLISNINVIVLELKGSETTFMCE |
| | | YADETATIVEFLNRWITFSQSIISTLT |
| 13 | Fc-1 | DKTHTCPPCPAPEAAGGPSVFLFPPKPKDTLMISRTPEVTCVVVSVSHEDP |
| | | EVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 14 | Fc-2 | DKTHTCPPCPAPEAAGGPSVFLFPPKPKDTLMISRTPEVTCVVVSVSHEDP |
| | | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPG |
| 15 | Fc-3 | DKTHTCPPCPAPEAAGGPSVFLFPPKPKDTLMISRTPEVTCVVVSVSHEDP |
| | | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPG |
| 16 | Linker-1 | GGGGSGGGGS |
| 17 | Fc-4 | DKTHTCPPCPAPEAAGGPSVFLFPPKPKDTLMISRTPEVTCVVVSVSHEDP |
| | | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPGK |
| 18 | Fc-5 | DKTHTCPPCPAPEAAGGPSVFLFPPKPKDTLMISRTPEVTCVVVSVSHEDP |
| | | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGK |
| 19 | 43BGO with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGGSGGGGSPASSSTKKTQLQLE |
| | | HLLLNLQMILNGINNYKNPKLTRMLTFKFYMPKKATELKHLQCLEEELKPLEE |
| | | ALNLAPSKNFHLRPRDLISNINVIVLELKGSETTFMCEYADETATIVEFLNRWI |
| | | TFSQSIISTLT |
| 20 | 44BGO with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGGSGGGGSPASSSTKKTQLQLE |
| | | HLLLNLQMILNGINNYKNRKLTRMLTFKFYMPKKATELKHLQCLEEELKPLE |
| | | SVLNLAQSKNFHLRPRDLISNINVIVLELKGSETTFMCEYADETATIVEFLNR |
| | | WITFSQSIISTLT |
| 21 | 47BJO with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGGSGGGGSPASSSTKKTQLQLE |
| | | HLLLNLQMILNGINNYKNRKLTRMLTFKFYMPKKATELKHLQCLEEELKPLE |
| | | EALNLAPSKNFHLRPRDLISNINVIVLELKGSETTFMCEYADETATIVEFLNR |
| | | WITFSQSIISTLT |
| 22 | 48BJO with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGGSGGGGSPASSSTKKTQLQLE |
| | | HLLLNLQMILNGINNYKNRKLTRMLTFKFYMPKKATELKHLQCLEEELKPLE |
| | | SALNLAPSKNFHLRPRDLISNINVIVLELKGSETTFMCEYADETATIVEFLNR |
| | | WITFSQSIISTLT |
| 23 | 49BJO with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGGSGGGGSPASSSTKKTQLQLE |
| | | HLLLNLQMILNGINNYKNPKLTRMLTFKFYMPKKATELKHLQCLEEELKPLES |
| | | ALNLAPSKNFHLRPRDLISNINVIVLELKGSETTFMCEYADETATIVEFLNRWI |
| | | TFSQSIISTLT |
| 24 | 65BJO with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGGSGGGGSPASSSTKKTQLQLE |
| | | HLLLNLQMILNGINNYKNPKLTRMLTFKFYMPKKATELKHLQCLEEELKPLES |
| | | VLNLAPSKNFHLRPRDLISNINVIVLELKGSETTFMCEYADETATIVEFLNRWI |
| | | TFSQSIISTLT |
| 25 | 44BJP with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGGSGGGGSPASSSTKKTQLQLE |
| | | HLLLNLQMILNGINNYKNPKLTRMLTFKFYMPKKATELKHLQCLEEELKPLES |
| | | VLRLAQSKNFHLRPRDLISNINVIVLELKGSETTFMCEYADETATIVEFLNRWI |
| | | TFSQSIISTLT |
| 26 | 45BJP with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGGSGGGGSPASSSTKKTQLQLE |
| | | HLLLNLQMILNGINNYKNRKLTRMLTFKFYMPKKATELKHLQCLEEELKPLE |
| | | SVLRLAQSKNFHLRPRDLISNINVIVLELKGSETTFMCEYADETATIVEFLNR |
| | | WITFSQSIISTLT |
| 27 | 46BJP with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGGSGGGGSPASSSTKKTQLQLE |
| | | HLLLNLQMILNGINNYKNPKLTRMLTFKFYMPKKATELKHLQCLEEELKPLES |
| | | VLRLAPSKNFHLRPRDLISNINVIVLELKGSETTFMCEYADETATIVEFLNRWI |
| | | TFSQSIISTLT |
| 28 | 47BJP with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGGSGGGGSPASSSTKKTQLQLE |
| | | HLLLNLQMILNGINNYKNRKLTRMLTFKFYMPKKATELKHLQCLEEELKPLE |
| | | SALRLAPSKNFHLRPRDLISNINVIVLELKGSETTFMCEYADETATIVEFLNR |
| | | WITFSQSIISTLT |
| 29 | 43BGO with Fc | DKTHTCPPCPAPEAAGGPSVFLFPPKPKDTLMISRTPEVTCVVVSVSHEDP |
| | hole | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPGGGGGSGGGGSPASSSTKKTQLQLEHL |
| | | LLNLQMILNGINNYKNPKLTRMLTFKFYMPKKATELKHLQCLEEELKPLEEAL |
| | | NLAPSKNFHLRPRDLISNINVIVLELKGSETTFMCEYADETATIVEFLNRWITF |
| | | SQSIISTLT |
| 30 | 44BGO with Fc | DKTHTCPPCPAPEAAGGPSVFLFPPKPKDTLMISRTPEVTCVVVSVSHEDP |
| | hole | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPGGGGGSGGGGSPASSSTKKTQLQLEHL |
| | | LLNLQMILNGINNYKNRKLTRMLTFKFYMPKKATELKHLQCLEEELKPLESVL |
| | | NLAQSKNFHLRPRDLISNINVIVLELKGSETTFMCEYADETATIVEFLNRWITF |
| | | SQSIISTLT |
| 31 | 47BJO with Fc | DKTHTCPPCPAPEAAGGPSVFLFPPKPKDTLMISRTPEVTCVVVSVSHEDP |
| | hole | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPGGGGGSGGGGSPASSSTKKTQLQLEHL |
| | | LLNLQMILNGINNYKNRKLTRMLTFKFYMPKKATELKHLQCLEEELKPLEEAL |
| | | NLAPSKNFHLRPRDLISNINVIVLELKGSETTFMCEYADETATIVEFLNRWITF |
| | | SQSIISTLT |
| 32 | 48BJO with Fc | DKTHTCPPCPAPEAAGGPSVFLFPPKPKDTLMISRTPEVTCVVVSVSHEDP |
| | hole | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPGGGGGSGGGGSPASSSTKKTQLQLEHL |
| | | LLNLQMILNGINNYKNRKLTRMLTFKFYMPKKATELKHLQCLEEELKPLESAL |
| | | NLAPSKNFHLRPRDLISNINVIVLELKGSETTFMCEYADETATIVEFLNRWITF |
| | | SQSIISTLT |
| 33 | 49BJO with Fc | DKTHTCPPCPAPEAAGGPSVFLFPPKPKDTLMISRTPEVTCVVVSVSHEDP |
| | hole | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPGGGGGSGGGGSPASSSTKKTQLQLEHL |
| | | LLNLQMILNGINNYKNPKLTRMLTFKFYMPKKATELKHLQCLEEELKPLESAL |
| | | NLAPSKNFHLRPRDLISNINVIVLELKGSETTFMCEYADETATIVEFLNRWITF |
| | | SQSIISTLT |
| 34 | 65BJO with Fc | DKTHTCPPCPAPEAAGGPSVFLFPPKPKDTLMISRTPEVTCVVVSVSHEDP |
| | hole | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPGGGGGSGGGGSPASSSTKKTQLQLEHL |
| | | LLNLQMILNGINNYKNPKLTRMLTFKFYMPKKATELKHLQCLEEELKPLESVL |
| | | NLAPSKNFHLRPRDLISNINVIVLELKGSETTFMCEYADETATIVEFLNRWITF |
| | | SQSIISTLT |
| 35 | 44BJP with Fc | DKTHTCPPCPAPEAAGGPSVFLFPPKPKDTLMISRTPEVTCVVVSVSHEDP |
| | hole | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPGGGGGSGGGGSPASSSTKKTQLQLEHL |
| | | LLNLQMILNGINNYKNPKLTRMLTFKFYMPKKATELKHLQCLEEELKPLESVL |
| | | RLAQSKNFHLRPRDLISNINVIVLELKGSETTFMCEYADETATIVEFLNRWITF |
| | | SQSIISTLT |
| 36 | 45BJP with Fc | DKTHTCPPCPAPEAAGGPSVFLFPPKPKDTLMISRTPEVTCVVVSVSHEDP |
| | hole | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPGGGGGSGGGGSPASSSTKKTQLQLEHL |
| | | LLNLQMILNGINNYKNRKLTRMLTFKFYMPKKATELKHLQCLEEELKPLESVL |
| | | RLAQSKNFHLRPRDLISNINVIVLELKGSETTFMCEYADETATIVEFLNRWITF |
| | | SQSIISTLT |
| 37 | 46BJP with Fc | DKTHTCPPCPAPEAAGGPSVFLFPPKPKDTLMISRTPEVTCVVVSVSHEDP |
| | hole | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPGGGGGSGGGGSPASSSTKKTQLQLEHL |
| | | LLNLQMILNGINNYKNPKLTRMLTFKFYMPKKATELKHLQCLEEELKPLESVL |
| | | RLAPSKNFHLRPRDLISNINVIVLELKGSETTFMCEYADETATIVEFLNRWITF |
| | | SQSIISTLT |
| 38 | 47BJP with Fc | DKTHTCPPCPAPEAAGGPSVFLFPPKPKDTLMISRTPEVTCVVVSVSHEDP |
| | hole | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNHYTQKSLSLSPGGGGGSGGGGSPASSSTKKTQLQLEHL |
| | | LLNLQMILNGINNYKNRKLTRMLTFKFYMPKKATELKHLQCLEEELKPLESAL |
| | | RLAPSKNFHLRPRDLISNINVIVLELKGSETTFMCEYADETATIVEFLNRWITF |
| | | SQSIISTLT |
| 39 | Human wild type | APTSSSTKKTQLQLEHLLLDLQMILNGINNYKNPKLTRMLTFKFYMPKKATE |
| | IL-2 | LKHLQCLEEELKPLEEVLNLAQSKNFHLRPRDLISNINVIVLELKGSETTFMC |
| | | EYADETATIVEFLNRWITFCQSIISTLT |
| 40 | Human wild type | PTSSSTKKTQLQLEHLLLDLQMILNGINNYKNPKLTRMLTFKFYMPKKATEL |
| | IL-2 minus A1 | KHLQCLEEELKPLEEVLNLAQSKNFHLRPRDLISNINVIVLELKGSETTFMCE |
| | | YADETATIVEFLNRWITFCQSIISTLT |
| 41 | Linker-2 | GGGSGGGS |
| 42 | Linker-3 | GGGGGSGGGGGS |
| 43 | 99BHY with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGGSGGGGSPASSSTKKTQLQLE |
| | | HLLLDLQMILNGINNYKNPKLTRMLTFKFYMPKKATELKHLQCLEEELKPLEE |
| | | VLNLAQSKNFHLRPRDLISNINVIVLELKGSETTFMCEYADETATIVEFLNRWI |
| | | TFSQSIISTLT |
| 44 | 86BCH with Fc | DKTHTCPPCPAPEAAGGPSVFLFPPKPKDTLMISRTPEVTCVVVSVSHEDP |
| | knob | EVKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGN |
| | | VFSCSVMHEALHNHYTQKSLSLSPGKGGGGSGGGGSPASSSTKKTQLQL |
| | | EHLLLDLQMILNGINNYKNPKLTRMLTFKFYMPKKATELKHLQCLEEELKPLE |
| | | EVLNLAQSKNFHLRPRDLISNINVIVLELKGSETTFMCEYADETATIVEFLNR |
| | | WITFSQSIISTLT |
| 45 | 21BMT | DKTHTCPPCPAPEAAGGPSVFLFPPKPKDTLMISRTPEVTCVVVSVSHEDP |
| | Same mutations as | EVKFNWYVDGVEVHNAKTKPREEQYNSTYRVVSVLTVLHQDWLNGKEYK |
| | 65BJO with linker | CKVSNKALPAPIEKTISKAKGQPREPQVYTLPPSRDELTKNQVSLTCLVKGF |
| | and FC-1 | YPSDIAVEWESNGQPENNYKTTPPVLDSDGSFFLYSKLTVDKSRWQQGNV |
| | | FSCSVMHEALHNHYTQKSLSLSPGGGGGSGGGGSPASSSTKKTQLQLEHL |
| | | LLNLQMILNGINNYKNPKLTRMLTFKFYMPKKATELKHLQCLEEELKPLESVL |
| | | NLAPSKNFHLRPRDLISNINVIVLELKGSETTFMCEYADETATIVEFLNRWITF |
| | | SQSIISTLT |
| 46 | 48BMP | DKTHTCPPCPAPEAAGGPSVFLFPPKPKDTLMISRTPEVTCVVVSVSHEDP |
| | Same mutations as | EVKFNWYVDGVEVHNAKTKPREEQYNSTYRVVSVLTVLHQDWLNGKEYK |
| | 49BJO with linker | CKVSNKALPAPIEKTISKAKGQPREPQVYTLPPSRDELTKNQVSLTCLVKGF |
| | and Fc-1 | YPSDIAVEWESNGQPENNYKTTPPVLDSDGSFFLYSKLTVDKSRWQQGNV |
| | | FSCSVMHEALHNHYTQKSLSLSPGGGGGSGGGGSPASSSTKKTQLQLEHL |
| | | LLNLQMILNGINNYKNPKLTRMLTFKFYMPKKATELKHLQCLEEELKPLESAL |
| | | NLAPSKNFHLRPRDLISNINVIVLELKGSETTFMCEYADETATIVEFLNRWITF |
| | | SQSIISTLT |
| 47 | FC-1 with linker | DKTHTCPPCPAPEAAGGPSVFLFPPKPKDTLMISRTPEVTCVVVSVSHEDP |
| | and 43BGO IL-2 | EVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPGGGGGSGGGGSPASSSTKKTQLQLEHL |
| | | LLNLQMILNGINNYKNPKLTRMLTFKFYMPKKATELKHLQCLEEELKPLEEAL |
| | | NLAPSKNFHLRPRDLISNINVIVLELKGSETTFMCEYADETATIVEFLNRWITF |
| | | SQSIISTLT |
| 48 | FC-1 with linker | DKTHTCPPCPAPEAAGGPSVFLFPPKPKDTLMISRTPEVTCVVVSVSHEDP |
| | and 44BGO IL-2 | EVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPGGGGGSGGGGSPASSSTKKTQLQLEHL |
| | | LLNLQMILNGINNYKNRKLTRMLTFKFYMPKKATELKHLQCLEEELKPLESVL |
| | | NLAQSKNFHLRPRDLISNINVIVLELKGSETTFMCEYADETATIVEFLNRWITF |
| | | SQSIISTLT |
| 49 | FC-1 with linker | DKTHTCPPCPAPEAAGGPSVFLFPPKPKDTLMISRTPEVTCVVVSVSHEDP |
| | and 47BJO IL-2 | EVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPGGGGGSGGGGSPASSSTKKTQLQLEHL |
| | | LLNLQMILNGINNYKNRKLTRMLTFKFYMPKKATELKHLQCLEEELKPLEEAL |
| | | NLAPSKNFHLRPRDLISNINVIVLELKGSETTFMCEYADETATIVEFLNRWITF |
| | | SQSIISTLT |
| 50 | FC-1 with linker | DKTHTCPPCPAPEAAGGPSVFLFPPKPKDTLMISRTPEVTCVVVSVSHEDP |
| | and 48BJO | EVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPGGGGGSGGGGSPASSSTKKTQLQLEHL |
| | | LLNLQMILNGINNYKNRKLTRMLTFKFYMPKKATELKHLQCLEEELKPLESAL |
| | | NLAPSKNFHLRPRDLISNINVIVLELKGSETTFMCEYADETATIVEFLNRWITF |
| | | SQSIISTLT |
| 51 | FC-1 with linker | DKTHTCPPCPAPEAAGGPSVFLFPPKPKDTLMISRTPEVTCVVVSVSHEDP |
| | and 44BJP IL-2 | EVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPGGGGGSGGGGSPASSSTKKTQLQLEHL |
| | | LLNLQMILNGINNYKNPKLTRMLTFKFYMPKKATELKHLQCLEEELKPLESVL |
| | | RLAQSKNFHLRPRDLISNINVIVLELKGSETTFMCEYADETATIVEFLNRWITF |
| | | SQSIISTLT |
| 52 | FC-1 with linker | DKTHTCPPCPAPEAAGGPSVFLFPPKPKDTLMISRTPEVTCVVVSVSHEDP |
| | and 45BJP IL-2 | EVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPGGGGGSGGGGSPASSSTKKTQLQLEHL |
| | | LLNLQMILNGINNYKNRKLTRMLTFKFYMPKKATELKHLQCLEEELKPLESVL |
| | | RLAQSKNFHLRPRDLISNINVIVLELKGSETTFMCEYADETATIVEFLNRWITF |
| | | SQSIISTLT |
| 53 | FC-1 with linker | DKTHTCPPCPAPEAAGGPSVFLFPPKPKDTLMISRTPEVTCVVVSVSHEDP |
| | and 46BJP IL-2 | EVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPGGGGGSGGGGSPASSSTKKTQLQLEHL |
| | | LLNLQMILNGINNYKNPKLTRMLTFKFYMPKKATELKHLQCLEEELKPLESVL |
| | | RLAPSKNFHLRPRDLISNINVIVLELKGSETTFMCEYADETATIVEFLNRWITF |
| | | SQSIISTLT |
| 54 | FC-1 with linker | DKTHTCPPCPAPEAAGGPSVFLFPPKPKDTLMISRTPEVTCVVVSVSHEDP |
| | and 47BJP IL-2 | EVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPGGGGGSGGGGSPASSSTKKTQLQLEHL |
| | | LLNLQMILNGINNYKNRKLTRMLTFKFYMPKKATELKHLQCLEEELKPLESAL |
| | | RLAPSKNFHLRPRDLISNINVIVLELKGSETTFMCEYADETATIVEFLNRWITF |
| | | SQSIISTLT |
| 55 | Fc-6 | DKTHTCPPCPAPEAAGGPSVFLFPPKPKDTLMISRTPEVTCVVVSVSHEDP |
| | | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNRFTQKSLSLSPGK |
| 56 | Fc-7 | DKTHTCPPCPAPEAAGGPSVFLFPPKPKDTLMISRTPEVTCVVVSVSHEDP |
| | | EVKFNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNV |
| | | FSCSVMHEALHNRFTQKSLSLSPG |

1. An IL-2 mutein comprising a first polypeptide comprising the amino acid sequence as set forth in SEQ ID NO:1 or 2 in which the D at position 19 of SEQ ID NO:1 or 2 is substituted with N and the P at position 33 of SEQ ID NO:1 or 2 is substituted with R, wherein the polypeptide optionally comprises one or more additional amino acid substitutions relative to SEQ ID NO:1 or 2.

2. An IL-2 mutein comprising a first polypeptide comprising the amino acid sequence as set forth in SEQ ID NO:1 or 2 in which the D at position 19 of SEQ ID NO:1 or 2 is substituted with N and the E at position 67 of SEQ ID NO:1 or 2 is substituted with S wherein the polypeptide optionally comprises one or more additional amino acid substitutions relative to SEQ ID NO:1 or 2.

3. The IL-2 mutein of claim 2, wherein the first polypeptide further comprises an E to S substitution at position 67 of SEQ ID NO:1 or 2.

4. The IL-2 mutein of claim 2, wherein the first polypeptide further comprises a V to A substitution at position 68, an N to R substitution at position 70, or a Q to P substitution at position 73 of SEQ ID NO:1 or 2.

5. The IL-2 mutein of claim 2, wherein the first polypeptide further comprises any two of the three following substitutions: a V to A substitution at position 68, an N to R substitution at position 70, or a Q to P substitution at position 73 of SEQ ID NO:1 or 2.

6. The IL-2 mutein of claim 2, wherein the first polypeptide further comprises a V to A substitution at position 68, an N to R substitution at position 70, and a Q to P substitution at position 73 of SEQ ID NO:1 or 2.

7. An IL-2 mutein comprising a first polypeptide comprising an amino acid sequence as set forth in SEQ ID NO:3, 4, 5, 6, 7, 8, 9, 10, 11, or 12.

8. The IL-2 mutein of claim 2, wherein the first polypeptide further comprises an amino acid sequence as set forth in SEQ ID NO:13, 14, or 15.

9. The IL-2 mutein of claim 8, wherein the first polypeptide further comprises a linker as set forth in SEQ ID NO:16.

10. The IL-2 mutein of claim 8, further comprising a second polypeptide, wherein
(1) the first polypeptide comprises an amino acid sequence as set forth in SEQ ID NO:14 and the second polypeptide comprises an amino acid sequence as set forth in SEQ ID NO:17; or
(2) the first polypeptide comprises an amino acid sequence as set forth in SEQ ID NO:15 and the second polypeptide comprises an amino acid sequence as set forth in SEQ ID NO:18.

11-16. (canceled)

17. An IL-2 mutein comprising a first polypeptide and a second polypeptide, wherein each of the first and second polypeptide comprise the same amino acid sequence which comprises an amino acid sequence as set forth in SEQ ID NO: 45, 46, 47, 48, 49, 50, 51, 52, 53, or 54.

18. The IL-2 mutein of claim 17, wherein the first polypeptide comprises an amino acid sequence of SEQ ID NO: 45 and the second polypeptide comprises an amino acid sequence as set forth in SEQ ID NO: 45.

19. The IL-mutein of claim 17, wherein the first polypeptide comprises an amino acid sequence of SEQ ID NO: 46 and the second polypeptide comprises an amino acid sequence of SEQ ID NO: 46.

20. A pharmaceutical composition comprising the IL-2 mutein of claim 2, and a pharmaceutically acceptable carrier.

21-22. (canceled)

23. A method of treating an IL-2-mediated disease in a subject, comprising administering to the subject a therapeutically effective amount of the IL-2 mutein of claim 1, wherein the IL-2-mediated disease is rheumatoid arthritis, Crohn's disease, psoriasis, psoriatic arthritis, multiple sclerosis, systemic lupus erythematosus (SLE), cutaneous lupus erythematosus (CLE), lupus nephritis, ankylosing spondylitis, type I diabetes, Sjogren's syndrome, ulcerative colitis, neuromyelitis optica, celiac disease, scleroderma, temporal arteritis, atopic dermatitis, alopecia areata, graft versus host disease (GVHD), autoimmune hepatitis, primary sclerosing cholangitis, or inflammatory myopathy.

24-25. (canceled)

26. An isolated nucleic acid comprising a sequence of nucleotides that encodes the first polypeptide of the IL-2 mutein of claim 2.

27. An expression vector comprising the isolated nucleic acid of claim 26.

28. A host cell comprising the expression vector of claim 27.

29. A method of producing an IL-2 mutein, comprising:
(a) culturing the host cell of claim 28;
under conditions wherein the IL-2 mutein is expressed.

30-44. (canceled)

45. A pharmaceutical composition comprising the IL-2 mutein of claim 18, and a pharmaceutically acceptable carrier.

46. An isolated nucleic acid comprising a sequence of nucleotides that encodes the second polypeptide of the IL-2 mutein of claim 17.

47. An isolated nucleic acid comprising a sequence of nucleotides that encodes the first and the second polypeptides of the IL-2 mutein of any one of claim 17.

48. An expression vector comprising the isolated nucleic acid of claim 47.


SEQUENCE LISTING (56 sequences)

SEQ ID NO: 1 - protein, 132 aa, Artificial Sequence
  Aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 132 aa, Artificial Sequence
  Aldsleukin_T3A
PASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 132 aa, Artificial Sequence
  43BGO-IL-2
PASSSTKKTQLQLEHLLLNLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 4 - protein, 132 aa, Artificial Sequence
  44BGO-IL-2
PASSSTKKTQLQLEHLLLNLQMILNGINNYKNRKLTRMLTFKFYMPKKATELKHLQCLEEELKPLESVLNLAQSKNFHLRPRDLISNINVIVLELKGSETTFMCEYADETATIVEFLNRWITFSQSIISTLT

SEQ ID NO: 5 - protein, 132 aa, Artificial Sequence
  47BJO-IL-2
PASSSTKKTQLQLEHLLLNLQMILNGINNYKNRKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 6 - protein, 132 aa, Artificial Sequence
  48BJO-IL-2
PASSSTKKTQLQLEHLLLNLQMILNGINNYKNRKLTRMLTFKFYMPKKATELKHLQCLEEELKPLESALNLAPSKNFHLRPRDLISNINVIVLELKGSETTFMCEYADETATIVEFLNRWITFSQSIISTLT

SEQ ID NO: 7 - protein, 132 aa, Artificial Sequence
  49BJO-IL-2
PASSSTKKTQLQLEHLLLNLQMILNGINNYKNPKLTRMLTFKFYMPKKATELKHLQCLEEELKPLESALNLAPSKNFHLRPRDLISNINVIVLELKGSETTFMCEYADETATIVEFLNRWITFSQSIISTLT

SEQ ID NO: 8 - protein, 132 aa, Artificial Sequence
  65BJO-IL-2
PASSSTKKTQLQLEHLLLNLQMILNGINNYKNPKLTRMLTFKFYMPKKATELKHLQCLEEELKPLESVLNLAPSKNFHLRPRDLISNINVIVLELKGSETTFMCEYADETATIVEFLNRWITFSQSIISTLT

SEQ ID NO: 9 - protein, 132 aa, Artificial Sequence
  44BJP-IL-2
PASSSTKKTQLQLEHLLLNLQMILNGINNYKNPKLTRMLTFKFYMPKKATELKHLQCLEEELKPLESVLRLAQSKNFHLRPRDLISNINVIVLELKGSETTFMCEYADETATIVEFLNRWITFSQSIISTLT

SEQ ID NO: 10 - protein, 132 aa, Artificial Sequence
  45BJP-IL-2
PASSSTKKTQLQLEHLLLNLQMILNGINNYKNRKLTRMLTFKFYMPKKATELKHLQCLEEELKPLESVLRLAQSKNFHLRPRDLISNINVIVLELKGSETTFMCEYADETATIVEFLNRWITFSQSIISTLT

SEQ ID NO: 11 - protein, 132 aa, Artificial Sequence
  46BJP-IL-2
PASSSTKKTQLQLEHLLLNLQMILNGINNYKNPKLTRMLTFKFYMPKKATELKHLQCLEEELKPLESVLRLAPSKNFHLRPRDLISNINVIVLELKGSETTFMCEYADETATIVEFLNRWITFSQSIISTLT

SEQ ID NO: 12 - protein, 132 aa, Artificial Sequence
  47BJP-IL-2
PASSSTKKTQLQLEHLLLNLQMILNGINNYKNRKLTRMLTFKFYMPKKATELKHLQCLEEELKPLESALRLAPSKNFHLRPRDLISNINVIVLELKGSETTFMCEYADETATIVEFLNRWITFSQSIISTLT

SEQ ID NO: 13 - protein, 226 aa, Artificial Sequence
  Fc-1
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 226 aa, Artificial Sequence
  Fc-2
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 226 aa, Artificial Sequence
  Fc-3
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 16 - protein, 10 aa, Linker-1
GGGGSGGGGS

SEQ ID NO: 17 - protein, 227 aa, Artificial Sequence
  Fc-4
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 227 aa, Artificial Sequence
  Fc-5
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 368 aa, Artificial Sequence
  43BGO-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 20 - protein, 368 aa, Artificial Sequence
  44BGO-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESVLNLAQSKNFHLRPRDLISNINVIVLELKGSETTFMCEYADETATIVEFLNRWITFSQSIISTLT

SEQ ID NO: 21 - protein, 368 aa, Artificial Sequence
  47BJO-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 22 - protein, 368 aa, Artificial Sequence
  48BJO-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESALNLAPSKNFHLRPRDLISNINVIVLELKGSETTFMCEYADETATIVEFLNRWITFSQSIISTLT

SEQ ID NO: 23 - protein, 368 aa, Artificial Sequence
  49BJO-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESALNLAPSKNFHLRPRDLISNINVIVLELKGSETTFMCEYADETATIVEFLNRWITFSQSIISTLT

SEQ ID NO: 24 - protein, 368 aa, Artificial Sequence
  65BJO-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESVLNLAPSKNFHLRPRDLISNINVIVLELKGSETTFMCEYADETATIVEFLNRWITFSQSIISTLT

SEQ ID NO: 25 - protein, 368 aa, Artificial Sequence
  44BJP-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESVLRLAQSKNFHLRPRDLISNINVIVLELKGSETTFMCEYADETATIVEFLNRWITFSQSIISTLT

SEQ ID NO: 26 - protein, 368 aa, Artificial Sequence
  45BJP-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESVLRLAQSKNFHLRPRDLISNINVIVLELKGSETTFMCEYADETATIVEFLNRWITFSQSIISTLT

SEQ ID NO: 27 - protein, 368 aa, Artificial Sequence
  46BJP-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESVLRLAPSKNFHLRPRDLISNINVIVLELKGSETTFMCEYADETATIVEFLNRWITFSQSIISTLT

SEQ ID NO: 28 - protein, 368 aa, Artificial Sequence
  47BJP-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESALRLAPSKNFHLRPRDLISNINVIVLELKGSETTFMCEYADETATIVEFLNRWITFSQSIISTLT

SEQ ID NO: 29 - protein, 368 aa, Artificial Sequence
  43BGO-Fc hole
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 30 - protein, 368 aa, Artificial Sequence
  44BGO-Fc hole
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESVLNLAQSKNFHLRPRDLISNINVIVLELKGSETTFMCEYADETATIVEFLNRWITFSQSIISTLT

SEQ ID NO: 31 - protein, 368 aa, Artificial Sequence
  47BJO-Fc hole
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 32 - protein, 368 aa, Artificial Sequence
  48BJO-Fc hole
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESALNLAPSKNFHLRPRDLISNINVIVLELKGSETTFMCEYADETATIVEFLNRWITFSQSIISTLT

SEQ ID NO: 33 - protein, 368 aa, Artificial Sequence
  49BJO-Fc hole
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESALNLAPSKNFHLRPRDLISNINVIVLELKGSETTFMCEYADETATIVEFLNRWITFSQSIISTLT

SEQ ID NO: 34 - protein, 368 aa, Artificial Sequence
  65BJO-Fc hole
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESVLNLAPSKNFHLRPRDLISNINVIVLELKGSETTFMCEYADETATIVEFLNRWITFSQSIISTLT

SEQ ID NO: 35 - protein, 368 aa, Artificial Sequence
  44BJP-Fc hole
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESVLRLAQSKNFHLRPRDLISNINVIVLELKGSETTFMCEYADETATIVEFLNRWITFSQSIISTLT

SEQ ID NO: 36 - protein, 368 aa, Artificial Sequence
  45BJP-Fc hole
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESVLRLAQSKNFHLRPRDLISNINVIVLELKGSETTFMCEYADETATIVEFLNRWITFSQSIISTLT

SEQ ID NO: 37 - protein, 368 aa, Artificial Sequence
  46BJP-Fc hole
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESVLRLAPSKNFHLRPRDLISNINVIVLELKGSETTFMCEYADETATIVEFLNRWITFSQSIISTLT

SEQ ID NO: 38 - protein, 368 aa, Artificial Sequence
  47BJP-Fc hole
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESALRLAPSKNFHLRPRDLISNINVIVLELKGSETTFMCEYADETATIVEFLNRWITFSQSIISTLT

SEQ ID NO: 39 - protein, 133 aa, homo sapien [sequence omitted]

SEQ ID NO: 40 - protein, 132 aa, Homo sapiens [sequence omitted]

SEQ ID NO: 41 - protein, 8 aa, Artificial Sequence
  Linker-2
GGGSGGGS

SEQ ID NO: 42 - protein, 12 aa, Artificial Sequence
  Linker-3
GGGGGSGGGGGS

SEQ ID NO: 43 - protein, 368 aa, Artificial Sequence
  99BHY-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 44 - protein, 369 aa, Artificial Sequence
  86BCH-Fc knob
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 45 - protein, 368 aa, Artificial Sequence
  21BMT
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESVLNLAPSKNFHLRPRDLISNINVIVLELKGSETTFMCEYADETATIVEFLNRWITFSQSIISTLT

SEQ ID NO: 46 - protein, 368 aa, Artificial Sequence
  48BMP
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESALNLAPSKNFHLRPRDLISNINVIVLELKGSETTFMCEYADETATIVEFLNRWITFSQSIISTLT

SEQ ID NO: 47 - protein, 368 aa, Artificial Sequence
  FC 1, linker, 43BGO
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 48 - protein, 368 aa, Artificial Sequence
  FC 1, linker 44BGO
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESVLNLAQSKNFHLRPRDLISNINVIVLELKGSETTFMCEYADETATIVEFLNRWITFSQSIISTLT

SEQ ID NO: 49 - protein, 368 aa, Artificial Sequence
  FC 1, linker 47BJO
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 50 - protein, 368 aa, Artificial Sequence
  FC 1, linker 49BJO
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESALNLAPSKNFHLRPRDLISNINVIVLELKGSETTFMCEYADETATIVEFLNRWITFSQSIISTLT

SEQ ID NO: 51 - protein, 368 aa, Artificial Sequence
  FC 1, linker, 44BJP
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESVLRLAQSKNFHLRPRDLISNINVIVLELKGSETTFMCEYADETATIVEFLNRWITFSQSIISTLT

SEQ ID NO: 52 - protein, 368 aa, Artificial Sequence
  FC 1, linker, 45BJP
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESVLRLAQSKNFHLRPRDLISNINVIVLELKGSETTFMCEYADETATIVEFLNRWITFSQSIISTLT

SEQ ID NO: 53 - protein, 368 aa, Artificial Sequence
  FC 1m linker 46BJP
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNPKLTRMLTFKFYMPKKATELKHLQCLEEELKPLESVLRLAPSKNFHLRPRDLISNINVIVLELKGSETTFMCEYADETATIVEFLNRWITFSQSIISTLT

SEQ ID NO: 54 - protein, 368 aa, Artificial Sequence
  FC 1, linker, 47BJP
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLNLQMILNGINNYKNRKLTRMLTFKFYMPKKATELKHLQCLEEELKPLESALRLAPSKNFHLRPRDLISNINVIVLELKGSETTFMCEYADETATIVEFLNRWITFSQSIISTLT

SEQ ID NO: 55 - protein, 227 aa, Artificial Sequence
  Fc-6
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 56 - protein, 226 aa, Artificial Sequence
  Fc-7
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG